(19) United States
(12) Patent Application Publication
(10) Pub. No.: US 2017/0191032 A1
(43) Pub. Date: Jul. 6, 2017

(54) IDENTIFICATION AND ISOLATION OF NEURAL STEM CELLS AND NEUROSPHERE INITIATING CELLS

(71) Applicant: The Board of Regents of the University of Texas System, Austin, TX (US)

(72) Inventors: Sean J. MORRISON, Dallas, TX (US); John K. MICH, Racine, WA (US)

(21) Appl. No.: 15/308,671

(22) PCT Filed: Apr. 27, 2015
(86) PCT No.: PCT/US15/27702

§ 371 Date: Nov. 3, 2016

Related U.S. Application Data
(60) Provisional application No. 61/989,281, filed on May 6, 2014.

(51) Int. Cl. C12N 5/0793

(52) U.S. Cl. CPC C12N 5/0619; G01N 2015/1477

(57) ABSTRACT

The disclosure reports on the identification and isolation of adult mouse lateral ventricle subventricular zone (SVZ) neurosphere initiating cells (NICs) by flow cytometry on the basis of Glast^{mid}EGFR^{high}PlexinB2^{high}CD24^{-/low}O4/PSA-NCAM^{-/low}Ter-119/CD45⁻ markers (GEPCOT cells). These cells are highly mitotic and short-lived in vivo based on fate-mapping with Ascl1^{CreERT2} and Dlx1^{CreERT2}. In contrast, pre-GEPCOT cells were quiescent, expressed higher Glast, and lower EGFR and PlexinB2. Pre-GEP-COT cells could not form neurospheres but expressed the stem cell markers Glast-CreER^{T}, GFAP-CreER^{T2}, Sox2^{CreERT2}, and Gli1^{C-reERT2} and were long-lived in vivo. While GEPCOT NICs were ablated by temozolomide, pre-GEPCOT cells survived and repopulated the SVZ. Conditional deletion of the Bmi-1 polycomb protein depleted pre-GEPCOT and GEPCOT cells, though pre-GEPCOT cells were more dependent upon Bmi-1 for p16^{Ink4a} repression. These data distinguish quiescent NSCs from NICs and make it possible to study their properties in vivo.

This application claims benefit of priority to U.S. Provisional Application Ser. No. 61/989,281, filed May 6, 2014, the entire contents of which are hereby incorporated by reference.

### BACKGROUND

This invention was made with government support under grant no. R37 AG024945 awarded by the National Institutes of Aging. The government has certain rights in the invention.

I. Technical Field

The present disclosure relates generally to the fields of neurology and developmental biology. More particularly, it relates to the identification of surface markers for neural stem cells and neurosphere initiating cells, as well as methods for isolating the same.

II. Related Art

Neural stem cells (NSCs) reside in two regions of the adult mammalian forebrain: the subgranular zone in the dentate gyms and the subventricular zone in the lateral wall of the lateral ventricle (SVZ). SVZ NSCs persist throughout adult life (Maslov et al., 2004; Molofsky et al., 2006; Imayoshi et al., 2008), giving rise primarily to neurons in the olfactory bulb as well as some astrocytes in the olfactory bulb (Lois and Alvarez-Buylla, 1994; Lois et al., 1996; Doetsch et al., 1999b; Ahn and Joyner, 2005; Kuo et al., 2006; Lagace et al., 2007; Merkle et al., 2007; Imayoshi et al., 2008; Chen et al., 2009) and oligodendrocytes in the corpus callosum and cortex (Nait-Oumesmar et al., 1999; Menn et al., 2006). In vivo, these NSCs are quiescent (Doetsch et al., 1999a; Pastrana et al., 2009), resistant to anti-mitotic agents (Morshead et al., 1994; Doetsch et al., 1999b; Doetsch et al., 2002; Giachino and Taylor, 2009), long-lived (Ahn and Joyner, 2005; Imayoshi et al., 2008), and capable of regenerating the SVZ after injury (Doetsch et al., 1999b; Doetsch et al., 2002; López-Juárez et al., 2013).

Clonal colony-forming assays have been widely used to study neural stem/progenitor cells that give rise to neurons, astrocytes, and oligodendrocytes in adherent cultures (Davis and Temple, 1994) and non-adherent neurosphere cultures (Reynolds and Weiss, 1992). However, in the adult forebrain it remains uncertain whether these colonies are formed by quiescent neural stem cells (qNSCs) or by mitotically active and shorter-lived multipotent progenitors (Morshead et al., 1994; Doetsch et al., 2002; Reynolds and Rietze, 2005; Pastrana et al., 2009; Pastrana et al., 2011). Moreover, reliance upon retrospective colony-formation assays makes it impossible to directly study qNSCs or NICs as they exist in vivo.

Pioneering work by Alvarez-Buylla and colleagues has demonstrated the existence of a lineage of NSCs and transit amplifying cells in the SVZ that gives rise to neuronal progenitors throughout life (Lois and Alvarez-Buylla, 1994; Doetsch et al., 1997; Doetsch et al., 1999a; Mirzadeh et al., 2008; Ihrie and Alvarez-Buylla, 2011). By electron microscopy and immunofluorescence analysis they identified GFAP-expressing type B cells that appear to be the qNSCs. These cells are resistant to anti-mitotic agents such as AraC and appear capable of repopulating the SVZ after AraC treatment (Doetsch et al., 1999a; Doetsch et al., 1999b; Doetsch et al., 2002). Type B cells are thought to give rise to Ascl1- and Dlx2-expressing type C cells, which are mitotically active and ablated by anti-mitotic agents (Morshead et al., 1994; Doetsch et al., 1999b; Doetsch et al., 2002; Pastrana et al., 2009). The type C cells give rise to Dcx- and PSA-NCAM-expressing type A neuronal progenitors. These studies have provided a critical framework for understanding the SVZ neurogenic lineage, though the inability to purify live cells from each stage of this hierarchy has hampered efforts to assess their properties.

The inability to prospectively identify and isolate uncultured stem cells from the central nervous system (CNS) has contributed to uncertainty regarding the relationship between qNSCs and NICs. SVZ NICs have been enriched by flow cytometry based on CD15 expression (Capela and Temple, 2002) or ROS levels (Le Belle et al., 2011). Multiple lines of evidence have demonstrated that NSCs express GFAP and that these cells sustainably contribute to neurogenesis in vivo (Doetsch et al., 1997; Doetsch et al., 1999a; Doetsch et al., 1999b; Doetsch et al., 2002; Imura et al., 2003; Morshead et al., 2003; Garcia et al., 2004; Mirzadeh et al., 2008; Pastrana et al., 2009; Beckervordersandforth et al., 2010; Giachino et al., 2013). Pastrana et al. identified quiescent GFAP-GFP⁺EGFR⁻ SVZ cells and speculated that these cells include NSCs that give rise to more mitotically active GFAP-GFP⁺EGFR⁺ and GFAP-GFP⁻EGFR⁺ NICs but did not test this by fate-mapping (Pastrana et al., 2009). In contrast, it has been suggested that nearly all GFAP-GFP⁺CD133⁺ cells are NICs and that these cells are the qNSCs in the SVZ (Beckervordersandforth et al., 2010). Efforts toward prospective identification have therefore generated conflicting results about whether NICs are quiescent or mitotically active in vivo and regarding their relationship to NSCs.

The inventors have identified regulators of CNS stem cell self-renewal based on their ability to regulate SVZ proliferation and neurogenesis in vivo as well as multipotent NIC self-renewal in culture (Molofsky et al., 2003; Molofsky et al., 2006; Nishino et al., 2008; Chuikov et al., 2010). However, an important question that they have not been able to address directly is whether those genes are necessary for NSC maintenance in vivo. Impaired NIC self-renewal in culture may not reflect reduced NSC self-renewal in vivo (Joseph and Morrison, 2005; He et al., 2009). For example, the polycomb transcriptional repressor Bmi-1 is thought to be required for NSC self-renewal (Molofsky et al., 2003; Bruggeman et al., 2005; Molofsky et al., 2005; Zencak et al., 2005; Bruggeman et al., 2007; Fasano et al., 2009). However, these studies were performed in germline knockout mice that generally die within a month after birth (van der Lugt et al., 1994; Jacobs et al., 1999; Lessard and Sauvageau, 2003; Park et al., 2003). Thus, it has not been possible to test whether Bmi-1 is autonomously required by NSCs in the adult brain or whether NSCs differ from NICs in their dependence upon Bmi-1. Methods of identifying and isolating NSC's and NIC's are therefore greatly in need.

### SUMMARY

Thus, in accordance with the present disclosure, there is provided an isolated neurosphere-initiating cell population characterized by expression of:
moderate levels of Glast (˜10² to ˜6×10³ arbitrary expression units as on FIG. 1B), high levels of EGFR (at least ˜10³ arbitrary expression units as on FIG. 1B), high levels of PlexinB2 (at least ˜10³ arbitrary expression units as on FIG. 1B), negative to low levels of CD24 (less than ˜5×10² arbitrary expression units as on FIG. 1B), negative to low levels of O4 and PSA-NCAM (less than ˜10³ arbitrary expression units as on FIG. 1B), and negative for the hematopoietic markers Ter119 and CD45 (less than ˜10² arbitrary expression units as on FIG. 1B).
The cells may be subventricular zone (SVZ) cells, such as from adult mice or fetal humans. The isolated cell population may have at least 20% cells which form neurospheres upon addition to non-adherent cultures. The isolated cell population may have at least 20% cells which form neurospheres that undergo multilineage differentiation in culture. The isolated cell population may divide frequently in the subventricular zone (SVZ) and is short-lived in vivo, may be sensitive to temozolomide (TMZ) treatment, and/or may require Bmi-1 for its maintenance in vivo during adulthood.

In another embodiment, there is provided an isolated neural stem cell population characterized by expression of:
high levels of Glast (at least ˜10⁴ arbitrary expression units as on FIG. 3E), negative to low levels of Epidermal Growth Factor Receptor (EGFR) (less than ˜5×10² arbitrary expression units as on FIG. 3E), moderate levels of PlexinB2 (less than ˜10³ arbitrary expression units as on FIG. 3E), negative to low levels of CD24 (less than ˜10² arbitrary expression units as on FIG. 3E), negative to low levels of O4 and PSA-NCAM (less than ˜10³ arbitrary expression units as on FIG. 3E), and negative for the hematopoietic markers Ter119 and CD45 (less than ˜10² arbitrary expression units as on FIG. 1B); and resistance to temozolomide.
The isolated cell population may be subventricular zone (SVZ) cells, such as those from adult mice or fetal humans. The isolated cell population may be enriched for GFAP⁺ cells relative to unfractionated subventricular zone (SVZ) cells. The isolated cell population may be enriched for cells recombined by GFAP-CreER^{T2} relative to unfractionated subventricular zone (SVZ) cells. The isolated cell population may be enriched with cells fated to give rise to neurosphere-initiating cells in the subventricular zone (SVZ). The isolated cell population may be highly quiescent, and/or may be dependent on Bmi-1 for maintenance of the cell population in vivo during adulthood.

In yet another embodiment, there is provided a method of isolating a neurosphere initiating cell population comprising (a) providing a cell population from adult mouse brain; and (b) selecting cells exhibiting expression of moderate levels of Glast, high levels of Epidermal Growth Factor Receptor (EGFR), high levels of PlexinB2, negative to low levels of CD24, negative to low levels of O4 and PSA-NCAM, and negative for the hematopoietic markers Ter119 and CD45. The method may further comprise culturing cells selected in step (b). The method may also further comprise treating the cells selected in step (b) with temozolomide (TMZ). The cell population may be is comprised of subventricular zone (SVZ) cells, such as from adult mice or fetal humans. The method may further comprise removing debris from enzymatically dissociated SVZ cells prior to step (a). EDTA may be added to the cell population of step (a). The method may further comprise maintaining the pH of the culture medium at a neutral range, such as by keeping said medium in 6% CO₂. The method may also further comprise adding a Rock inhibitor and/or IGF1 to the culture medium. The method may even further comprise preventing aggregation following selection.

In still another embodiment, there is provided a method of isolating a neural stem cell population comprising (a) providing an enzymatically dissociated cell population from adult mouse brain; and (b) selecting cells that exhibit high levels of Glast, negative to low levels of

Epidermal Growth Factor Receptor (EGFR), moderate levels of PlexinB2, negative to low levels of CD24, negative to low levels of O4 and PSA-NCAM, and negative for the hematopoietic markers Ter119 and CD45. The method may further comprise culturing cells selected in step (b), and further may comprise treating the cells selected in step (b) with temozolomide (TMZ). The cell population may be comprised of subventricular zone (SVZ) cells. The method may further comprise removing debris from enzymatically dissociated SVZ cells prior to step (a). EDTA may be added to the cell population of step (a). The method may further comprise maintaining the pH of the culture medium at a neutral range, such as by keeping the medium in 6% CO₂. The method may further comprise adding a Rock inhibitor and/or IGF1 to the culture medium. The method may also further comprise preventing aggregation following selection.

In an even further embodiment, there is provided a kit comprising antibodies binding immunologically to Glast, Epidermal Growth Factor Receptor (EGFR), PlexinB2, CD24, O4, PSA-NCAM, Ter119 and CD45. The kit may further comprise temozolomide (TMZ). The antibodies may be each attached to a unique label. The kit may also further comprise one or more additional antibodies that bind to a cell surface antigen, such as antibodies selected from anti-CD133 and anti-GFAP. The kit may further comprise instructions for performing flow cytometry with said antibodies, and or for performing culturing of cells following selection with said antibodies. The kit may further comprise Rock inhibitor and/or IGF1, may further comprise one or more buffers, diluents, enzymes, or excipients including HBSS, L15, bovine serum albumin, EDTA, trypsin, DNase, and trypsin inhibitor solution, and/or may further comprise cell culture medium components including Dulbecco's Modified Eagle Medium, Neurobasal medium, B-27 and N-2 supplements, chick embryo extract, beta-mercaptoethanol, penicillin-streptomycin, and epidermal and fibroblast growth factors.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-E: Prospective identification and isolation of neurosphere-initiating cells. (FIG. 1A) A screen of antibodies identified cell surface markers of NICs in the adult mouse SVZ. (FIG. 1B) Flow cytometric gating strategy to isolate GEPCOT cells (Glast^{mid}Egfr^{high}PlexinB2^{high}CD24^{-/low}O4/PSA-NCAM^{-/low}Ter119/CD45⁻) that represent 3.2±0.7% of young adult mouse SVZ cells. Plots represent one representative experiment from at least nine independent experiments. For more information on gating see FIGS. 9A-C. (FIG. 1C) Frequency of all neurospheres (>50 μm) and multipotent neurospheres formed by unfractionated SVZ cells (DAPI/CD45/Ter119⁻), GEPCOT cells, and remaining live SVZ cells outside of the GEPCOT population (n=9 independent experiments). (FIG. 1D) Frequency of BrdU⁺ SVZ cells or GEPCOT cells after BrdU pulses in vivo (n=5-11 mice/time point in 2-3 independent experiments). (FIG. 1E) GEPCOT cells efficiently formed multipotent neurospheres in vitro and were mitotically active in vivo. Data always represent mean ±s.d. Statistical significance was assessed with two-tailed t-tests, ***p <0.001.

FIGS. 2A-G: Individual NICs persist only transiently within the SVZ in vivo but are constantly replenished by more primitive neural stem cells. (FIG. 2A) Mice bearing inducible Cre alleles and the loxp-tdTomato conditional reporter were induced with five consecutive days of tamoxifen injections (80 mg/kg/day) then chased for 2 to 60 days before analysis of the percentage of labeled SVZ cells or GEPCOT cells in vivo or the percentage of labeled neurospheres (>50 μm) in culture. (FIGS. 2B-F) Cells marked by recombination of Dlx1^{CreERT2} (FIG. 2B, n=4-7 mice/time point in 4 independent experiments), Ascl1^{CreERT2} (FIG. 2C, n=3 or 4 mice/time point in 3 independent experiments), Gli1^{CreERT2} (FIG. 2D), Glast-CreER^{T} (FIG. 2E), or Sox2^{CreERT2} (FIG. 2F, n=3-4 mice/time point in 4 independent experiments for FIGS. 2D-F). (FIG. 2G) A model consistent with the fate mapping data involving a NSC population that gives rise to a transient NIC population. Data represent mean ±s.d. Statistical significance was tested among sequential days of analysis (7 d was compared to 2 d, 28 d was compared to 7 d, and 60 d was compared to 28 d) with a one-way ANOVA followed by Tukey's post-hoc tests for multiple comparisons, *p <0.05, **p <0.01, ***p <0.001, n.s. not significant.

FIGS. 3A-E: Identification of a pre-GEPCOT population that gives rise to GEPCOT NICs in vivo. (FIG. 3A) GFAP-CreER^{T2}; loxp-tdTomato mice were induced with tamoxifen for 5 days, chased for 2 to 60 days without tamoxifen, then conditional reporter expression was analyzed in all SVZ cells, GEPCOTs, and cultured neurospheres (n=3-5 mice/time point in 4 independent experiments). (FIGS. 3B-D) GFAP-CreER^{T2}; loxp-tdTomato mice were analyzed by flow cytometry to quantify reporter expression in SVZ cells without tamoxifen (FIG. 3B), at 2 days (FIG. 3C) or 28 days (FIG. 3D) after tamoxifen treatment. (FIG. 3B) Without tamoxifen, virtually no SVZ cells expressed the reporter. For comparison to FIG. 3C and FIG. 3D, control SVZ cells are shown stained for Glast and EGFR as well as the distribution of the Glast^{high}EGFR^{-/low} fraction with respect to PlexinB2 and O4/PSA-NCAM staining. (FIG. 3C) Two days after tamoxifen, the tdTomato⁺ cells labeled by recombination by the GFAP-CreER^{T2} stem cell marker were Glast^{high}Egfr^{-/low}PlexinB2^{mid}CD24^{-/low}O4/PSA-NCAM^{-/low}Ter119/Cd45⁻ (CD24 expression is not shown here), which the inventors describe as ‘pre-GEPCOT’ cells. (FIG. 3D) By 28 days after tamoxifen, Glast^{mid}Egfr^{high}PlexinB2^{high}CD24^{-/low}O4/PSA-NCAM^{-/low}Ter119/CD45⁻ GEPCOT cells were also labeled. (FIG. 3E) The gating strategy to identify the GFAP-expressing pre-GEPCOT population that comprises 6±3% of SVZ cells. Statistical significance was tested among sequential days of analysis with a one-way ANOVA followed by Tukey's post-hoc tests for multiple comparisons. *p <0.05, **p<0.01, *** p <0.001, n.s. not significant.

FIGS. 4A-M: GFAP-expressing pre-GEPCOT cells are quiescent in vivo but make an enduring contribution to the SVZ. (FIG. 4A) Antibody staining for GFAP among SVZ cells, GEPCOTs, and pre-GEPCOTs (scale bar=5 μm) (n=3 mice from 3 independent experiments). (FIG. 4B) The frequencies of neurospheres >50 μm, multipotent neurospheres, adherent colonies, and multipotent adherent colonies (4-6 independent experiments each) formed by unfractionated live SVZ cells, GEPCOTs, and pre-GEPCOTs. (FIG. 4C) Frequency of BrdU⁺ SVZ cells or pre-GEPCOT cells after BrdU pulses in vivo (n=5-11 mice/time point in 2-3 independent experiments; note that SVZ data are from FIG. 1D for comparison purposes but were obtained in the same experiments). (FIGS. 4D-H) Conditional reporter expression in pre-GEPCOT cells at varying times after recombination with the indicated Cre alleles. These data are from the same fate-mapping experiments as shown in FIGS. 2A-G and FIGS. 3A-E, including the same SVZ data for comparison purposes. Cre alleles that only transiently contributed to the SVZ (Dlx1^{CreERT2} and Ascl1^{CreERT2}) did not recombine in pre-GEPCOT cells, while Cre alleles that gave enduring contributions to the SVZ (Gli1^{CreERT2}, Glast-CreER^{T}, and Sox2^{CreERT2}) did recombine in pre-GEPCOT cells. (FIGS. 4I-J) The frequencies and numbers of labeled pre-GEPCOT cells, GEPCOT cells, neuroblasts, and other SVZ cells at varying times after recombination by GFAP-CreER^{T2} (n=3-5 mice/time point in 4 independent experiments). (FIG. 4K) Markers that distinguish pre-GEPCOT from GEPCOT cells. (FIGS. 4L-M) Whole-mount SVZs were stained with anti-acetylated tubulin (red), anti-β-catenin (blue), and either anti-GFAP (FIG. 4L, green) or anti-EGFR (M, green) antibodies and pinwheel structures were inspected for the presence of GFAP^{high} pre-GEPCOT cells (open arrow in FIG. 4L and FIG. 4M) and EGFR^{high} GEPCOT cells (hatched arrow in M) by confocal microscopy. Images were taken at the apical surface except the GFAP depth projection which is a composite of 11 images at 2 nm intervals into the tissue. Scale bar=5 μm. All data represent mean ±s.d. Statistical significance of differences between SVZ and pre-GEPCOT cells in C was assessed with two-tailed student's t-tests. Statistical significance of differences in FIGS. 4D-J (among time points) was tested with a one-way ANOVA followed by Tukey's post-hoc tests for multiple comparisons. *p <0.05, **p<0.01, ***p <0.001, n.s. not significant.

FIGS. 5A-P: Treatment with temozolomide does not affect the frequency of pre-GEPCOT cells but ablates GEPCOT NICs. (FIGS. 5A-F) Mice were injected with TMZ (100 mg/kg/day) for 3 consecutive days to ablate dividing cells, then allowed to recover for 3 to 90 days before analysis (TMZ 1×). All TMZ 1× data reflect 5-7 mice per time point from 7 independent experiments. (FIGS. 5G-L) Alternatively, mice were serially treated with two doses of TMZ 12 days apart then allowed to recover for 3 to 90 days before analysis (TMZ 2×). All TMZ 2× data reflect 4-9 mice per time point from 6 independent experiments. At each time point after TMZ treatment the panels show the total number of cells isolated per SVZ (FIGS. 5B and 5H), the number of SVZ cells per section that incorporated a 2 hour pulse of BrdU (FIGS. 5C and 5I), the number of multipotent neurospheres that arose in culture per SVZ (FIGS. 5D and 5J), the number of GEPCOTs per SVZ (FIGS. 5E and 5K), and the number of pre-GEPCOTs per SVZ (FIGS. 5F and 5L). (FIGS. 5M-P) Mice were injected with tamoxifen (80 mg/kg/day i.p.) for five days, then with TMZ (100 mg/kg/day i.p.) for three days, then recovered for 3 or 35 days to observe regeneration. The numbers of labeled GEPCOTs were measured after recombination with Ascl1^{CreERT2} (FIGS. 5N, 4 mice per condition from 2 independent experiments), Glast-CreER^{T} (FIGS. 5O, 5-6 mice per condition from 3 independent experiments), or GFAP-CreER^{T2} (FIGS. 5P, 4 mice per condition from 2 independent experiments). All data represent mean ±s.d. Statistical significance was tested with a one-way ANOVA followed by Sidak's post-hoc test for the indicated comparisons in FIGS. 5B-F and FIGS. 5H-L. Statistical significance in N-P was assessed with two-tailed student's t-tests. *p <0.05, **p<0.01, ***p <0.001, n.s. not significant.

FIGS. 6A-O: Reduced neurogenesis, SVZ cell proliferation, GEPCOT frequency, and pre-GEPCOT frequency in Nestin-Cre; Bmi-1^{fl/fl} adult mice. (FIG. 6A) Bmi-1 transcript levels in GEPCOT and pre-GEPCOT cells, normalized to β-actin and shown relative to SVZ cells (n=5 mice in 2 independent experiments). (FIG. 6B) Western blot of

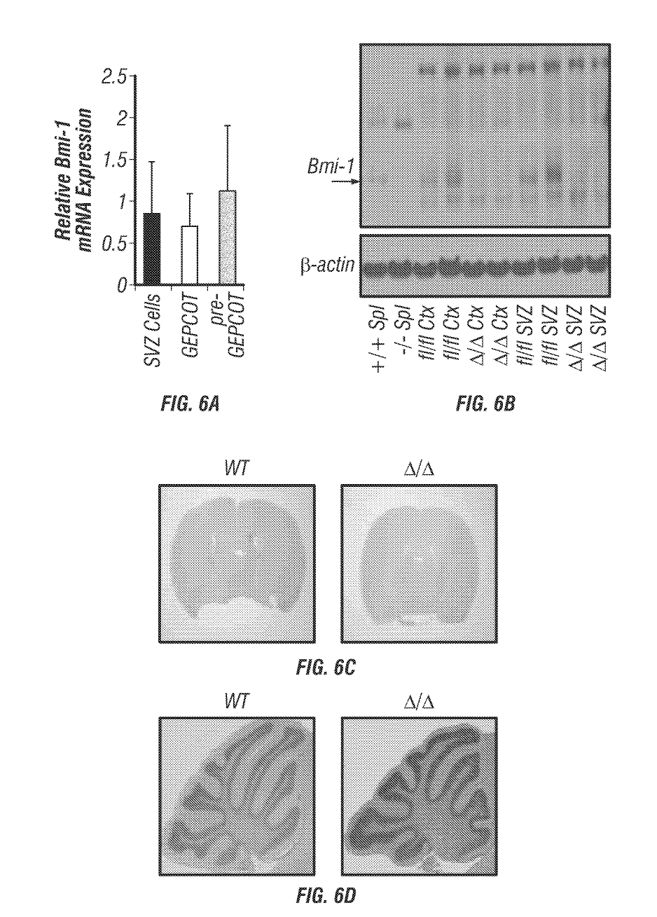

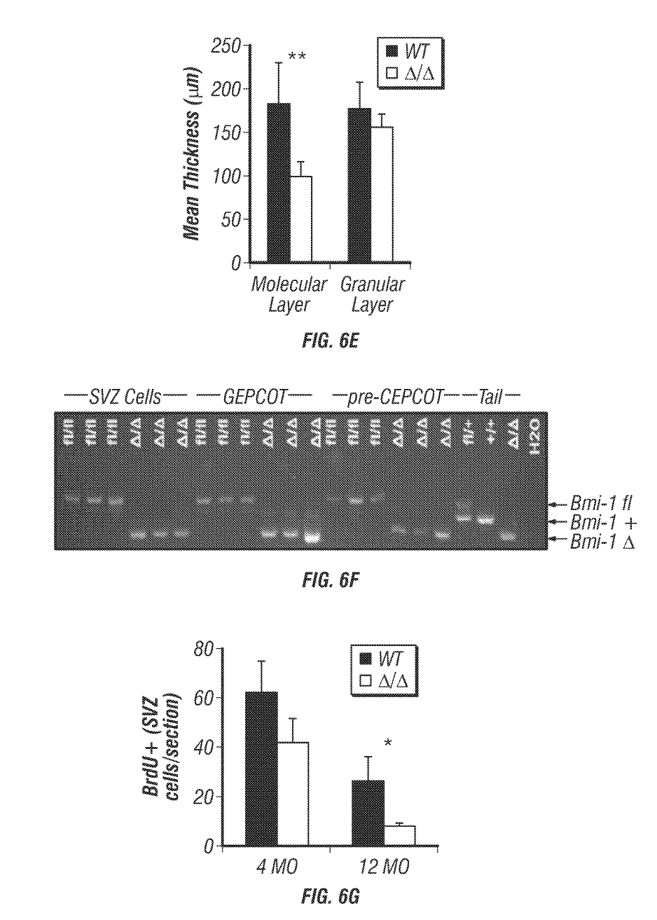

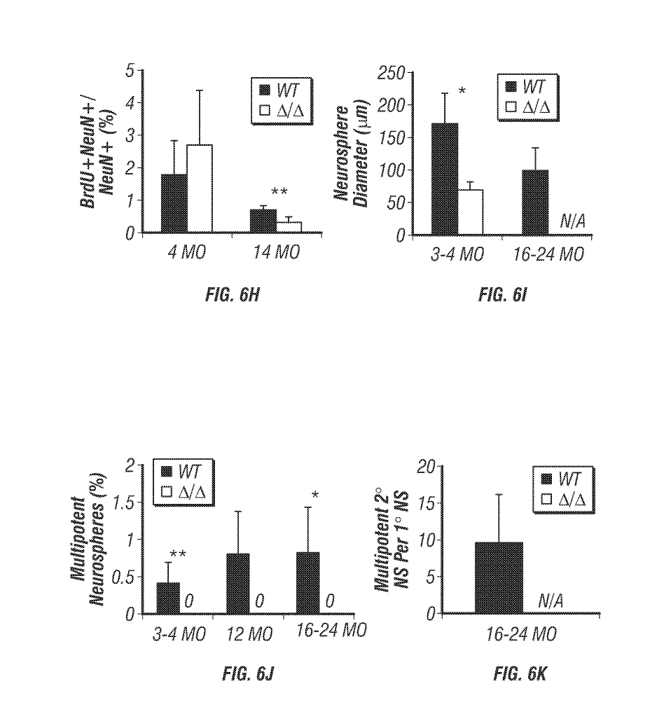

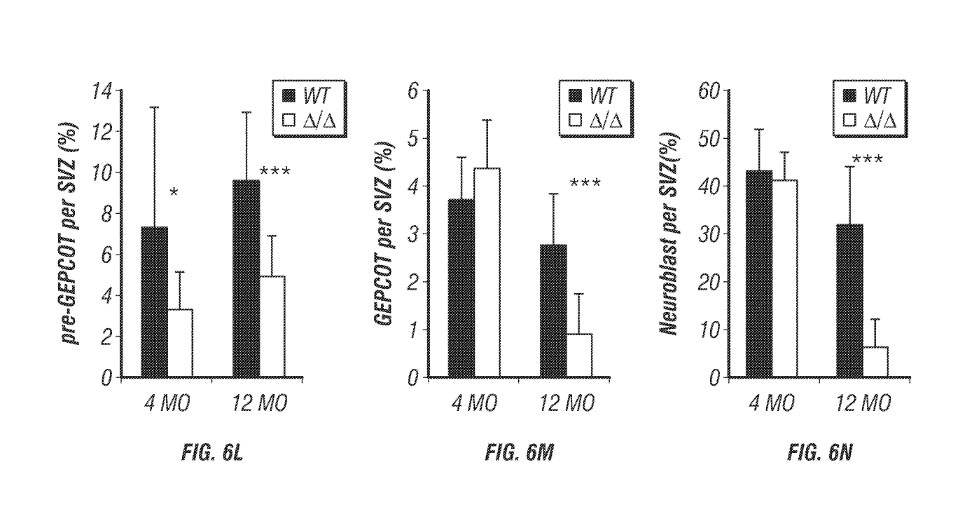

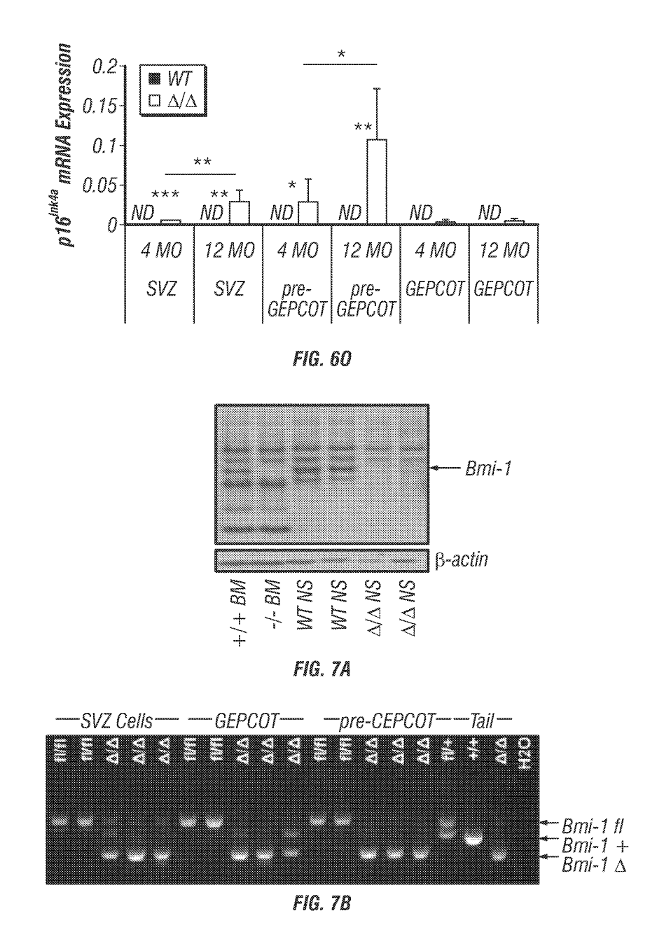

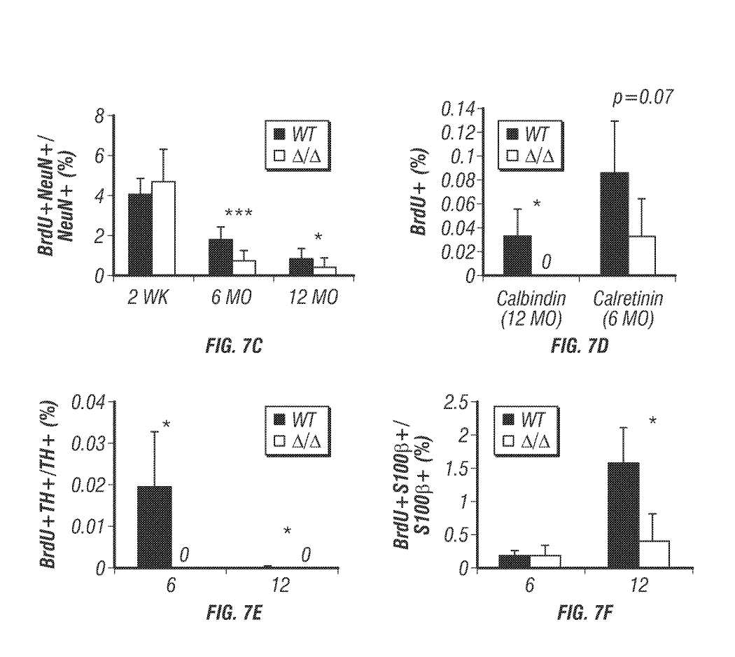

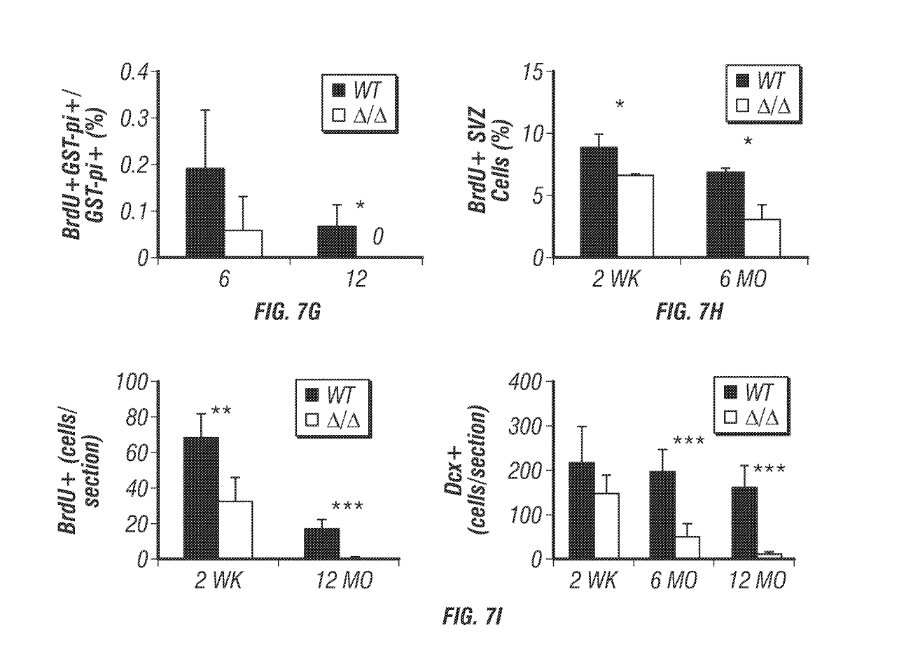

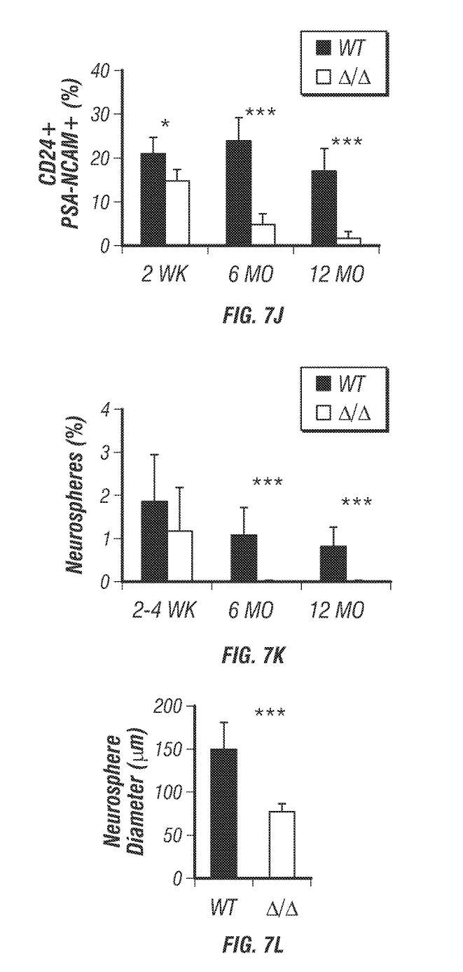

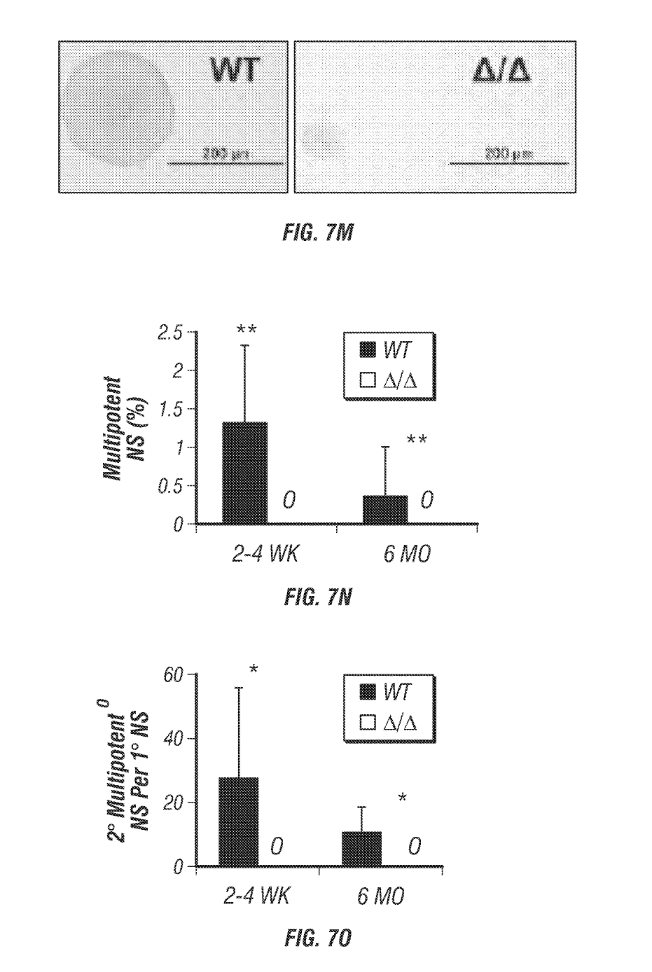

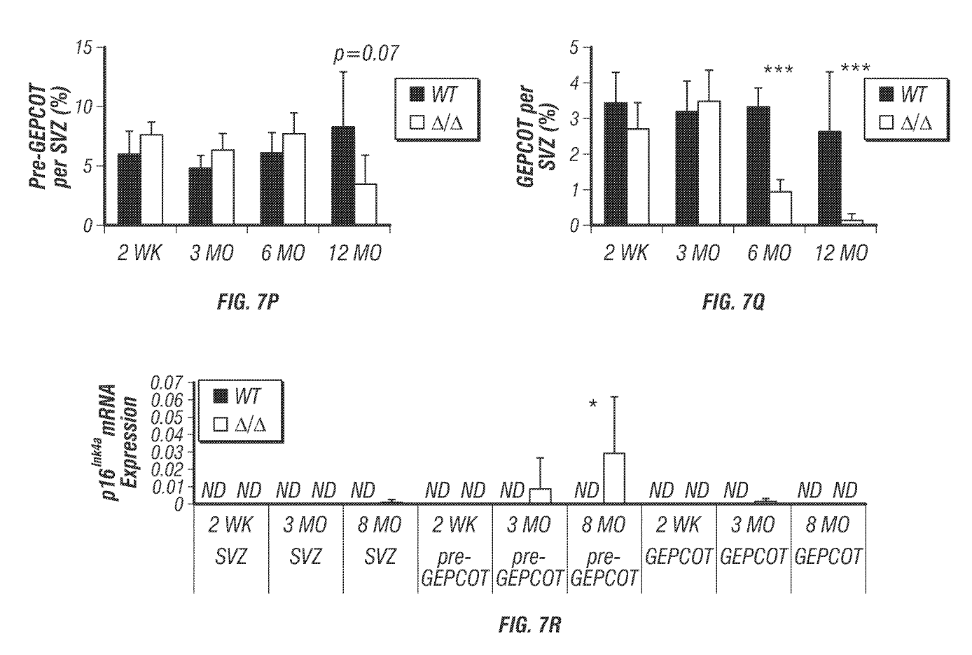

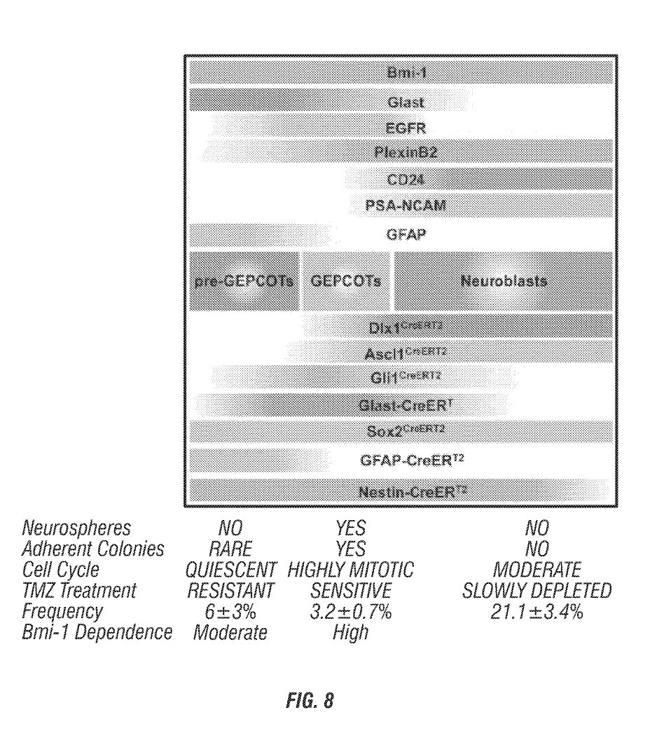

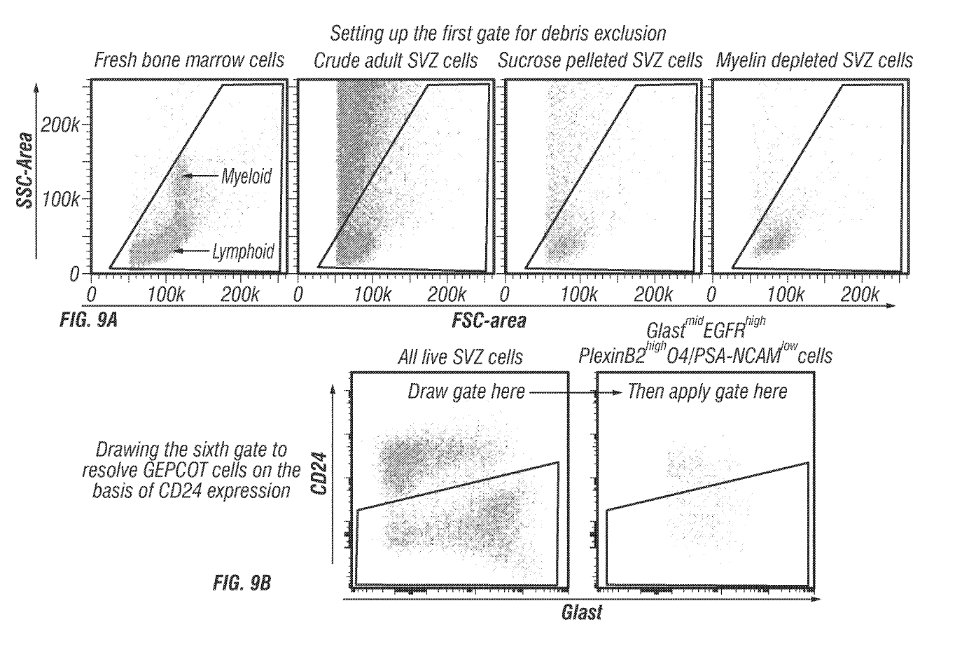

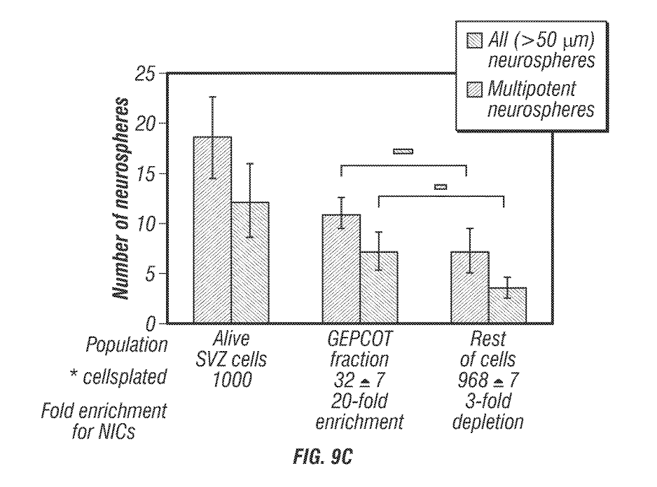

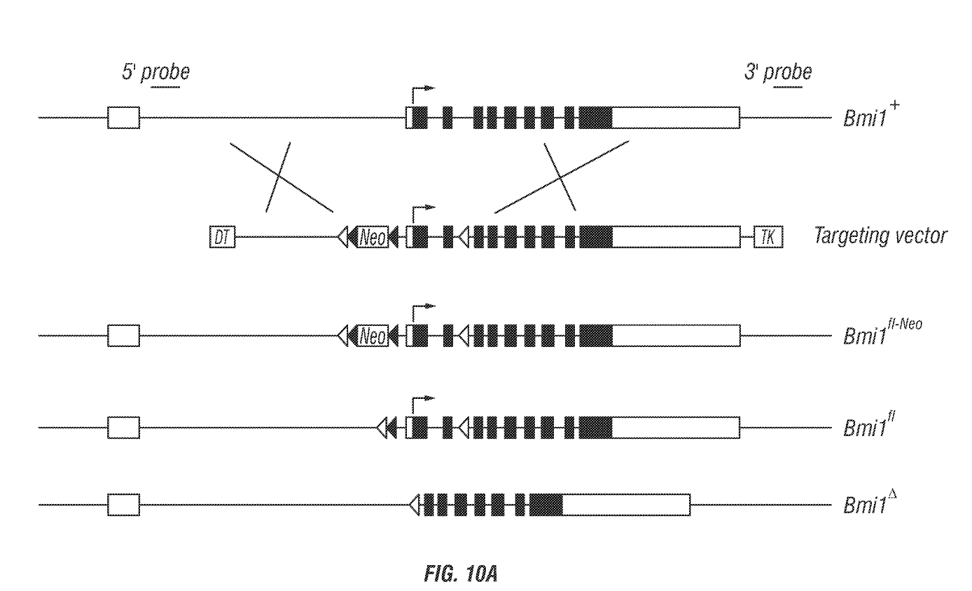

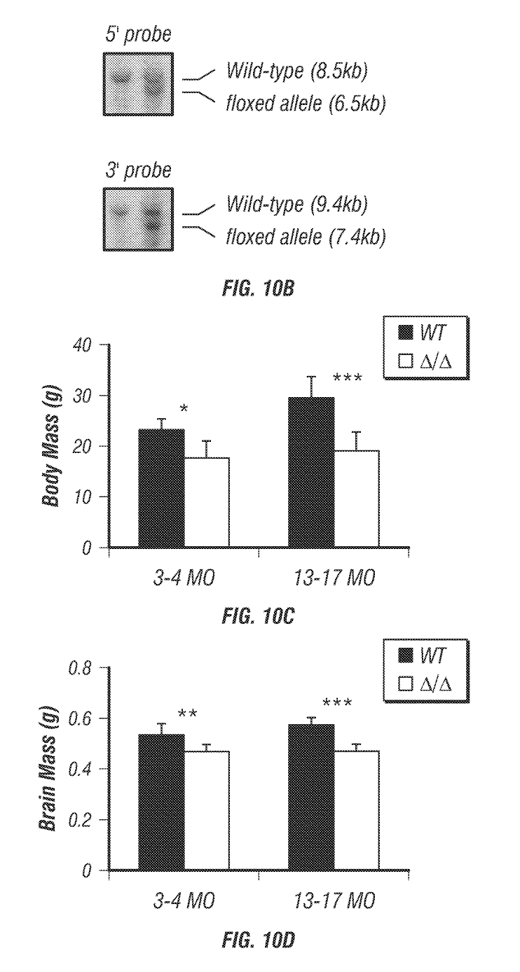

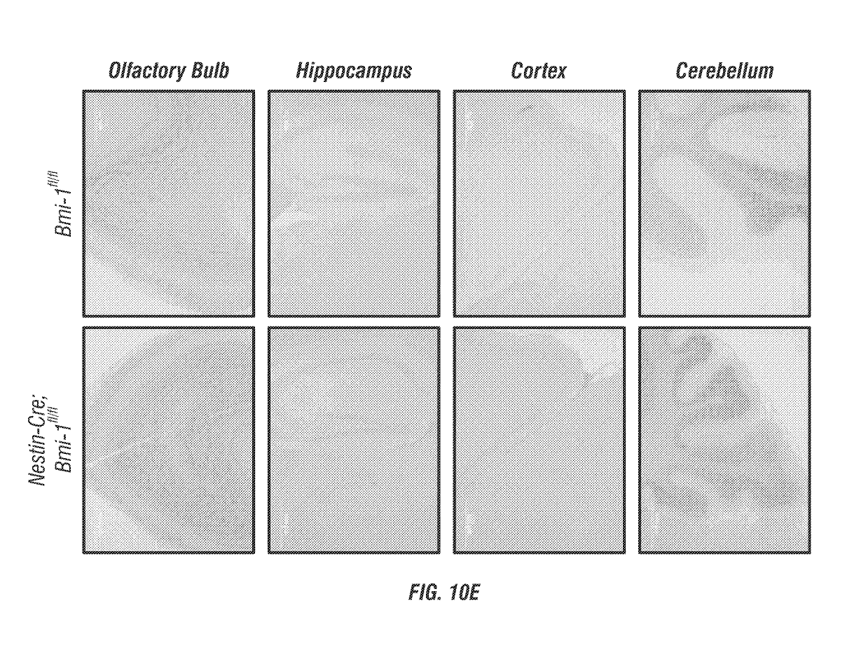

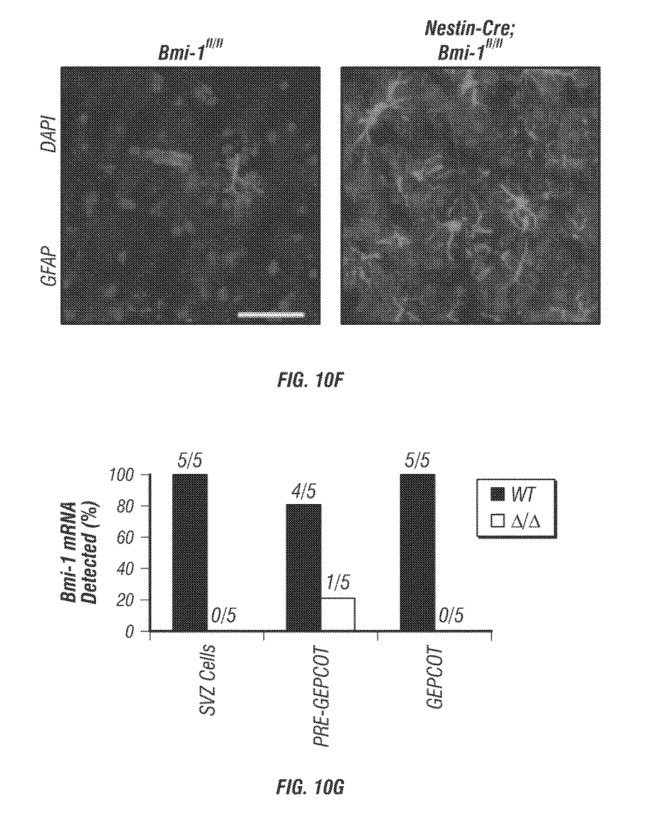

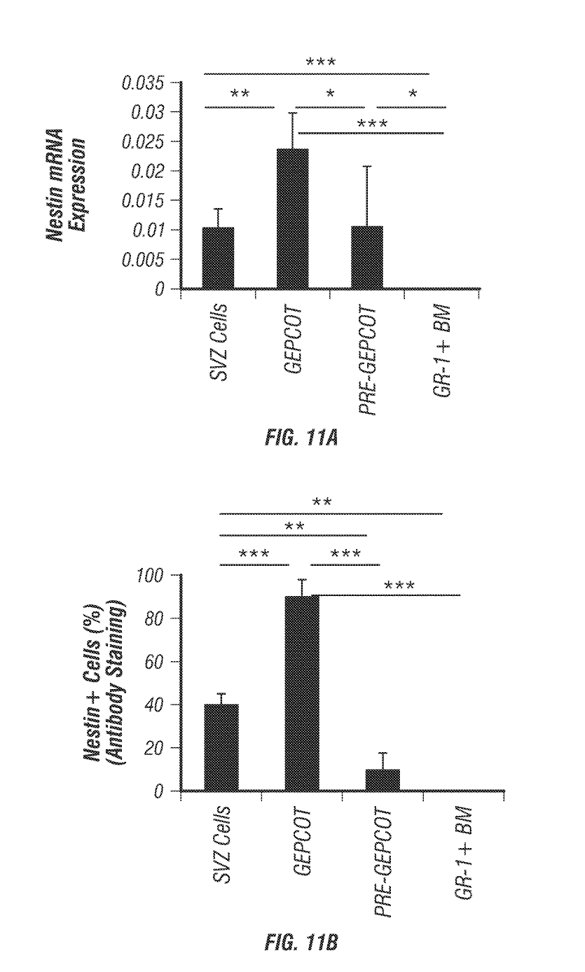

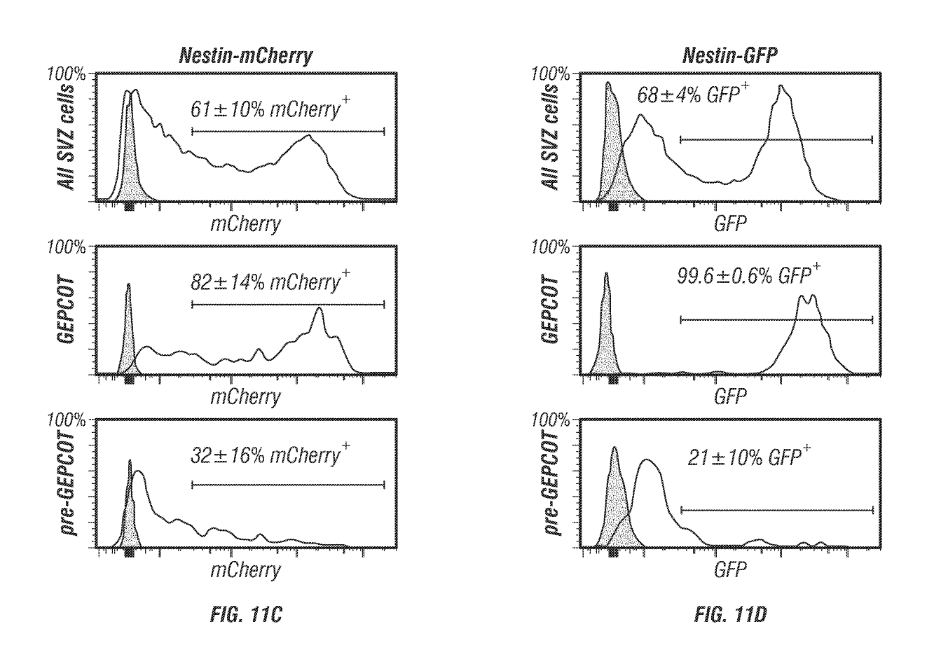

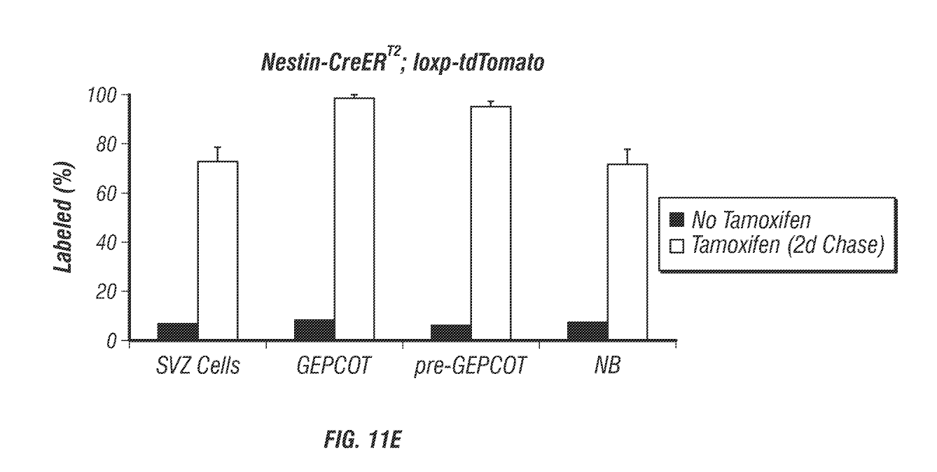

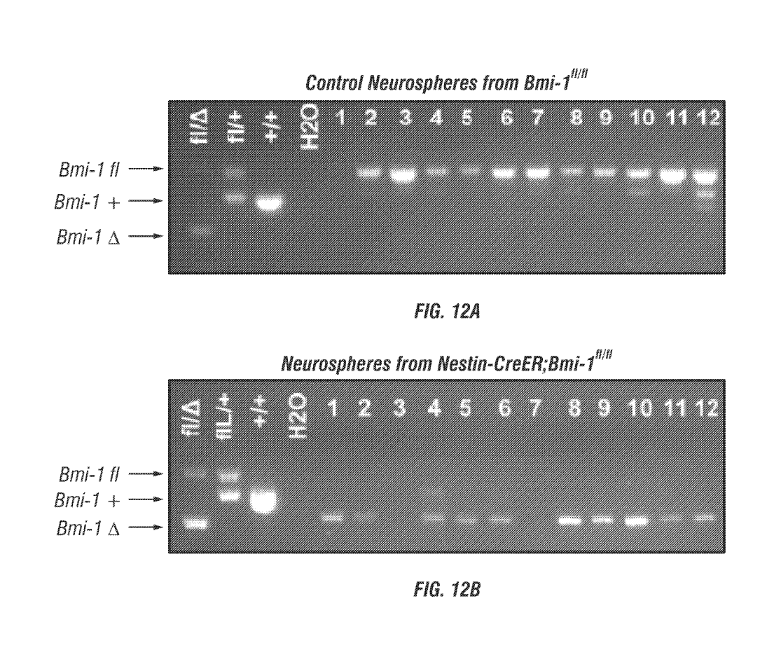

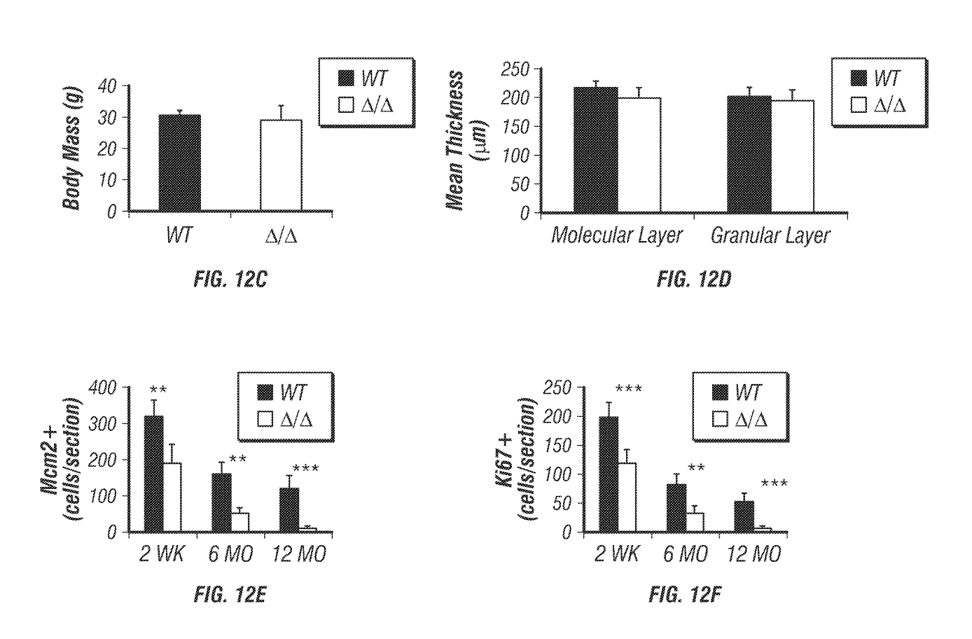

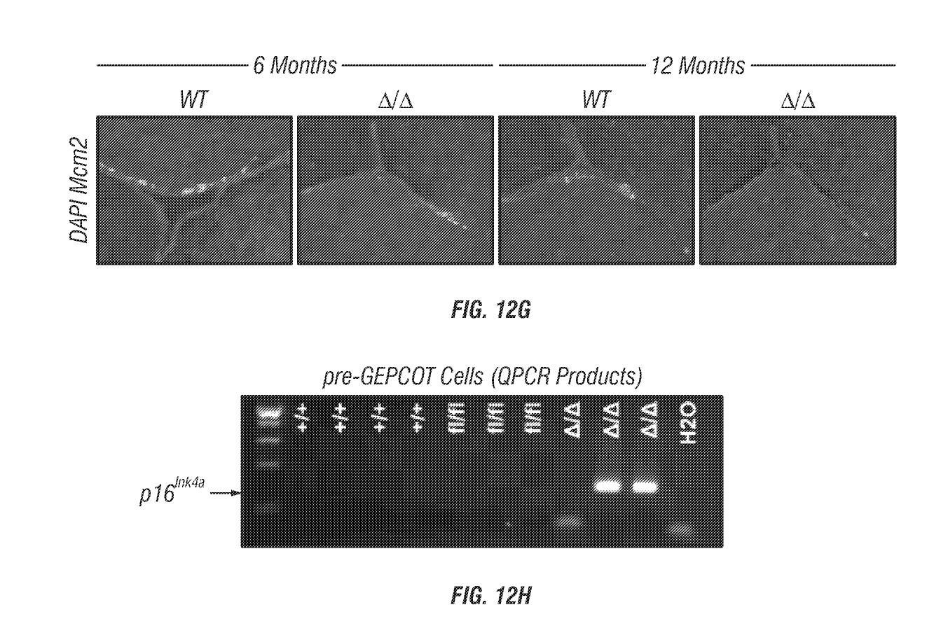

SVZ cells and cortical (Ctx) cells from adult Nestin-Cre; Bmi-^{fl/fl} (Δ/Δ, n=2) mice and Bmi-1^{fl/fl} (fl/fl, n=2) controls. One of 2 independent blots is shown. Splenocytes (Spl) from wild-type (+/+) and germline Bmi-1 deficient (−/'1) mice are shown as controls. (FIGS. 6C-D) Representative images of the forebrain (FIG. 6C) and cerebellum (FIG. 6D). (FIG. 6E) Cerebellar molecular and granular layer thickness (n=3-6 mice/genotype with >8 measurements from 2 sections/mouse). (FIG. 6F) PCR analysis of genomic DNA from SVZ cells, GEPCOT cells, and pre-GEPCOT cells (3 mice/genotype, 1 of 2 independent experiments) as compared to controls (tail DNA and water). A faint non-specific band is visible in Δ/Δ GEPCOT cells. (FIG. 6G) The number of BrdU⁺ SVZ cells per section after a 2-hour pulse of BrdU (n=3 mice/genotype/age, >6 sections per mouse). (FIG. 6H) The frequency of newborn olfactory bulb BrdU⁺NeuN⁺ neurons.after BrdU administration for 7 days followed by 4 weeks without BrdU (n=3-6 mice/genotype/age in 2 independent experiments). (FIG. 6I) The diameter of primary neurospheres (n=3-4 mice/genotype/age in 4 independent experiments. N/A: no neurospheres were formed). (FIG. 6J) The frequency of SVZ cells that formed multipotent neurospheres (n=4-7 mice/genotype/age in 4 independent experiments). (FIG. 6K) The number of secondary neurospheres generated upon subcloning of individual neurospheres (n=6 neurospheres/mouse for 3 WT mice). (FIG. 6L-N) The frequency of (FIG. 6L) pre-GEPCOT cells, (FIG. 6M) GEPCOT cells and (FIG. 6N) neuroblasts in the SVZ of 4- and 12-month-old mice (n=13 mice/genotype/age in 3 independent experiments). (FIG. 6O) p16^{Ink4a} mRNA expression normalized to β-actin in SVZ, pre-GEPCOT and GEPCOT cells (n=5 mice/genotype/age). p16^{Ink4a} was undetectable in all Bmi/1^{fl/fl} samples. In Nestin-Cre; Bmi-1^{fl/fl} mutants p 16^{Ink4a} was detected in SVZ cells (5/5 4-month-old; 5/5 12-month-old) and pre-GEPCOT cells (4/5 4-month-old; 5/5 12-month-old) but usually not in GEPCOT cells (2/5 4-month-old; 1/5 12-month-old). Data represent mean ±s.d. In FIGS. 6C-E and FIGS. 6G-O, WT is Bmi-1^{f/fl} and Δ/Δ is Nestin-Cre; Bmi-1^{fl/fl} adult mice, and times are ages of mice. Statistical significance was assessed by two-tailed student's t-tests. *p <0.05, **p <0.01, ***p <0.001.

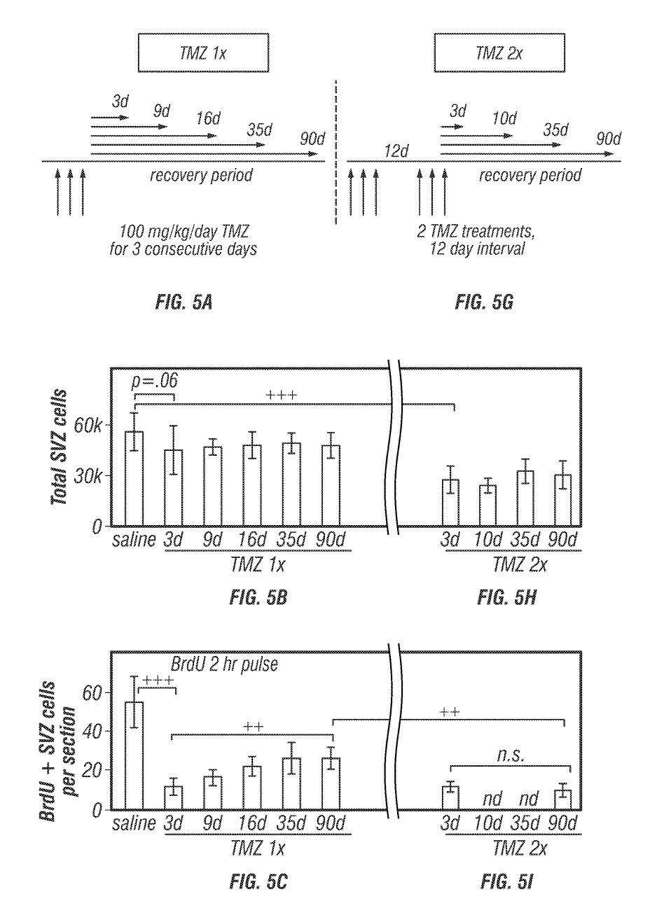

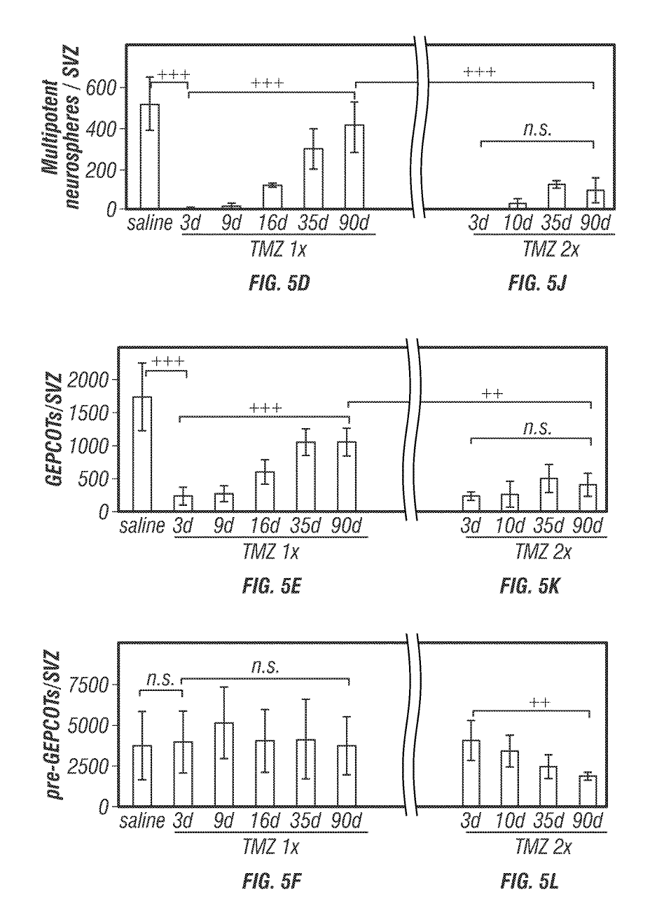

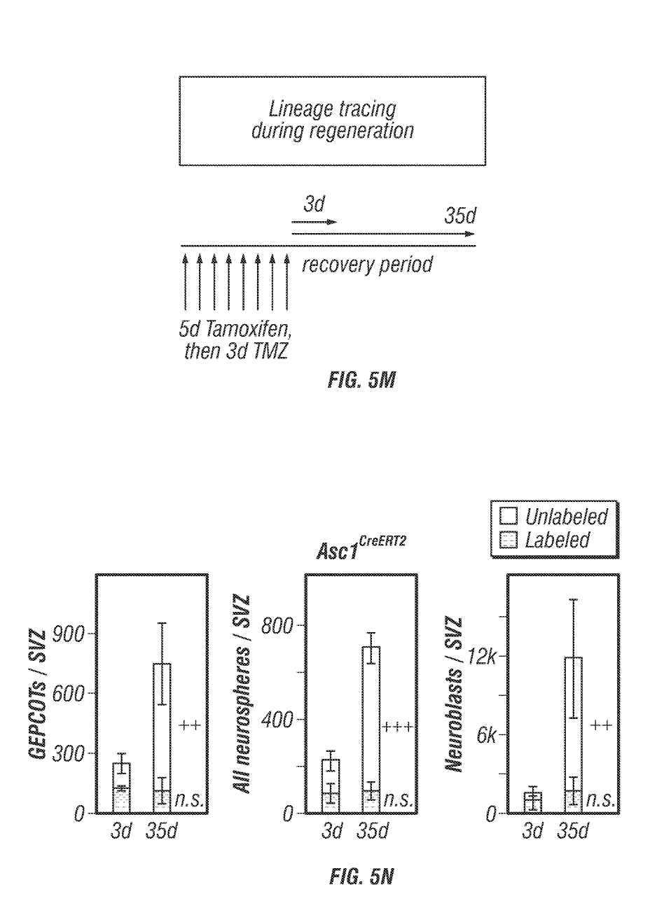

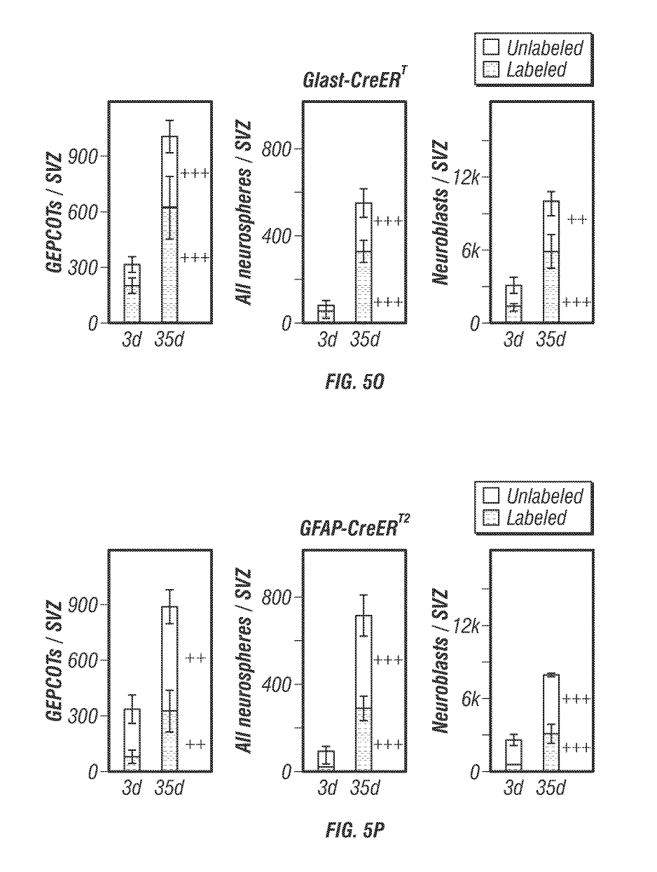

FIGS. 7A-R: Reduced neurogenesis, gliogenesis, SVZ cell proliferation, and GEPCOT NICs in adult Nestin-CreER^{T2}; Bmi-1^{fl/fl} mice relative to littermate controls. (FIG. 7A) Western blot of pooled neurospheres (NS) cultured at 2 weeks after tamoxifen treatment (n=2 mice/genotype). Wild-type and germline Bmi-1^{-/-} bone marrow (BM) cells are shown as controls. (FIG. 7B) PCR analysis of genomic DNA from SVZ cells, GEPCOT cells, and pre-GEPCOT cells one day after tamoxifen treatment as compared to controls (tail DNA and water) (n=2-3 mice/genotype). A faint non-specific product is visible in lanes 3, 8 and 10. (FIG. 7C) The frequency of newborn BrdU⁺NeuN⁺ neurons in the olfactory bulb (n=5-11 mice/genotype/timepoint in 10 independent experiments). (FIGS. 7D-G) The frequencies of newborn BrdU⁺ Calbindin⁺ (FIG. 7D), Calretinin⁺ (FIG. 7D), Tyrosine Hydroxylase⁺ (FIG. 7E), and S100β⁺ (FIG. 7F) cells in the olfactory bulb or GST-pi⁺ (FIG. 7G) cells in the cortex (n=3-5 mice/genotype/timepoint in 2 independent experiments). For FIGS. 5C-G BrdU was administered for 7 days followed by 4 weeks without BrdU. (FIG. 7H) The frequency (left panel) or number (right panel) of BrdU⁺ SVZ cells per section after a 2-hour pulse of BrdU (n=3-11 mice/genotype/timepoint in 6 independent experiments). (FIGS. 7I-J) The number of Dcx⁺ neuroblasts per section (FIG. 7I) or frequency of CD24^{mid}PSA-NCAM⁺ neuroblasts as determined by flow cytometry (n=4-11 mice/genotype/age in 6-7 independent experiments). (FIGS. 7K, 7N) The frequencies of SVZ cells that formed neurospheres (>50 μm; FIG. 7K), or multipotent neurospheres (FIG. 7N; n=5-9 mice/genotype/timepoint in 6 independent experiments). (FIG. 7L) The diameter of primary neurospheres from mice 2-4 weeks after tamoxifen treatment (n=7-9 mice/genotype in 2 independent experiments). (FIG. 7M) Representative primary neurospheres from mice 2 weeks after tamoxifen treatment. (FIG. 7O) The number of multipotent secondary neurospheres generated upon subcloning of individual primary neurospheres (n=3-9 mice/genotype/timepoint in 3 independent experiments). (FIG. 7P-Q) The frequency of pre-GEPCOT cells (FIG. 7P) or GEPCOT cells (FIG. 7Q) in the SVZ (n=3-11 mice/genotype/timepoint in 7 independent experiments). (FIG. 7R) qRT-PCR analysis of p16^{Ink4a} transcript levels expression normalized to β-actin in SVZ, pre-GEPCOT and GEPCOT cells (n=5-9 mice/genotype/timepoint). All data represent mean ±s.d. In A and C-R, WT is Bmi-1^{fl/fl} and Δ/Δ is Nestin-CreER^{T2}; Bmi-1^{fl/fl} adult mice, and timepoints are times after tamoxifen induction beginning at 6 weeks of age. Statistical significance was assessed with two-tailed student's t-tests. *p <0.05, ** p <0.01, *** p <0.001.

FIG. 8: Phenotypically and functionally distinct populations of pre-GEPCOT qNSCs and GEPCOT NICs. Glast^{high}EGFR^{-/low}PlexinB2^{mid}CD24^{-/low}O4/PSA-NCAM^{-/low}Ter119/CD45⁻ pre-GEPCOT cells are a quiescent, TMZ-resistant population containing qNSCs that give rise to Glast^{mid}EGFR^{high}PlexinB2^{high}CD24^{-/low}O4/PSA-NCAM^{-/low}Ter119/CD45⁻ GEPCOT cells that are highly mitotically active in vivo and enriched for NICs. pre-GEPCOT cells include Type B1 cells, GEPCOT cells include Type C cells, and neuroblasts make up Type A SVZ cells (Doetsch et al., 1997; Doetsch et al., 1999a). Bars for different Cre alleles represent the extent of recombination observed after 5 consecutive days of tamoxifen treatment followed by a 2 day chase.

FIGS. 9A-C: Isolating GEPCOT cells by flow cytometry. (FIG. 9A) Side scatter (SSC) and forward scatter (FSC) were gated to eliminate debris. The parameters of this gate can be set using mouse bone marrow as shown in the first panel such that myeloid cells fall in the center of the plot. FSC threshold was increased to eliminate as much debris as possible without eliminating cells (this can be checked by sorting events onto a microscope slide and then checking by microscopy to determine whether the events include cells or debris). These steps ensure reproducibility across days in spite of extensive debris in SVZ cell preparations. Sucrose density centrifugation (approximately 90% debris reduction) or myelin depletion using paramagnetic myelin-binding microbeads (Miltenyi Biotec, 130-096-733, approximately 95-98% debris reduction) greatly reduce debris but also reduce cell yield by ˜50-60%. (FIG. 9B) The sixth gate (CD24/Glast) is drawn using the ‘live SVZ cells’ population as a guide. On a plot of Glast versus CD24, live SVZ cells should have two clearly resolved populations that are either CD24^{mid} or CD24^{-/low}. The CD24 gate excludes all the CD24^{mid} cells and retains all the CD24^{-/low} cells. (FIG. 9C) Most NICs and multipotent NICs were contained in the GEPCOT population, which was 20-fold enriched for NICs relative to unfractionated SVZ cells. The remaining non-GEPCOT cells were 3-fold depleted for NICs relative to unfractionated SVZ cells. Data represent mean ±s.d from 11 (neurospheres >50 μm) or 5 (multipotent neurospheres) independent experiments. Significance was assessed using two-tailed t-tests (**, p <0.01; ***, p <0.001).

FIGS. 10A-G: Targeting strategy for engineering a floxed allele of Bmi-1 for conditional deletion. (FIG. 10A) A bacterial artificial chromosome containing the mouse Bmi-1 gene (RP23-14513), was modified to insert a loxP-FRT-Neo-FRT cassette 5′ to exon 2 and a loxP site 3′ to exon 3 to generate the targeting vector. These sites were selected to avoid disrupting conserved sequences, which are potential regulatory elements. Correctly targeted ES cells (Bmi1^{fl-Neo}) were generated using W4 ES cells and identified by southern blotting (FIG. 10B) using 5′ and 3′ probes. After generating chimeric mice that gave germline transmission, Bmi1^{fl-Neo} mice were mated with Flp deleter mice (Rodriguez et al., 2000) to remove the Neo cassette. The resulting Bmi-1^{fl} mice were backcrossed for at least 10 generations onto a C57BL background. Cre recombination of this allele deletes the start codon and generates a frameshift mutation. Open boxes indicate non-coding sequences while black boxes indicate coding sequences. Open and black triangles indicate loxP and FRT sites, respectively. (FIGS. 10C-D) Body mass (FIG. 10C; n=4-13 mice/genotype/age) and brain mass (FIG. 10D; n=5-17 mice/genotype/age) of Nestin-Cre; Bmi-1^{fl/fl} mice and Bmi-1^{fl/fl} controls. (FIG. 10E) Representative images from different regions of the brains of Nestin-Cre; Bmi-1^{fl/fl} mice and Bmi-1^{fl/fl} controls. (FIG. 10F) Representative images from the cortex of Nestin-Cre; Bmi-1^{fl/fl} mice and Bmi-1^{fl/fl} controls stained for GFAP and DAPI (scale bar equals 50 μm). (FIG. 10G) Fraction of mice that exhibited detectable Bmi-1 transcript levels by RT-PCR in SVZ, pre-GEPCOT and GEPCOT cells from 4 month-old Nestin-Cre; Bmi-1^{fl/fl} mice and Bmi-1^{fl/fl} controls (n=5 mice/genotype). Data represent mean ±s.d. Statistical significance was assessed with two-tailed student's t-tests (*p <0.05, ** p<0.01, *** p <0.001).

FIG. 11A-E: Nestin expression in GEPCOT and pre-GEPCOT cells. (FIG. 11A) Nestin transcript expression relative to β-actin (n=5 mice in 2 independent experiments). (FIG. 11B) Frequency of cells within each cell population that stained positively for Nestin protein (n>50 cells counted/population in each of 3 independent experiments). (FIGS. 11C-D) Representative histograms of Nestin-mCherry (FIG. 11C) and Nestin-GFP (FIG. 11D) transgene expression in SVZ cells, GEPCOT cells, and pre-GEPCOT cells. Shaded histograms represent background fluorescence in wild-type controls (mean±s.d. reflects data from 3 mice per genotype in a single experiment). (FIG. 11E) Nestin-CreER^{T2}; loxp-tdTomato mice were treated with five consecutive days of tamoxifen injections (80 mg/kg/day) then chased for 2 days (n=4 mice). The frequency of tdTomato+cells in each cell population is shown. Untreated mice reflect the background level of recombination (n=2 mice). Data represent mean ±s.d. Statistical significance was assessed with two-tailed student's t-tests (*p <0.05, ** p<0.01, *** p <0.001).

FIGS. 12A-H: Adult Nestin-CreER^{T2}; Bmi-1^{fl/fl} mice show Bmi-1 recombination in pre-GEPCOT qNSCs. (FIGS. 12A-B) PCR of genomic DNA from individual neurospheres cultured from Bmi-1^{fl/fl} control (FIG. 12A) or Nestin-CreER^{T2}; Bmi-1^{fl/fl} (FIG. 12B) mice 2 weeks after tamoxifen treatment. Tail DNA was used for positive controls and water for negative control. Since Bmi-1 deficient neurospheres were smaller than control neurospheres, PCR reactions were run for an additional 5 cycles in B as compared to A to detect products. In cases where no PCR product was detected (such as lanes 3 and 7) these samples were excluded from calculations of deletion efficiency. In this experiment, all Nestin-CreER^{T2}; Bmi-1^{fl/fl} neurospheres with successful PCR product amplification showed complete excision of the floxed allele. Note that a non-specific product is visible in lane 4. (FIG. 12C) Body mass of Bmi-1^{fl/fl} control (WT) and Nestin-CreER^{T2}; Bmi-1^{fl/fl} (Δ/Δ) mice 6 months after tamoxifen treatment (n=5-8 mice/genotype). (FIG. 12D) Thickness of the molecular and granular layers of cerebellum from Bmi-1^{fl/fl} control and Nestin-CreER^{T2}; Bmi-1^{fl/fl} mice 10-14 months after tamoxifen treatment (n=3-4 mice/genotype with >8 measurements from 2 sections/mouse). (FIGS. 12E-F) The number of Mcm2⁺ (FIG. 12E) and Ki67⁺ (FIG. 12F) SVZ cells per section (n=4-11 mice/genotype/age in 7 independent experiments). (FIG. 12G) Representative images of SVZ sections from 6- and 12-month old Bmi-1^{fl/fl} control (WT) and Nestin-CreER^{T2}; Bmi-1^{fl/fl} (Δ/Δ) mice. (FIG. 12H) RT-PCR was performed on pre-GEPCOT cells isolated from wild type (+/+; 3 months old), Bmi-1^{fl/fl} (fl/fl), or Nestin-CreER^{T2}; BMi-1^{fl/fl} mice (Δ/Δ) 6 months after tamoxifen treatment to assess p16 ^{Ink4a} transcript levels. Amplification of specific products was observed in 2 of 3 Δ/Δ samples but not in any others. Data represent mean ±s.d. Statistical significance was assessed with two-tailed student's t-tests (**p<0.01, *** p <0.001).

### DETAILED DESCRIPTION

Neurosphere formation is commonly used as a surrogate for neural stem cell (NSC) function but the relationship between neurosphere-initiating cells (NICs) and NSCs remains unclear. The inventors now report on the prospective identification of two phenotypically and functionally distinct populations of cells in the SVZ: GEPCOT cells and pre-GEPCOT cells. The pre-GEPCOTs accounted for 6±3% of adult mouse SVZ cells, were highly quiescent, lacked the ability to form neurospheres or adherent colonies in culture, and included type B1 cells based on marker expression, morphology, and position in vivo. These cells contained long-lived qNSCs based on both fate mapping and temozolomide resistance. GEPCOTs were distinguished by lower GFAP and Glast expression and higher EGFR and PlexinB2 expression. These cells accounted for 3.2±0.7% of cells in the adult mouse SVZ, were highly mitotically active, highly enriched for NICs, and included type C cells based on marker expression, morphology, and position in vivo. Based on fate-mapping these cells were short-lived in the SVZ. These data provide methods to prospectively identify and distinguish qNSCs from NICs. These and other aspects of the disclosure are described in greater detail below.

I. Neural Stem Cells and Neurosphere Initiating Cells

A. Neural Stem Cells

Neural stem cells (NSCs) are self-renewing, multipotent cells that generate neurons, astrocytes, and oligodendrocytes. NSCs are hypothesized to maintain themselves for long periods of time in vivo, to regenerate the SVZ after ablation by anti-mitotic agents, and to form differentiated cells through a mix of symmetric and asymmetric divisions. Generally NSCs are quiescent, dividing infrequently in vivo.

B. Neurosphere Initiating Cells

A neurosphere is a free-floating colony of neural progenitor cells. Since neural stem/progenitor cells are difficult to study in vivo, neurospheres have historically provided a method to investigate these cells in vitro. To grow neurospheres, a mixture of neural stem/progenitor cells is added to non-adherent cultures containing necessary growth factors, such as epidermal growth factor and fibrobalst growth factor. The cells are added to culture at very low cell density (<1 cell/μl culture medium). This allows individual neural progenitor cells to form characteristic spherical colonies, such that the neurospheres are clonally derived. Many neurospheres undergo multilineage differentiation to neurons, astrocytes, and oligodendrocytes and exhibit self-renewal potential. That is, upon subcloning, individual single-cell-derived neurospheres commonly give rise to hundreds of multipotent secondary neurospheres.

For decades it has been assumed that neurosphere-initiating cells are the neural stem cells of the SVZ. However, the current disclosure shows that these (GEPCOT) cells are highly mitotically active and short-lived in the SVZ. Neurosphere-initiating cells are ablated by treatment with anti-mitotic agents, such as temozolomide, and are unable to regenerate the SVZ after ablation. In contrast, the current disclosure identifies an earlier quiescent stem cell population (pre-GEPCOT cells) that co-exists with neurosphere-initiating cells in the SVZ. The pre-GEPCOT cells are long-lived in vivo, give rise to large numbers of progeny, survive treatment with anti-mitotic agents, and regenerate the SVZ.

II. Surface Markers

The following surface markers have been identified as relevant to characterizing neural stem cells and neurosphere initiating cells.

A. Glast

GLutamate ASpartate Transporter (GLAST) is a proten that, in humans, is encoded by the gene SLC1A3 (solute carrier family 1a, member 3). GLAST is also often called Excitatory Amino Acid Transporter 1 (EAAT1). GLAST is predominantly expressed in the plasma membrane, allowing it to remove glutamate from the extracellular space. It has also been localized in the inner mitochrondrial membrane as part of the malate-aspartate shuttle.

GLAST functions in vivo as a homotrimer. GLAST mediates the transport of glutamic and aspartic acid with the cotransport of three Na+ and one H+ cations and counter transport of one K+ cation. This co-transport coupling (or symport) allows the transport of glutamate into cells against a concentration gradient.

GLAST is expressed throughout the CNS, and is highly expressed in astrocytes and Bergmann glia in the cerebellum. In the retina, GLAST is expressed in Muller cells. GLAST is also expressed in a number of other tissues including cardiac myocytes.

B. Epidermal Growth Factor Receptor (EGFR)

The epidermal growth factor receptor (EGFR; ErbB-1; HER1 in humans) is the cell-surface receptor for members of the epidermal growth factor family (EGF-family) of extracellular protein ligands. The epidermal growth factor receptor is a member of the ErbB family of receptors, a subfamily of four closely related receptor tyrosine kinases: EGFR (ErbB-1), Her2/c-neu (ErbB-2), Her 3 (ErbB-3) and Her 4 (ErbB-4). Mutations affecting EGFR expression or activity could result in cancer.

EGFR exists on the cell surface and is activated by binding of its specific ligands, including epidermal growth factor and transforming growth factor a (TGFα) (note, a full list of the ligands able to activate EGFR and other members of the ErbB family is given in the ErbB article). ErbB2 has no known direct activating ligand, and may be in an activated state constitutively or become active upon heterodimerization with other family members such as EGFR. Upon activation by its growth factor ligands, EGFR undergoes a transition from an inactive monomeric form to an active homodimer although there is some evidence that preformed inactive dimers may also exist before ligand binding.¹ In addition to forming homodimers after ligand binding, EGFR may pair with another member of the ErbB receptor family, such as ErbB2/Her2/neu, to create an activated heterodimer. There is also evidence to suggest that clusters of activated EGFRs form, although it remains unclear whether this clustering is important for activation itself or occurs subsequent to activation of individual dimers.

EGFR dimerization stimulates its intrinsic intracellular protein-tyrosine kinase activity. As a result, autophosphorylation of several tyrosineresidues in the C-terminal domain of EGFR occurs. These include Y992, Y1045, Y1068, Y1148 and Y1173. This autophosphorylation elicits downstream activation and signaling by several other proteins that associate with the phosphorylated tyrosines through their own phosphotyrosine-binding SH2 domains. These downstream signaling proteins initiate several signal transduction cascades, principally the MAPK, Akt and JNK pathways, leading to DNA synthesis and cell proliferation. Such proteins modulate phenotypes such as cell migration, adhesion, and proliferation. Activation of the receptor is important for the innate immune response in human skin. The kinase domain of EGFR can also cross-phosphorylate tyrosine residues of other receptors it is aggregated with, and can itself be activated in that manner.

C. PlexinB2

PlexinB2 is a protein that in humans is encoded by the PLXNB2 gene. Members of the B class of plexins, such as PLXNB2 are transmembrane receptors that participate in axon guidance and cell migration in response to semaphorins. PLXNB2 has been shown to interact with ARHGEF 11.

D. CD24

Signal transducer CD24 (cluster of differentiation 24) or heat stable antigen (HSA) is encoded by the CD24 gene in humans. CD24 is a cell adhesion molecule. CD24 is a glycoprotein expressed at the surface of most B lymphocytes and differentiating neuroblasts. This gene encodes a sialoglycoprotein that is expressed on mature granulocytes and in many B cells. The encoded protein is anchored via a glycosyl phosphatidylinositol (GPI) link to the cell surface. An alignment of this gene's sequence finds genomic locations with similarity on chromosomes 3p26, 15q21, 15q22, 20q11.2 and Yq11.1. Whether transcription, and corresponding translation, occurs at each of these other genomic locations needs to be experimentally determined (source: NCBI).

E. O4

Oligodendrocyte Marker O4 is an antigen on the surface of oligodendrocyte progenitors. O4 has been commonly used as the earliest recognized marker specific for the oligodendroglial lineage. The monoclonal antibodies A2B5, O4, and O1 are frequently used to define distinct stages in the maturation of oligodendrocyte progenitors. In general, A2B5+/O4− defines the most immature oligodendrocyte precursors, O4+/O1− defines intermediate precursors, and O1+ defines oligodendrocytes at a more mature stage. In many CNS disorders, such as stroke, multiple sclerosis, and spinal cord injury, demyelination of axons contributes to functional deficit.

F. PSA-NCAM

Neural Cell Adhesion Molecule (NCAM, also CD56) is a homophilic binding glycoprotein expressed on the surface of neurons, glia, skeletal muscle and natural killer cells. NCAM has been implicated as having a role in cell-cell adhesion, neurite outgrowth, synaptic plasticity, and learning and memory.

NCAM is a glycoprotein of Immunoglobulin (Ig) superfamily. At least 27 alternatively spliced NCAM mRNAs are produced, giving a wide diversity of NCAM isoforms. The three main isoforms of NCAM vary only in their cytoplasmic domain:

NCAM-120 kDa (GPI anchored)

NCAM-140 kDa (short cytoplasmic domain)

NCAM-180 kDa (long cytoplasmic domain)

The extracellular domain of NCAM consists of five immunoglobluin-like (Ig) domains followed by two fibronectin type III (FNIII) domains. The different domains of NCAM have been shown to have different roles, with the Ig domains being involved in homophilic binding to NCAM, and the FNIII domains being involved signaling leading to neurite outgrowth.

Homophilic binding occurs between NCAM molecules on opposing surfaces (trans-) and NCAM molecules on the same surface (cis-)1. There is much controversy as to how exactly NCAM homophilic binding is arranged both in trans- and cis-. Current models suggest trans-homophilic binding occurs between two NCAM molecules binding antiparallel between all five Ig domains or just IgI and IgII. cis-homophilic binding is thought to occur by interactions between both IgI and IgII, and IgI and IgIII, forming a higher order NCAM multimer. Both cis- and trans-NCAM homophilic binding have been shown to be important in NCAM “activation” leading to neurite outgrowth.

NCAM exhibits glycoforms as it can be post-translationally modified by the addition of polysialic acid (PSA) to the fifth Ig domain, which is thought to abrogate its homophilic binding properties and can lead to reduced cell adhesion important in cell migration and invasion. PSA has been shown to be critical in learning and memory. Removal of PSA from NCAM by the enzyme endomeuraminidase (EndoN) has been shown to abolish long-term potentiation (LTP) and long-term depression (LTD).

G. Ter119

The monoclonal antibody of this name recognizes an epitope on murine erythroid cells. The TER-119 antigen is a moiety associated with cell-surface glycophorin A, but not identical with glycophorin A. An alternative designation is Ly76.

TER-119 is used as a lineage marker for erythroid cells. TER-119 has been reported to react with mature erythrocytes, 20-25% of bone marrow cells, and 2-3% of spleen cells, but not with thymocytes or lymph node cells. In fetal hematopoietic tissues, 30-40% of day 10 yolk sac cells, 80-90% of day 14 fetal liver cells, and 40-50% of newborn liver cells are TER-119(+). TER-119(+) cells in adult bone marrow express significant levels of CD45 but not myeloid markers (Mac-1, Gr-1) or B-cell markers (B220). The antibody reacts with erythroid cells at different stages of development (early pro-erythroblasts to mature erythrocytes). It does not react with more developed cells such as BFU-E or CFU-E. Erythroleukemia cell lines with or without stimulation are TER-119(−). TER-119 and other markers can be used to deplete cell populations of mature blood cells in order to obtain enriched populations of hematopoietic stem cells.

H. CD45

CD45 antigen, which was originally called leukocyte common antigen (LCA), is a protein tyrosine phosphatase, receptor type, C. It is also known as PTPRC. CD45 is a member of the protein tyrosine phosphatase (PTP) family. PTPs are known to be signaling molecules that regulate a variety of cellular processes including cell growth, differentiation, mitotic cycle, and oncogenic transformation. This PTP contains an extracellular domain, a single transmembrane segment and two tandem intracytoplasmic catalytic domains, and thus belongs to receptor type PTP. This gene is specifically expressed in hematopoietic cells. This PTP has been shown to be an essential regulator of T- and B-cell antigen receptor signaling. It functions through either direct interaction with components of the antigen receptor complexes or by activating various Src family kinases required for the antigen receptor signaling. This PTP also suppresses JAK kinases, and, thus, functions as a negative regulator of cytokine receptor signaling. Four alternatively spliced transcripts variants of this gene, which encode distinct isoforms, have been reported.

CD45 is a type I transmembrane protein that is in various forms present on all differentiated hematopoietic cells cells except erythrocytes and plasma cells that assists in the activation of those cells (a form of co-stimulation). It is expressed in lymphomas, B-cell chronic lymphocytic leukemia, hairy cell leukemia, and acute non-lymphocytic leukemia. A monoclonal antibody to CD45 is used in routine immunohistochemistry to differentiate between lymphomas and carcinomas on histological sections.

III. Methods of Isolating Cells

A. General Methodologies

Fluorescence-Cytometry. A useful approach to identifying and isolating cells according to surface markers is flow cytometry, a specialized type of flow cytometry. It provides a method for sorting a heterogeneous mixture of biological cells into two or more containers, one cell at a time, based upon the specific light scattering and fluorescent characteristics of each cell. It provides fast, objective and quantitative recording of fluorescent signals from individual cells as well as physical separation of cells of particular interest. A cell suspension is entrained in the center of a narrow, rapidly flowing stream of liquid. The flow is arranged so that there is a large separation between cells relative to their diameter. A vibrating mechanism causes the stream of cells to break into individual droplets. The system is adjusted so that there is a low probability of more than one cell per droplet. Just before the stream breaks into droplets, the flow passes through a fluorescence measuring station where the fluorescent character of interest of each cell is measured. An electrical charging ring is placed just at the point where the stream breaks into droplets. A charge is placed on the ring based on the immediately prior fluorescence intensity measurement, and the opposite charge is trapped on the droplet as it breaks from the stream. The charged droplets then fall through an electrostatic deflection system that diverts droplets into containers based upon their charge. In some systems, the charge is applied directly to the stream, and the droplet breaking off retains charge of the same sign as the stream. The stream is then returned to neutral after the droplet breaks off One common way to use flow cytometry is with a fluorescently labeled antibody that binds to a target on or in a cell, thereby identifying cells with a given target. This technique can be used quantitatively where the amount of fluorescence corrleates to the amount of target, thereby permitting one to sort based on relative amounts of fluorescence, and hence relative amounts of the target.

B. Technical Considerations When Sorting Adult SVZ Cells For Neurosphere Assays

Neurosphere assays were done as previously published by our lab (Molofsky et al., 2003; Molofsky et al., 2005; Nishino et al., 2008). However, when sorting adult SVZ cells for neurosphere assays several important technical points require attention.

Debris elimination. The extensive debris in adult SVZ cell preps complicates the determination of which events are real cells. This is not a problem for embryonic or neonatal cell preparations. The inventors compared multiple strategies to eliminate debris from adult SVZ preparations. The simplest, least expensive, and most reproducible solution was stringent forward scatter and side scatter gating as shown in FIG. 9A. To use this technique it is important to calibrate the forward scatter and side scatter voltages using freshly isolated bone marrow cells because even a small change in PMT voltages can dramatically affect cell yield and the amount of debris contaminating the cellular fraction. Before every experiment the inventorsaligned the myeloid population into the center of the forward scatter/side scatter plot [FSC-Area =125k, SSC-Area =125k] then the inventorsensured that the forward scatter threshold was set as high as possible without eliminating cells (typically 75000 and did not require daily adjustment). When the forward scatter and side scatter voltages are properly calibrated there should be a prominent population of adult SVZ cells with similar FSC/SSC as lymphoid cells.

There are drawbacks to stringent forward scatter and side scatter gating. Some debris has similar FSC/SSC characteristics as live SVZ cells (typically 10% of events in the live SVZ scatter fraction represent debris as judged by DAPI staining after sorting on slides), and some cells are lost in the debris exclusion gate including a few cells with neurosphere-forming activity (generally 25% of NICs are excluded by the debris exclusion gate) and all ependymal cells (which have very high side scatter properties due to their cilia). As a result the inventors also explored other options to increase cell purity and yield which they summarize here.

The inventors tried two other techniques to eliminate debris: sucrose gradient and myelin depletion. For sucrose gradient, the inventorsresuspended pelleted SVZ cells in 10 ml of 0.9M sucrose in staining medium, then centrifuged the cells (750 g, 10 min, 4° C.) and decanted the supernatant. The sucrose gradient typically eliminated ˜90% of debris but also lost ˜50% of cells. The inventors performed myelin depletion using the Myelin Removal Beads II kit (Miltenyi Biotec, 130-096-733) according to the manufacturer's instructions. Myelin depletion typically eliminated ˜95-98% of debris but also eliminated ˜50% of cells. Because of the unacceptable losses in cell yields, for routine experiments the inventors did not eliminate debris using either of these methods. However, when cell purity is paramount these debris elimination techniques may be useful.

The inventors also attempted other techniques to enrich for cells and to deplete debris using vital dyes. The inventors tried Calcein AM (Life Technologies, C3099) and CFSE (Life Technologies, C1157) but these stains did not distinguish adequately between cells and debris. The inventors also tried Hoechst 33342 (Life Technologies, H1399) and Vybrant DyeCycle Violet (Life Technologies, V35003), and although these stains did distinguish between cells and debris the inventors found that these reagents were not useful because 1) the intensity of staining varied substantially across days, 2) staining of other markers (in particular Glast) was negatively affected by using these reagents, and 3) efficiency of clonogenesis after sorting was negatively impacted by using either of these reagents. As a result, the inventors did not use these dyes for routine experiments, but they could be useful when cell yield is paramount.

Finally, the inventors also used the fluorescent transgenic lines Nestin-GFP and Nestin-mCherry (Birbrair et al., 2011; Ding et al., 2012). These lines allowed easy discrimination of SVZ cells from debris because of their bright, unambiguous expression in cells. Using Nestin-GFP or Nestin-mCherry and gating on GFP⁺DAPI⁻ (or mCherry⁺ DAPI⁻) events typically gave ˜50% greater cell yield and greater cell purity than when only using scatter-gating for cell identification. This is presumably because some Nestin-GFP+ or Nestin-Cherry+ cells (such as ependymal cells) appear as debris by FSC/SSC profile and would otherwise be excluded. However, the inventors did not wish to use transgenic lines to purify GEPCOT or pre-GEPCOT cells as this would limit the technique to these genetic backgrounds, and because express these transgenes are not uniformly expressed by pre-GEPCOT cells. However, for certain applications where high cell purity and yield are both paramount, or ependymal cells are desired, then Nestin-GFP or Nestin-mCherry may be useful.

EDTA in staining medium increased GEPCOT cell yield. In the inventors' experiments, the staining medium the inventors employed contained 3.0 mM EDTA, enough to neutralize 2.7 mM of divalent cations in the staining medium (with a small surplus). The inventors found that this slight excess of EDTA had a large effect (2-3-fold) on the yield of GEPCOT cells, probably by preventing clumping of GEPCOT cells with debris and/or other cells. The EDTA also prevented clogging of the nozzle during sorting. EDTA was found to be better than EGTA for this purpose. However, it was important to maintain the osmolarity of the staining medium when adding EDTA or clonogenesis was negatively impacted. The inventors pre-diluted 0.5M EDTA (made from Na₂H₂EDTA, pH 8.0) to 77.7 mM (309 mOsm) before adding it to the staining medium.

Growth medium pH must be kept neutral during sorting by preventing equilibration with the atmosphere. When sorting cells directly into culture medium it is essential to keep the pH of the medium constant. The phenol red indicator should maintain a red-orange color (pH 7.2-7.4) before, during, and after the sort. Typically, the inventors keep each plate in a Ziploc bag filled with 6% CO₂/93% N₂/1% O₂ prior to the sort, then sort one plate in 2-3 minutes or less, and return it to a freshly gassed Ziploc bag until transferring the plate to a humidified 37° C., CO₂ incubator for culture. By keeping plates in 6% CO₂ before and after the sort, the culture medium does not equilibrate with the air (which would deplete CO₂ from the medium and increase pH to levels incompatible with cell survival).

Rock inhibitor (Y-27632) and IGF1 increase clonogenesis after sorting. The process of being sorted has a negative effect on neurosphere formation from adult SVZ cells: at the beginning of their experiments the inventors observed a 3-fold loss of activity in sorted live SVZ cells compared to crude unsorted cells. Sorted neonatal SVZ cells do not show this problem. The inventors wondered how to sort gently to overcome the fragility of adult SVZ cells.

Addition of the ROCK inhibitor, Y-27632 hydrochloride, to the culture medium after sorting (10 μM) increased neurosphere formation by adult cells approximately two-fold after sorting but did not have any effect on adult SVZ cells pipetted into the culture medium, on sorted neonatal SVZ cells, or on crude neonatal SVZ cells. The inventors also found that IGF1 (20 ng/ml) further improved clonogenesis after sorting. The mechanisms underlying these effects are unclear but may involve promotion of cell survival after sorting or repression of BMP signaling (Hsieh et al., 2004). When using both these culture additives, there was little difference between sorted live SVZ cells and an equal number of cells added to culture by pipette in terms of the numbers of neurospheres they made.

Preventing neurosphere aggregation after sorting. Neurospheres can aggregate and fuse in culture, particularly when cultured at higher densities or when plates are moved (facilitating collisions among neurospheres) during the culture period (Singec et al., 2006). To minimize this problem, the inventors took several precautions. First, the inventors always sorted cells into culture at clonal (low) density: 1000 cells in 1.5 ml medium (0.66 cells/μ1) in each well of a 6-well plate. Second, the inventors maintained a separate incubator specifically for neurosphere cultures that was only opened sparingly to prevent mechanical disturbances or changes in CO₂ concentrations.

Finally, the inventors found that chick embryonic extract (CEE) (Molofsky et al., 2003) can prevent cell fusion after sorting. In the absence of CEE, sorted cell-derived primary neurospheres were few in number and very large, suggesting extensive sphere fusion. After adding 10% CEE to the culture medium, sorted cell-derived primary neurospheres were indistinguishable in size and number from neurospheres cultured from cells pipetted into culture. The inventors found that the combination of 10% CEE plus 10 μM Y-27632 hydrochloride plus 20 ng/mL IGF1 maximized the formation of non-aggregating neurospheres from sorted adult SVZ cells.

TABLE 1
Antibodies Reacting Positively in Marker Screen for Neurosphere Initiating
Cells

| | | | Enriches | |
|---|---|---|---|---|
| | Antibody | | neurosphere- | |
| Antigen | source | All SVZ cells? | initiating cells? | GEPCOT cells? |
| CD45 cl. 30-F11 | Biolegend | Heterogeneous | Yes | — |
| Ter119 | Biolegend | Heterogeneous | Yes | — |
| CD24 cl. M1/69 | eBioscience | Heterogeneous | Yes | —/low |
| O4 | Morrison lab | Heterogeneous | Yes | —/low |
| PSA-NCAM cl. | DSHB | Heterogeneous | Yes | —/low |
| 5A5 | | | | |
| CD200 cl. OX-90 | BD | Heterogeneous | Yes | Heterogeneous but neurosphere- |
| | Bioscience | | | initiating cells are enriched in |
| | | | | —/low fraction |
| 8.1.1 | DSB | Heterogeneous | Yes | Heterogeneous but neurosphere- |
| | | | | initiating cells are enriched in high |
| | | | | fraction |
| CD172a cl. P84 | BD | Heterogeneous | Yes | Heterogeneous but neurosphere- |
| | Bioscience | | | initiating cells are enriched in |
| | | | | —/low fraction |
| EGFR cat. | R&D | Heterogeneous | Yes | High |
| BAF1280 | Systems | | | |
| PlexinB2 cl. 3E7 | eBioscience | Heterogeneous | Yes | High |
| Glast cl. ACSA-1 | Miltenyl | Heterogeneous | Yes | Mid |
| HNK1/CD57 cl. | Sigma | Heterogeneous | Yes | —/low |
| VC1.1 | | | | |
| CD81 cl. Eat-2 | eBioscience | Heterogeneous | Yes | High |
| CD56 cl. 13 | BD | Heterogeneous | Yes | —/low |
| | Bioscience | | | |
| LTA lectin | EY Labs | Heterogeneous | Yes | —/low |
| PHA-L lectin | EY Labs | Heterogeneous | Yes | —/low |
| 647-Egf | Life | Heterogeneous | Yes | High |
| | Technologies | | | |

IV. Kits

For use in the applications described herein, kits are also within the scope of the disclosure. Such kits can comprise a carrier, package or container that is compartmentalized to receive one or more containers such as vials, tubes, and the like, each of the container(s) comprising one of the separate elements to be used in the method, in particular, one or more antibodies that bind to a marker defining a neurosphere initiating cell and/or a neural stem cell. The kit of the disclosure will typically comprise the container described above and one or more other containers comprising materials desirable from a commercial end user standpoint, including buffers, diluents, filters, tubes, needles, plates, syringes, and package inserts with instructions for use. In addition, a label can be provided on the container to indicate that the composition is used for a specific application, and can also indicate directions for use, such as those described herein. Directions and or other information can also be included on an insert which is included with the kit. In particular, kits according to the present disclosure contemplate the assemblage of agents for assessing surface protein marker expression along with one or more agents for performing fluorescent activated cell sorting, as well as controls for assessing the same.

### EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Materials and Methods

Mice. C57B1/6 mice were maintained in standard cages with water and standard diet (Teklad 2916) ad libitum. Rosa26^{CAG-loxp-Stop-loxp-tdTomato(Ai14)} (referred to here as loxp-tdTomato) (Madisen et al., 2010), Gli1^{CreERT2} (Ahn and Joyner, 2005), Sox2^{CreERT2} (Arnold et al., 2011), Dlx1^{CreERT2} (Taniguchi et al., 2011), Tg(Glast-CreER^{T}) (Wang et al., 2012), Tg(Nestin-Cre) (Tronche et al., 1999) and Tg(Ubc-GFP) (Schaefer et al., 2001) mice were obtained from The Jackson Laboratory. Tg(GFAP-CreER^{T2}) (Hirrlinger et al., 2006) mice were provided by Frank Kirchhoff. Tg(Nestin-GFP) (Birbrair et al., 2011) and Tg(Nestin-mCherry) (Ding et al., 2012) mice were kindly provided by Grigori Enikopalov. Tg(Nestin-CreER^{T2}) mice were provided by G. Fishell (Balordi and Fishell, 2007). All mice were backcrossed onto a C57BL/Ka background for at least 3 generations prior to analysis. For BrdU pulses up to 24 hours, 100 mg of BrdU/kg body mass dissolved in PBS was injected i.p. every six hours. For 2-week pulses of BrdU, mice were initially injected with 100 mg of BrdU/kg body mass then maintained on drinking water that contained 1 mg/ml BrdU until sacrifice.

Targeting vectors to generate Bmi1^{fl} mice (FIG. 10A) were constructed by recombineering (Liu et al., 2003). W4 ES cells were electroporated with the targeting vector and positive clones identified by southern blotting were injected into blastocysts from C57BL/6-Tyrc-2J mice. The resulting male chimeric mice were bred to female C57BL/6-tyrc-2J mice to obtain germline transmission. The FRT-Neo-FRT cassette was removed by crossing with Flp deleter mice (Rodriguez et al., 2000), and then Bmi1^{fl} mice were backcrossed onto a C57BL/Ka background for at least 10 generations. Animal protocols were approved by the University of Michigan Committee on the Use and Care of Animals and the UT Southwestern Medical Center Institutional Animal Care and Use Committee (protocol# 2011-0104).

Induction of recombination using tamoxifen. Tamoxifen (Sigma T5648) was dissolved in 90% corn oil/10% ethanol at 20 mg/ml, and injected at 80 mg/kg/day i.p. for 5 consecutive days into 8-12 week-old mice, then the mice were chased for 2, 7, 28, or 60 days until analysis. For Bmi-1 experiments, tamoxifen citrate (Sigma or Spectrum Chemical) was given in chow at 400 mg/kg with 5% sucrose (Harlan) to 6 week-old mice for 30 days. Mice were then fed a standard diet for at least 14 days before analysis.

TMZ regeneration assay. TMZ (temozolomide; Sigma T2577) was dissolved in 25% DMSO/75% 0.9% saline solution at 10 mg/ml by heating briefly to 90-100° C., then shaking and rapidly cooling. Mice were injected at 100 mg/kg/day for three consecutive days then allowed to recover for 3-90 days before analysis. Mice with enlarged spleen or thymus (sometimes present at 90 days after TMZ treatment) were excluded from analysis.

SVZ cell preparation. SVZs from adult (8-60 week old) mice were dissected as described (Mirzadeh et al., 2010). SVZs were minced and digested with 300 μl of trypsin solution (Ca and Mg-free HBSS, 10 mM HEPES, 0.5 mM EDTA, 0.25 mg/mL trypsin (EMD Millipore), 10 μg/mL DNase I (Roche), pH 7.6) at 37° C. for 20 minutes. Digestion was quenched with three volumes of staining medium (440 ml Leibovitz L-15 medium, 50 ml water, 5 ml 1M HEPES pH 7.3-7.4, 5 ml 100× Pen-Strep, 20 ml 77.7 mM EDTA pH 8.0 [prepared from Na2H2EDTA], 1 g bovine serum albumin [Sigma A7030]) containing 100 μg/ml trypsin inhibitor (Sigma T6522) and 10 μg/ml DNase I (Roche).

Digested SVZ pieces were centrifuged (220 g, 4 min, 4° C.) then fresh staining medium was added and the pieces were triturated in 300 μl by gently drawing into a P1000 pipetman and expelling 25 times without forming bubbles. The cell suspension was then filtered through a 45-micron mesh, counted on a hemocytometer, and added to culture or processed for flow cytometry. Neurosphere formation, self-renewal, and differentiation assays were performed as described previously (Molofsky et al., 2003; Nishino et al., 2008), except that in some cases 10 μM Y-27632 hydrochloride (Rho-associated protein kinase inhibitor; Tocris Biosciences 1254) and 20 ng/ml IGF-1 (R&D Systems 291-G1) were also added to the culture medium because they were found to promote clonogenesis after sorting adult SVZ cells (data not shown). For adherent colony formation, Tg(Ubc-GFP) SVZ cells were co-cultured with 80-90% confluent nontransgenic neonatal SVZ-derived astrocytes isolated and passaged twice prior to the experiment. GFP fluorescence was used to assess colony formation after 12-20 days in culture.

Flow cytometry and sorting. For screening for markers of NICs, typically SVZs from 5-10 mice were pooled and stained with antibodies then analyzed using a 4 laser FACSAria III (Becton Dickenson). Markers that stained SVZ cells heterogeneously were used to separate SVZ cells by flow cytometry into different fractions then sorted into non-adherent cultures to measure the frequency of NICs.

To stain SVZ cells with the combination of markers used to isolate GEPCOT or pre-GEPCOT cells, dissociated SVZ cells were centrifuged (220 g, 4 min, 4° C.) and resuspended in 100 μl staining medium per brain. Then the following antibodies were added: BV421 anti-Ter119 (Biolegend 116233, 1/100), BV421 anti-CD45 (Biolegend 103133, 1/100), APC-eFluor 780 anti-CD24 (eBioscience 47-0242-82, 1/200), 5A5 ascites (anti-PSA-NCAM; Developmental Studies Hybridoma Bank, 1/100), O4 ascites (1/200), anti-PlexinB2 (eBioscience 14-5665-82, 1/100), biotinylated anti-EGFR (R&D Systems BAF1280, 1/200), anti-Glast (Miltenyi Biotec 130-095-822, 1/10), and DAPI (50 μg/ml, 1/100). After adding antibodies, the cells were incubated on ice for 45 minutes, then washed with 2.5 ml fresh staining medium and pelleted (220 g for 4 minutes at 4° C.) and resuspended in secondary antibodies. Typically, the inventors used the following combination: APC anti-mouse IgG2a (Jackson Immunoresearch 115-135-206,1/200), PE anti-hamster IgG (Jackson Immunoresearch 127-115-160, 1/200), PE-Cy7 anti-mouse IgM (eBioscience 25-5790-82, 1/100), BV605-Streptavidin (BD Biosciences 563260, 1/200). However, when analyzing mice expressing tdTomato, the inventors used the following alternative secondary antibodies: PerCP-eFluor 710 anti-mouse IgG (eBioscience 46-4010-82, 1/100), APC anti-hamster IgG (Jackson Immunoresearch 127-135-160, 1/200), PE-Cy7 anti-mouse IgM (eBioscience 25-5790-82, 1/100), and BV605-Streptavidin (BD Biosciences 563260, 1/200). Cells were stained with secondary antibodies on ice for 45 minutes, then washed and pelleted as above, and analyzed on the FACSAria in a volume of 200 μl per brain.

When sorting cells onto slides, a drop of 8 μl staining medium was placed on a SuperFrost Plus slide, and 100-300 cells were sorted directly into the drop. The slide was then kept humid for 45-60 minutes at ambient temperature to allow the cells to attach to the glass, and then the cells were fixed with 4% PFA (20 minutes, RT). Cells were then permeabilized and stained with rat anti-BrdU (Abcam clone Bu1/75, 1/500, after heat-mediated antigen retrieval), mouse anti-Nestin (BD clone Rat-401, 1/100 after heat-mediated antigen retrieval), and rabbit anti-GFAP (Dako, 1/3000) antibodies using standard techniques. Immunostaining. Brains were fixed overnight at 4° C. in 4% PFA in PBS, then cryoprotected in 30% sucrose in PBS for 1-3 days at 4° C., then frozen in OCT or cryogel on dry ice after 3-12 hours equilibration at 4° C. Sections were cut at 12 μm thickness spanning the rostral half of the SVZ (typically 6 sections per slide) or the entire olfactory bulb (typically 8 sections per slide). Sections were immunostained with the following primary antibodies: rat anti-BrdU (Abeam clone Bu1/75, 1/500, after heat-mediated antigen retrieval), guinea pig anti-Dcx (Millipore 1/1000), mouse anti-Mcm2 (BD Biosciences, ¹/₅00, after heat-mediated antigen retrieval), rat anti-Ki67 (eBioscience, 1/500), mouse anti-tyrosine hydroxylase (Millipore, 1/1000), rabbit anti-calretinin (Sigma, 1/1000), rabbit anti-calbindin (Millipore, 1/500), rabbit anti-S100(3 (Dako, 1/1000), rabbit anti-GST-pi (Enzo, 1/3000), and mouse anti-NeuN (Millipore, 1/1000). Fixed whole-mount SVZs were stained with mouse anti-acetylated tubulin (Sigma, 1/1000), rabbit anti-β-catenin (Sigma, 1/500), mouse anti-GFAP (Sigma, 1/3000), and goat anti-EGFR (R&D Systems, 1/250). Alexa Fluor 488-, 555-, and 647-conjugated secondary antibodies were used (Life Technologies).

Western blotting. Cells were resuspended in 10% trichloracetic acid (TCA, Sigma). Extracts were incubated on ice for at least 15 minutes and centrifuged at 16,100×g at 4° C. for 15 minutes. Precipitates were washed in acetone twice and dried. The pellets were solubilized in 9M urea, 2% Triton X-100, and 1% DTT. LDS loading buffer (Invitrogen) was added and the pellet was heated at 70° C. for 10 minutes. Samples were separated on Bis-Trispolyacrylamide gels (Invitrogen) and transferred to PVDF membrane (Millipore or BioRad). Membranes were treated with the SuperSignal Western Blot Enhancer (Thermo Scientific) and blots were developed with the SuperSignal West Femto chemiluminescence kit (Thermo Scientific). Blots were stripped with 1% SDS, 25 mM glycine (pH 2) prior to reprobing. The following primary antibodies were used for western blots: β-Actin (AC-15; Santa Cruz), Bmi-1 (F6; Millipore).

Quantitative PCR. Cells were sorted directly into RLT plus buffer (Qiagen) supplemented with 2-mercaptoethanol. RNA was extracted with the RNeasy micro plus kit (Qiagen) and cDNA was synthesized with the RT2 First Strand Kit (Qiagen). Reactions were run in 20 μl volumes with SYBR green and a LightCycler 480 (Roche Applied Science). Primer sequences were:

(SEQ ID NO: 1)

| | |
|---|---|
| Bmi-1 F (FIG. 6A), | 5′-CCAATGGCTCCAATGAAGACC-3′, |

(SEQ ID NO: 2)

| | |
|---|---|
| Bmi-1 R (FIG. 6A), | 5′-TTGCTGCTGGGCATCGTAAG-3′, |

(SEQ ID NO: 3)

| | |
|---|---|
| Bmi-1 F (FIG. 10G), | 5′-CGCTCTTTCCGGGATCTTT-3′, |

(SEQ ID NO: 4)

| | |
|---|---|
| Bmi-1 R (FIG. 10G), | 5′-CTCCACACAGGACACACATTA-3′, |

(SEQ ID NO: 5)

| | |
|---|---|
| p16^{Ink4α} F, | 5′-GTGTGCATGACGTGCGGG-3′, |

(SEQ ID NO: 6)

| | |
|---|---|
| p16^{Ink4α} R, | 5′-GCAGTTCGAATCTGCACCGTAG-3′; |

(SEQ ID NO: 7)

| | |
|---|---|
| β-actin F, | 5′-CGTCGACAACGGCTCCGGCATG-3′; |

(SEQ ID NO: 8)

| | |
|---|---|
| β-actin R, | 5′-GGGCCTCGTCACCCACATAGGAG-3′; |

(SEQ ID NO: 9)

| | |
|---|---|
| Nestin F | 5′-GGGCCCAGAGCTTTCCCACG-3′; |

(SEQ ID NO: 10)

| | |
|---|---|
| Nestin R | 5′-GGGCATGCACCAGACCCTGTG-3′. |

### Example 2

### Results

Prospective identification of NICs. The inventors enzymatically dissociated adult mouse SVZ cells then sorted cells by flow cytometry into non-adherent cultures at clonal density (0.66 cells/μl of culture medium). The inventors always replated neurospheres to adherent secondary cultures to assess differentiation into TuJ1⁺ neurons, GFAP⁺ astrocytes, and O4⁺ oligodendrocytes. On average, 1.8±0.4% of SVZ cells formed neurospheres (>50 μm diameter) and 75% of those neurospheres underwent multilineage differentiation (1.4±0.3% of SVZ cells).

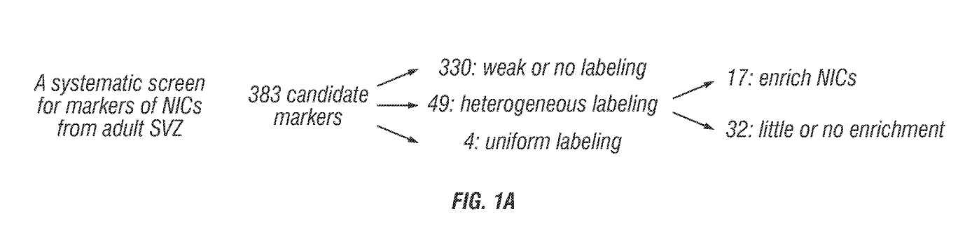

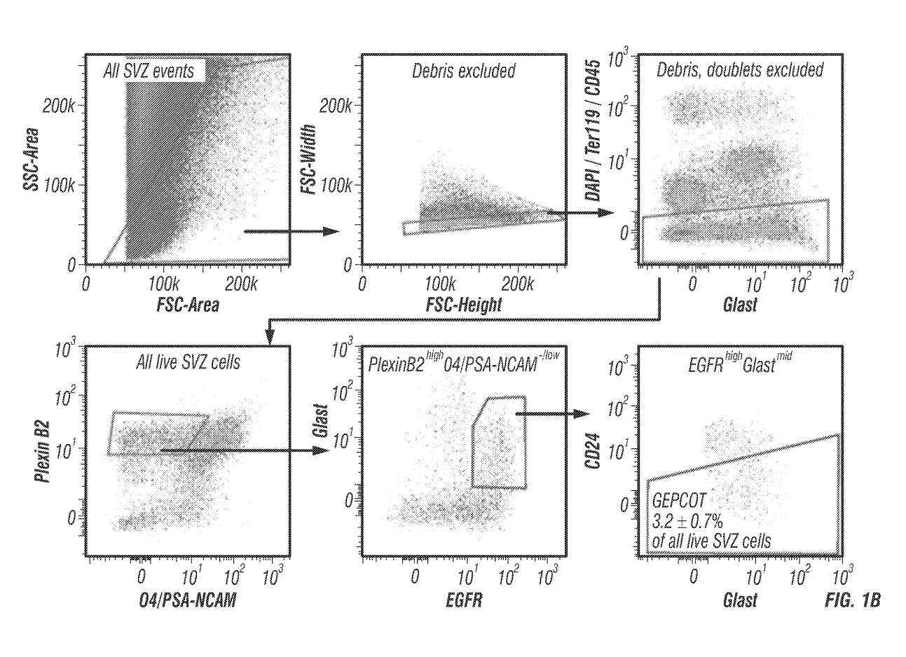

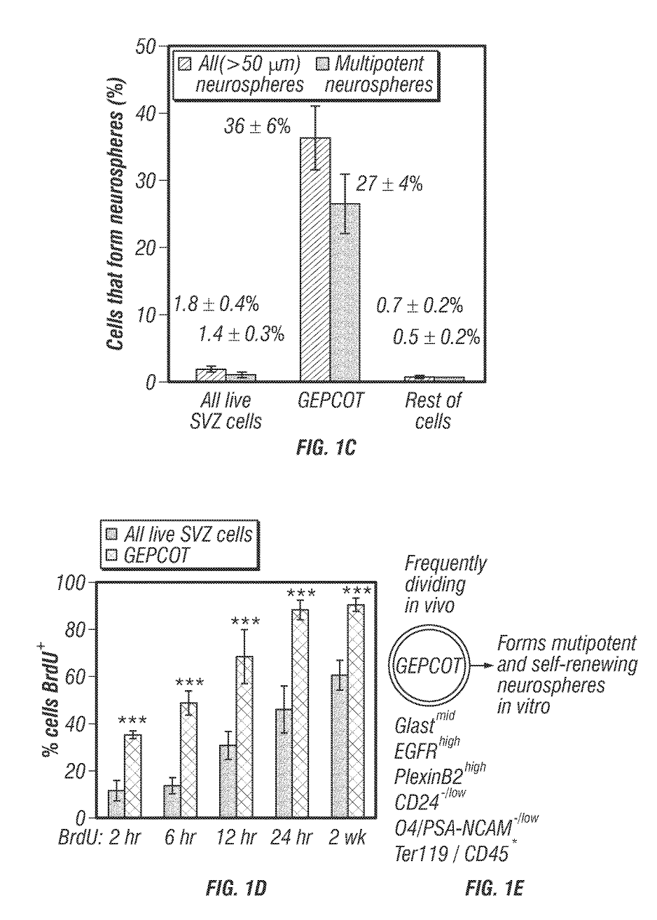

The inventors systematically screened 383 antibodies against 330 distinct cell surface antigens (data not shown) to identify markers that could enrich NICs (FIG. 1A). The inventors identified 49 markers by flow cytometry that were heterogeneously expressed among dissociated SVZ cells. For each of these markers they sorted SVZ cells that differed in their level of staining into non-adherent cultures and assessed neurosphere formation. The inventors found 17 markers that enriched NICs relative to unfractionated SVZ cells (Table 1). The inventors multiplexed combinations of these markers to optimize enrichment while ensuring that most NICs were retained within the sorted population.

The inventors greatly enriched NICs by isolating live (4′,6-diamidino-2-phenylindole (DAPI) negative) SVZ cells that expressed moderate levels of Glast, high levels of Epidermal Growth Factor Receptor (EGFR), high levels of PlexinB2, negative to low levels of CD24, negative to low levels of O4 and PSA-NCAM, and were negative for the hematopoietic markers Ter119 and CD45. The inventors refer to these Glast^{mid}EGFR^{high}PlexinB2^{high}CD24^{-/low}O4/PSA-NCAM^{-/low}Ter119/CD45⁻ cells as GEPCOT cells (FIG. 1B and FIGS. 9A-B). GEPCOTs accounted for 3.2±0.7% of all SVZ cells (FIG. 1B). On average, 36±6% of GEPCOT cells formed neurospheres (>50 μm diameter) and 74% of those neurospheres underwent multilineage differentiation (FIG. 1C). Nearly all of the neurospheres (91%) could be passaged (data not shown). On average, each neurosphere gave rise to 53±41 multipotent secondary neurospheres upon dissociation and replating, demonstrating self-renewal potential. Most NICs from the SVZ were contained within this GEPCOT population (FIG. 9C). Given that individual NICs are unlikely to form colonies with 100% efficiency after dissociation and flow cytometry, most GEPCOT cells likely have the potential to form neurospheres.

NICs are highly proliferative and short-lived in vivo. The ability to prospectively identify NICs made it possible to assess their cell cycle distribution in vivo by administering bromodeoxyuridine (BrdU) to mice. After just a two hour pulse of BrdU, 35±2% of GEPCOTs were already BrdU as compared to only 11±4% of unfractionated SVZ cells (FIG. 1D). Longer pulses of BrdU progressively increased the labeling of the GEPCOT cells. After a 24-hour pulse of BrdU, 89±4% of GEPCOTs were BrdU as compared to 47±9% of unfractionated SVZ cells (FIG. 1D). GEPCOTs are thus highly mitotically active and enriched for dividing cells relative to unfractionated SVZ cells. The observation that nearly all GEPCOTs incorporated BrdU within 24 hours indicates that few, if any, GEPCOTs are quiescent and that NICs are highly proliferative in vivo.

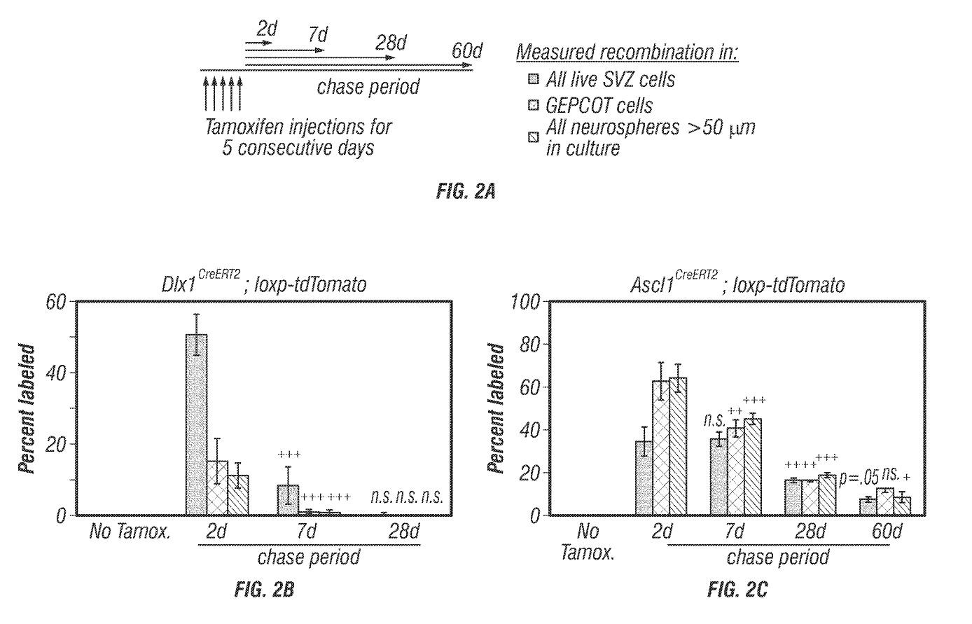

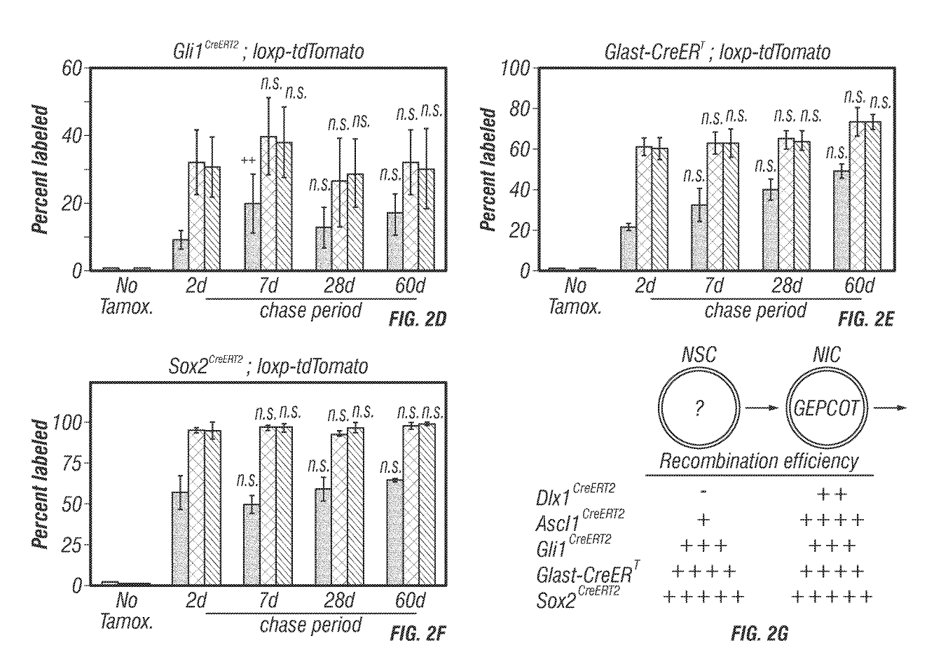

To assess the persistence of NICs in vivo, the inventors performed a series of lineage tracing experiments. Guided by previous observations (Doetsch et al., 2002; Ahn and Joyner, 2005; Arnold et al., 2011; Kim et al., 2011; Taniguchi et al., 2011; Lee et al., 2012; Wang et al., 2012), the inventors screened candidate CreER^{T2} alleles for the ability to recombine a conditional loxp-tdTomato reporter within NICs in vivo after five consecutive days of tamoxifen injection (FIG. 2A; 80 mg/kg body mass/day i.p.). The inventors found that Dlx1^{CreERT2} (Taniguchi et al., 2011), Ascl1^{CreERT2} (Kim et al., 2011), Gli1^{CreERT2} (Ahn and Joyner, 2005), Glast-CreER^{T} (Wang et al., 2012), and Sox2^{CreERT2} (Arnold et al., 2011) labeled 11-94% of NICs two days after tamoxifen administration (FIGS. 2B-F). With each Cre allele and at each time point, the frequency of tdTomato^{T} GEPCOTs was statistically indistinguishable from the frequency of tdTomato^{T} neurospheres that arose in culture from unfractionated SVZ cells (FIGS. 2B-F). This independently confirms that GEPCOT markers reliably identify uncultured cells with the ability to form neurospheres.

To assess the persistence of NICs in vivo, the inventors quantified the frequencies of all SVZ cells, GEPCOTs, and neurospheres that were tdTomato at 2, 7, 28, or 60 days after tamoxifen treatment. In the absence of tamoxifen, the inventors detected no tdTomato expression in SVZ cells, GEPCOTs, or cultured neurospheres in Dlx1^{CreERT2} mice (FIG. 2B). Two days after tamoxifen treatment, Dlx1^{CreERT2} labeled 51±6% of SVZ cells, 15±6% of GEPCOTs, and 11±4% of neurospheres (FIG. 2B). Seven days after tamoxifen this significantly (p<0.001) declined to 8±5% of SVZ cells, 0.7±0.9% of GEPCOTs, and 0.9±1% of neurospheres (FIG. 2B). Twenty-eight days after tamoxifen only rare SVZ cells, GEPCOTs, and neurospheres were labeled (FIG. 2B). Dlx1^{CreERT2}-expressing NICs thus persist in vivo for less than 7 days.

Ascl1^{CreERT2} labeled most GEPCOTs and neurospheres. In the absence of tamoxifen, only 0.02% of SVZ cells, and no GEPCOTs or neurospheres, were tdTomato (FIG. 2C). Two days after tamoxifen, Ascl1^{CreERT2} labeled 34±7% of SVZ cells, 63±8% of GEPCOTs, and 64±6% of neurospheres (FIG. 2C). Seven days after tamoxifen a similar fraction of SVZ cells remained tdTomato (35±3%) but the fraction of labeled GEPCOTs and neurospheres significantly (p<0.01) declined to 40±4% and 45±3%, respectively (FIG. 2C). Twenty-eight days after tamoxifen only 16±0.9% of SVZ cells, 16±0.6% of GEPCOTs, and 19±1.0% of neurospheres were labeled. This further decreased to 7.5±1.2% of SVZ cells, 12±0.8% of GEPCOTs, and 8±3% of neurospheres at 60 days after tamoxifen (FIG. 2C). This demonstrates that most Ascl1^{CreERT2}-expressing NICs persist in vivo for less than 28 days.

In contrast to cells labeled with Dlx1^{CreERT2} or Ascl1^{CreERT2} cells labeled with Gli1^{CreERT2}, Glast-CreER^{T} , or Sox2^{CreERT2} exhibited a sustained contribution to the pools of SVZ cells, GEPCOTs, and NICs for at least 60 days after tamoxifen administration (FIGS. 2D-F). This suggested the existence of a long-lived NSC population in the SVZ that replenishes NICs and is marked by Gli1^{CreERT2} Glast-CreER^{T}, and Sox2^{CreERT2} expression (FIG. 2G).

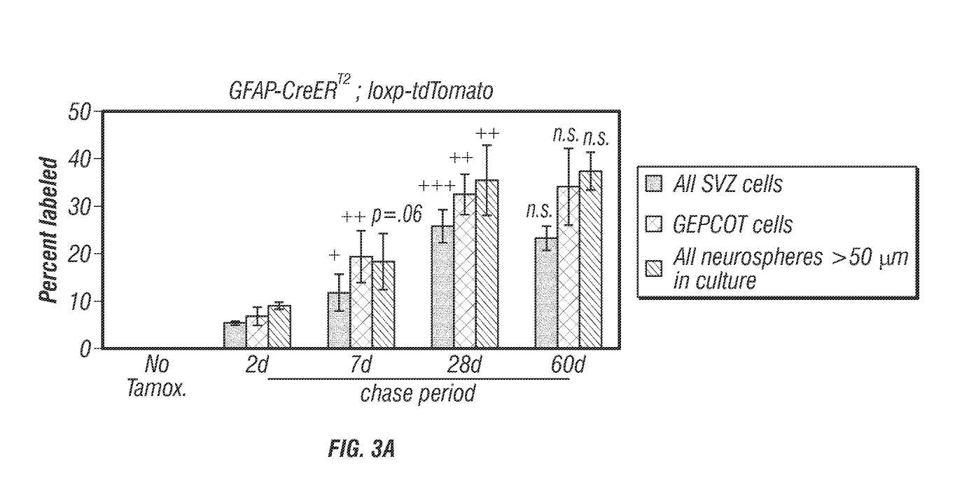

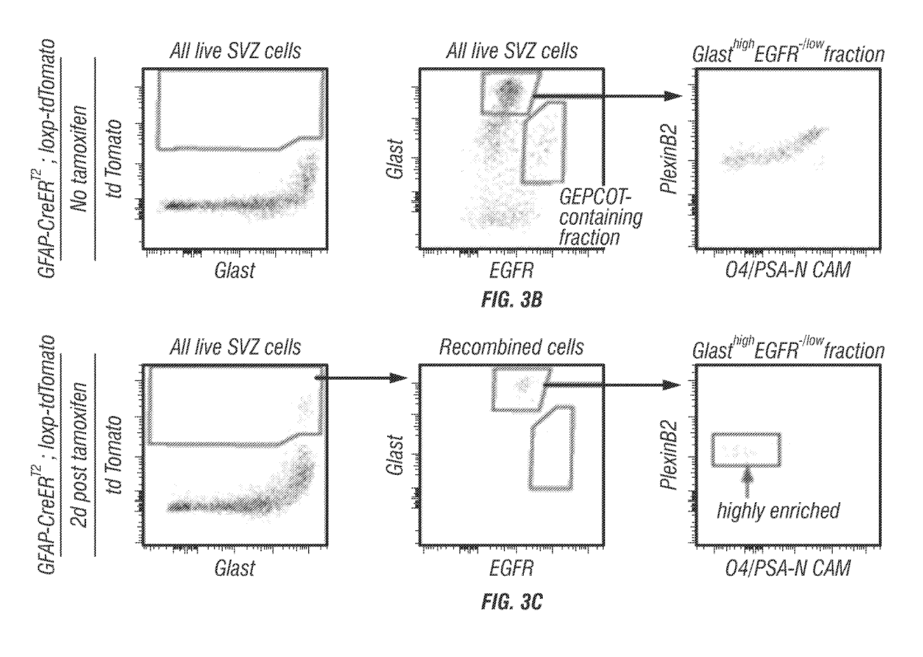

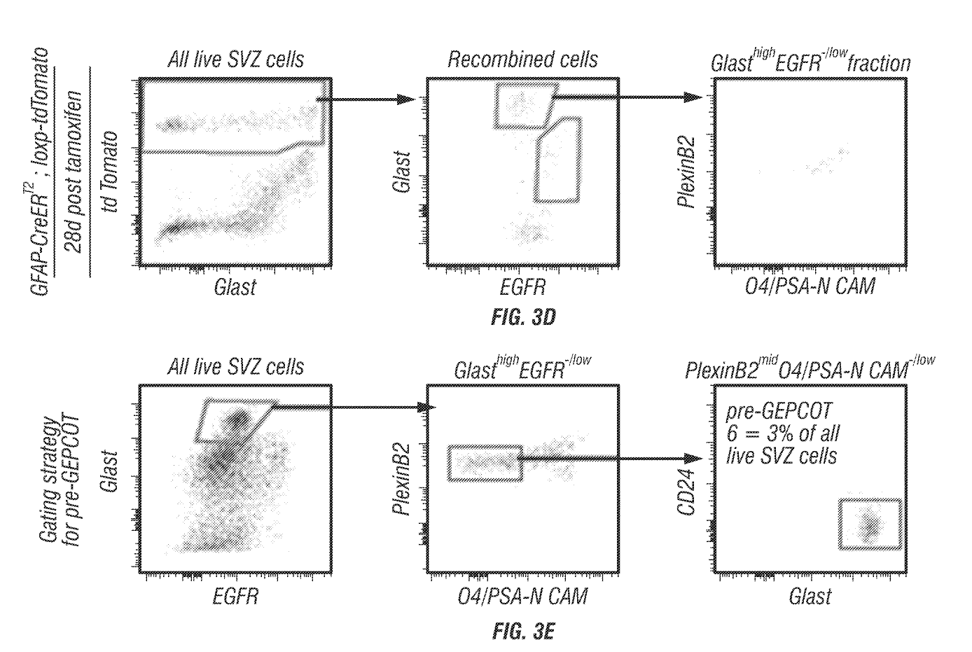

Identification of GFAP-expressing NSCs in vivo. To test whether there is an earlier stem cell population in the SVZ, the inventors sought a CreER^{T2} allele that recombines in long-lived NSCs but not in NICs. Since prior studies have implicated GFAP as a marker of type B cells and NSCs in the adult SVZ (Doetsch et al., 1997; Doetsch et al., 1999a; Doetsch et al., 2002; Mirzadeh et al., 2008; Giachino and Taylor, 2009; Giachino et al., 2013), the inventors performed lineage-tracing experiments with GFAP-CreER^{T2} (Hirrlinger et al., 2006). In the absence of tamoxifen, only 0.02% of SVZ cells, and no GEPCOTs or neurospheres, were tdTomato⁺ (FIG. 3A). Two days after tamoxifen treatment, GFAP-CreER^{T2} labeled only 5±0.6% of SVZ cells, 7±2% of GEPCOTs, and 8±0.9% of neurospheres (FIG. 3A). Seven days after tamoxifen, this increased significantly (p<0.05) to 11±4.3% of SVZ cells, 19±5.5% of GEPCOTs, and 18±6.1% of neurospheres (FIG. 3A). By 28 days after tamoxifen this further increased to 26±3.7% of SVZ cells, 32±4.7% of GEPCOTs, and 35±7.2% of neurospheres (FIG. 3A). At 60 days after tamoxifen, the inventors observed 23±2.6% of SVZ cells, 34±8.0% of GEPCOTs, and 37±4.2% of neurospheres labeled (FIG. 3A). GFAP-CreER^{T2}-expressing NSCs thus make a sustained and growing contribution to the SVZ, in contrast to NICs labeled by Dlx1^{CreERT2} or Ascl1^{CreERT2}.

To better characterize the GFAP-CreER^{T2}-expressing SVZ cells, the inventors examined the surface markers expressed by tdTomato⁺ cells 2 days after tamoxifen treatment of GFAP-CreER^{T2}; loxp-tdTomato mice. By flow cytometry, tdTomato⁺ cells were highly enriched for Glast^{high}EGFR^{-/low}PlexinB2^{mid}CD24^{-/low} O4/PSA-NCAM^{-/low}Ter119/CD45⁻ (pre-GEPCOT) cells (FIGS. 3B-D). These cells differed from GEPCOT cells in that they exhibited higher levels of Glast and lower levels of EGFR and PlexinB2 expression. pre-GEPCOT cells made up only 6±3% of all SVZ cells (FIG. 3E) and yet, based on GFAP-CreER^{T2} lineage tracing, gave rise to many SVZ cells, GEPCOTs, and NICs within 28 days of tamoxifen treatment (FIG. 3A). The GFAP-CreER^{T2}-expressing pre-GEPCOTs therefore included NSCs with a much greater capacity to contribute to the SVZ than Ascl1^{CreERT2}-expressing NICs.

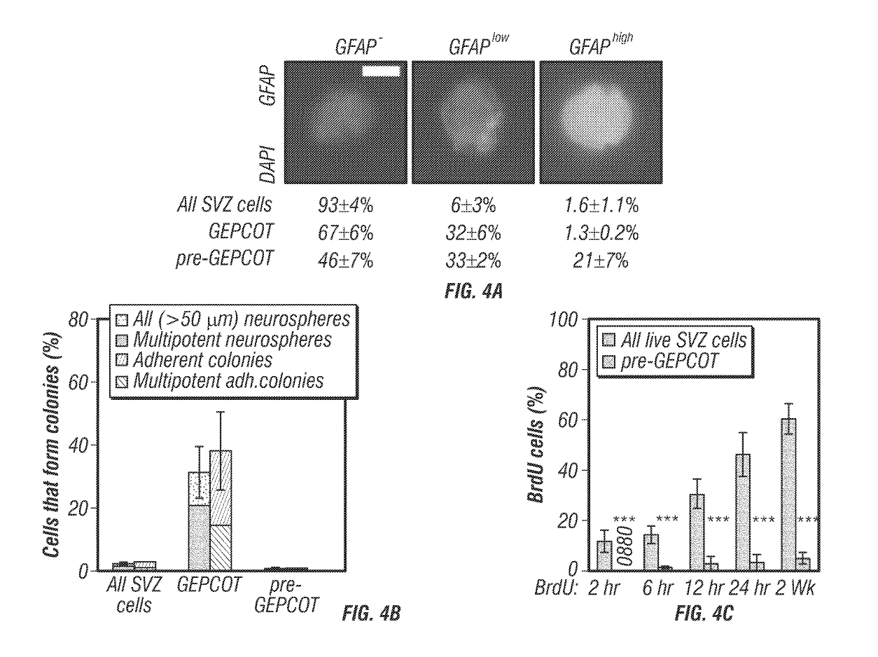

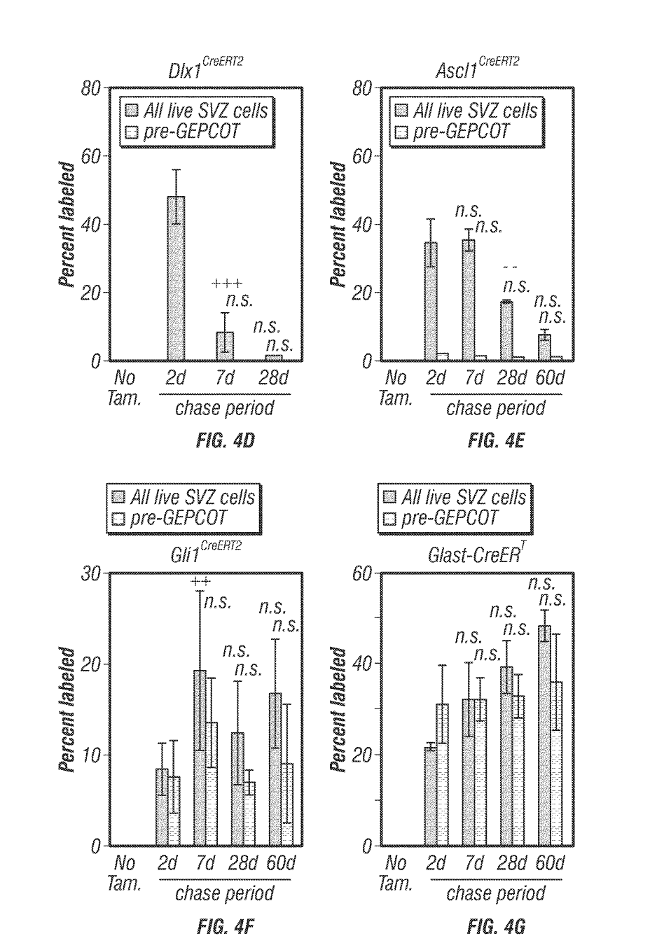

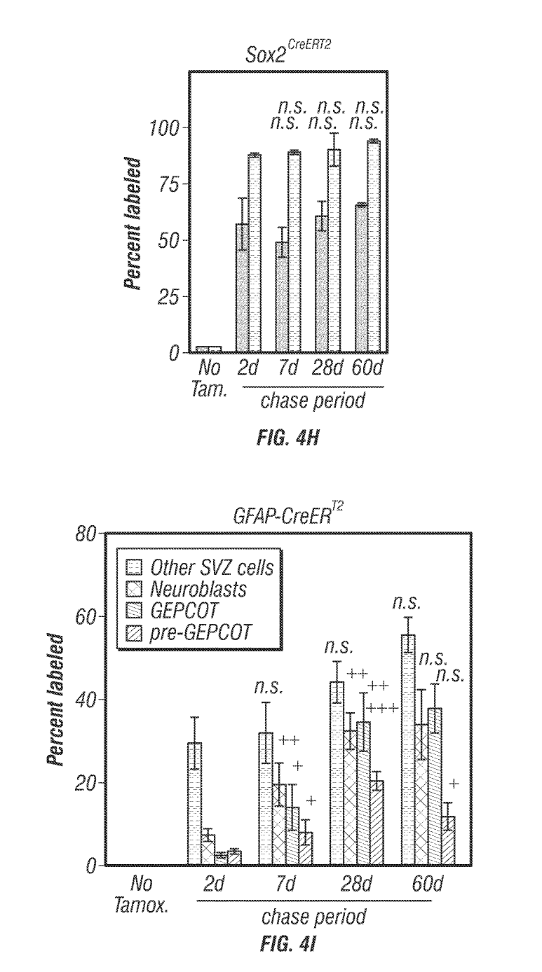

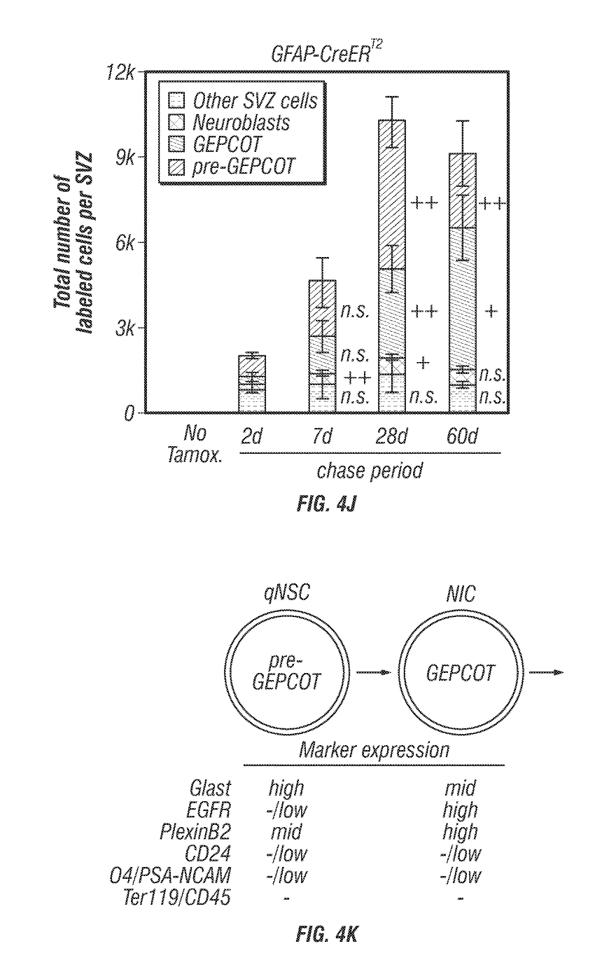

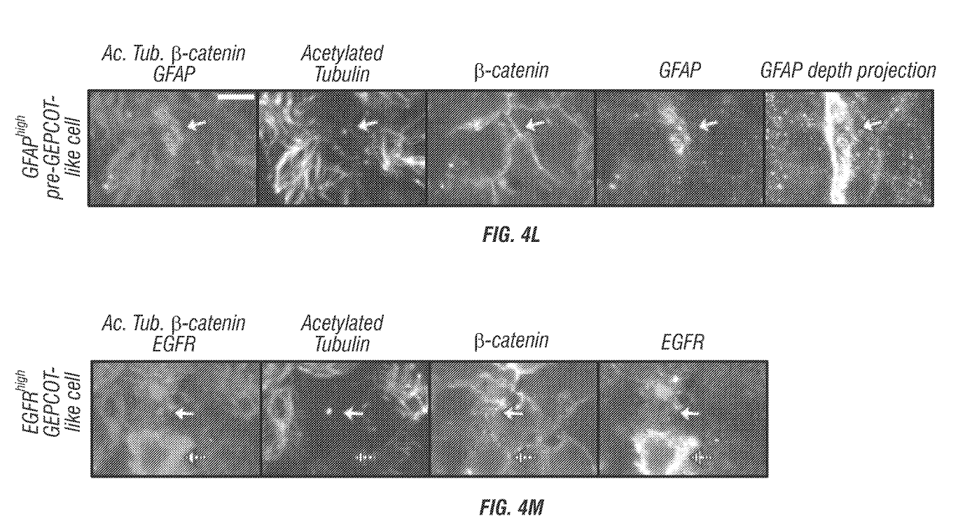

To confirm this, the inventors sorted pre-GEPCOT cells to test whether they stain with an anti-GFAP antibody. GFAP expression was heterogeneous within the pre-GEPCOT population, with 21±7% being GFAP^{high} and 33±2% being GFAP^{low} (FIG. 4A). This suggests that the pre-GEPCOT population is much more highly enriched for NSCs as compared to unfractionated SVZ cells, which contained only 2±1% GFAP^{high} cells and 6±3% GFAP^{low} cells (both p<0.01 relative to pre-GEPCOT cells; FIG. 4A).

pre-GEPCOT cells are long-lived qNSCs. Whereas GEPCOTs were highly enriched for multipotent NICs (FIG. 1C), pre-GEPCOTs were unable to form neurospheres. The inventors sorted unfractionated SVZ cells into culture and found that 1.9±0.5% of the cells formed neurospheres and 2.0±0.4% formed adherent colonies (FIG. 4B). Under the same conditions 32±7% of GEPCOTs formed neurospheres and 38±13% formed adherent colonies (FIG. 4B). In contrast, only very rare pre-GEPCOT cells formed neurospheres (1/1663 cells) and only 0.3±0.4% (9/1973 cells) formed adherent colonies (FIG. 4B).

To assess whether pre-GEPCOT cells are quiescent or frequently dividing in vivo, the inventors administered pulses of BrdU. Although 47±9% of SVZ cells and 89±4% of GEPCOT cells (FIG. 1D) incorporated a 24-hour pulse of BrdU in these experiments, only 3±3% of pre-GEPCOT cells incorporated BrdU in the same mice (FIG. 4C). Similarly, 60±6% of SVZ cells and 91±3% of GEPCOT cells (FIG. 1D) incorporated a 2-week pulse of BrdU in these experiments, but only 4±2% of pre-GEPCOT cells incorporated BrdU (FIG. 4C). These data indicate that pre-GEPCOT cells are quiescent in the SVZ.

To assess whether fate-mapping with CreER^{T2} alleles is consistent with the existence of a pre-GEPCOT qNSC population, the inventors assessed whether they recombined in pre-GEPCOT cells. Dlx1^{CreERT2}, which labeled 51±6% of all SVZ cells and 15±6% of GEPCOTs at 2 days after tamoxifen treatment (FIG. 2B), did not label any pre-GEPCOT cells at 2, 7, or 28 days after tamoxifen treatment (FIG. 4D). Ascl1^{CreERT2}, which labeled 34±7% of all SVZ cells and 63±8% of GEPCOTs at 2 days after tamoxifen (FIG. 2C), labeled fewer than 1% of pre-GEPCOT cells at 2, 7, 28, and 60 days after tamoxifen (FIG. 4E). Thus, both of the CreER^{T2} alleles that exhibited declining contributions to the SVZ upon fate-mapping labeled GEPCOTs but not pre-GEPCOTs. Neither the Dlx1^{CreERT2}-expressing GEPCOT NICs nor the Ascl1^{CreERT2}-expressing GEPCOT NICs gave rise to significant numbers of pre-GEPCOT qNSCs in vivo, at least under steady-state conditions.

To assess whether pre-GEPCOTs contain qNSCs, the inventors tested whether the CreER^{T2} alleles that sustainably contributed to the SVZ over time (Gli1^{CreERT2}, Glast-CreER^{T}, and Sox2^{CreERT2}) labeled pre-GEPCOT cells. Gli1^{CreERT2} which sustainably labeled 8-19% of all SVZ cells and 26-40% of GEPCOTs for 60 days after tamoxifen treatment (FIG. 2D), also sustainably labeled 7-13% of pre-GEPCOT cells at 2 to 60 days after tamoxifen treatment (FIG. 4F). Glast-CreER^{T}, which sustainably labeled 21-48% of all SVZ cells and 61-72% of GEPCOTs for 60 days after tamoxifen (FIG. 2E), also labeled 31-36% of pre-GEPCOT cells 2 to 60 days after tamoxifen (FIG. 4G). Sox2^{CreERT2} which sustainably labeled 49-65% of all SVZ cells and 92-97% of GEPCOTs for 60 days after tamoxifen (FIG. 2F), also sustainably labeled 87-93% of pre-GEPCOT cells 2 to 60 days after tamoxifen (FIG. 4H). Thus all of the CreER^{T2} alleles that gave a sustained contribution to NIC and SVZ labeling also labeled pre-GEPCOTs, consistent with the suggestion that pre-GEPCOT cells contain qNSCs. Moreover, every CreER^{T2} allele that labeled pre-GEPCOT cells exhibited sustained labeling of not only pre-GEPCOT cells but also GEPCOTs, NICs, and unfractionated SVZ cells.

The inventors tested whether the GFAP-CreER^{T2} recombination pattern was consistent with pre-GEPCOT qNSCs giving rise to GEPCOT NICs in vivo. Two days after tamoxifen administration to GFAP-CreER^{T2}; loxp-tdTomato mice, the tdTomato label was present mainly in pre-GEPCOT cells. On average, 29±7% of pre-GEPCOT cells were tdTomato⁺ whereas only 6.7±1.9% of GEPCOT cells, 2.2±0.5% of PSA-NCAM^{high}CD24^{mid} neuroblasts (Pastrana et al., 2009), and 3.0±0.8% of other SVZ cells were labeled (FIG. 41). These data indicate that GFAP-CreER^{T2} recombines efficiently in pre-GEPCOTs but not GEPCOT NICs in vivo. Twenty-eight days after tamoxifen treatment, the percentage of labeled pre-GEPCOT cells increased to 44±5.8% of cells (FIG. 41). The percentages of labeled GEPCOTs, neuroblasts, and other SVZ cells also increased significantly (p<0.01) by 28 days to 32±4.7%, 35±6.8%, and 20±2.2%, respectively. At 60 days after tamoxifen, the inventors continued to observe strong labeling in pre-GEPCOTs, GEPCOTs, and neuroblasts (FIG. 4I). Similar trends were apparent when the data were expressed in absolute numbers (FIG. 4J). These data suggest GFAP-CreER^{T2}-expressing pre-GEPCOT cells include qNSCs that give rise to NICs, neuroblasts, and other SVZ cells (FIG. 4K).

The inventors examined the localization of pre-GEPCOT and GEPCOT cells in whole mount stains of the SVZ. pre-GEPCOTs were distinguished by GFAP expression, a marker of type B cells (Doetsch et al., 1999a) and GEPCOTs were distinguished by EGFR expression, a marker of type C cells (Doetsch et al., 2002). The inventors stained whole-mount SVZs with antibodies against acetylated tubulin and β-catenin to detect “pinwheel” structures associated with type B1 stem cells in the SVZ (Mirzadeh et al., 2008) then also stained with antibodies against GFAP and EGFR. Consistent with prior results (Mirzadeh et al., 2008), the inventors observed GFAP^{high} cells at the center of the pinwheel structures with a single primary cilium on the apical surface contacting the ventricle and a long basal process (FIG. 4L). Since many pre-GEPCOTs recombined with GFAP-CreER^{T2} but virtually no GEPCOTs did (FIG. 4I), these data indicate that many pre-GEPCOT cells have the morphology and position of type B1 cells in the SVZ. In contrast, EGFR^{high} cells had type C cell morphology: round, unciliated, and generally not contacting the ventricle, consistent with Doetsch et al. (2002) (FIG. 4M). Since all GEPCOT cells express high levels of EGFR (FIG. 1B) while pre-GEPCOT cells have low levels of EGFR (FIG. 3D), these data suggest that many GEPCOT cells have the morphology and position of type C cells.

Anti-mitotic agent eliminates GEPCOT NICs but not pre-GEPCOT qNSCs. The inventors' observation that pre-GEPCOT cells include qNSCs raised the question of whether pre-GEPCOT cells are resistant to anti-mitotic agents. To test this, the inventors treated mice for 3 consecutive days with the CNS-penetrating DNA-alkylating agent temozolomide (TMZ; 100 mg/kg/day i.p.) (Garthe et al., 2009) then assessed SVZ proliferation, composition, and neurosphere formation 3 to 90 days later (FIG. 5A). Three days after TMZ, mice appeared healthy and had lost less than 3% of their body mass as compared to before TMZ treatment (data not shown). Three days after TMZ treatment, the inventors observed a 23% reduction in the total number of cells per SVZ and this reduction remained nearly constant over the next 90 days (FIG. 5B). Three days after TMZ, the inventors observed an 80% reduction in the number of dividing SVZ cells, based on BrdU incorporation (FIG. 5C, p<0.001). The number of dividing SVZ cells slowly increased over time, recovering to 50% of normal by 90 days after TMZ treatment (FIG. 5C). Three days after TMZ only 0.7% of the NICs observed in saline-treated control mice remained in TMZ-treated mice (FIG. 5D, p<0.001). By 16 days after TMZ treatment NICs recovered to 20% of normal levels (FIG. 5D, p<0.001). The numbers of NICs continued to increase over time, recovering to 55% of normal by 90 days after TMZ (FIG. 5D, p<0.001). NICs are thus virtually completely eliminated by TMZ but regenerate over time as expected.

Consistent with the effects of TMZ on NICs, TMZ treatment also eliminated nearly all GEPCOT cells. Three days after TMZ treatment, only 10% of the GEPCOTs observed in saline-treated control mice remained in TMZ treated mice (FIG. 5E, p<0.001). By 16 days after TMZ treatment, GEPCOTs recovered to 32% of normal levels (FIG. 5E, p<0.001). GEPCOTs recovered to 55% of normal levels by 90 days after TMZ treatment (FIG. 5E, p<0.001).

The numbers of pre-GEPCOT cells in the SVZ were not affected by TMZ treatment (FIG. 5F). If pre-GEPCOT qNSCs regenerate NICs after TMZ treatment then serial TMZ treatment might be expected to deplete pre-GEPCOT cells. To test this, the inventors administered two rounds of TMZ 12 days apart (FIG. 5G). Under these circumstances, pre-GEPCOT cells were not depleted 3 days after the second round of TMZ treatment but they were depleted by 90 days after the second round of TMZ (FIG. 5L). Consistent with the observation that pre-GEPCOT cells were sensitive to serial TMZ treatment, the inventors detected little recovery of dividing SVZ cells (FIG. 5I), NICs (FIG. 5J), or GEPCOTs (FIG. 5K) after serial TMZ treatment. These data suggest that GEPCOT NICs are replenished after ablation by pre-GEPCOT qNSCs.

To directly test whether GEPCOTs arise from pre-GEPCOT cells during SVZ regeneration, the inventors performed lineage tracing using multiple Cre alleles by first labeling cells with tamoxifen and then treating with TMZ (FIG. 5M). After recombination of a conditional reporter with the GEPCOT marker Ascl1^{CreERT2} the number of unlabeled GEPCOTs per SVZ increased from 120±50 at 3 days after TMZ to 640±210 at 35 days after TMZ (p<0.01) but Ascl1^{CreERT2}-labeled GEPCOTs did not significantly change (FIG. 5N). The GEPCOTs, NICs, and neuroblasts that regenerated after TMZ treatment thus did not arise from Ascl1^{CreERT2}-labeled GEPCOTs (FIG. 5N). In contrast, after recombination of a conditional reporter with the pre-GEPCOT markers Glast-CreER^{T} and GFAP-CreER^{T2}, the inventors observed significant (p<0.05) increases in the frequencies of labeled GEPCOT cells, NICs, and neuroblasts between 3 and 35 days after TMZ treatment (FIGS. 50-P). These results strongly suggest that pre-GEPCOT cells give rise to GEPCOTs, NICs, and neuroblasts during SVZ regeneration.

Bmi-1 is required by pre-GEPCOT qNSCs and GEPCOT NICs in vivo. Pre-GEPCOT qNSCs and GEPCOT NICs expressed similar levels of Bmi-1 by qRT-PCR (FIG. 6A). To study the molecular mechanisms that regulate the maintenance of pre-GEPCOT qNSCs and GEPCOT NICs in vivo, the inventors generated a floxed allele of Bmi-1 (FIGS. 10A-B) and conditionally deleted Bmi-1 using Nestin-Cre (Tronche et al., 1999). This allele of Nestin-Cre deletes broadly throughout the neuroepithelium by E10.5 such that there is nearly homogeneous recombination in the postnatal CNS (word-wide-web at cre.jax.org/Nes/Nes-CreNano.html). Consistent with this, Nestin-Cre; Bmi-1^{fl/fl} mice exhibited a loss of Bmi-1 protein in the cortex and SVZ by western blot (FIG. 6B) and a loss of Bmi-1 transcripts in SVZ cells, GEPCOT cells, and pre-GEPCOT cells by qRT-PCR (FIG. 10G).

Nestin-Cre; Bmi-1^{fl/fl} mice were modestly but significantly smaller than littermate controls (FIG. 10C). Unlike germline Bmi-1 deficient mice, which generally die within a month after birth (van der Lugt et al., 1994; Lessard and Sauvageau, 2003; Park et al., 2003; Bruggeman et al., 2005; Molofsky et al., 2005), the inventors were able to age Nestin-Cre; Bmi-1^{fl/fl} mice for up to 2 years. These mice did exhibit neurological deficits that worsened during aging, such as ataxia, as was reported in germline Bmi-1 deficient mice (van der Lugt et al., 1994). However, the survival of Nestin-Cre; Bmi-1^{fl/fl} mice throughout adulthood made it possible for the first time to test whether Bmi-1 is autonomously required by qNSCs or NICs in the adult brain.

Consistent with the phenotype of germline Bmi-1 deficient mice (van der Lugt et al., 1994; Bruggeman et al., 2005; Molofsky et al., 2005), brain morphology appeared relatively normal in Nestin-Cre; Bmi-1^{fl/fl} mice (FIG. 10E). Brain size was slightly but significantly reduced in Nestin-Cre; Bmi-1^{fl/fl} mice as compared to littermate controls (FIG. 10D) but was normal as a proportion of body mass (data not shown). The cerebellums from Nestin-Cre; Bmi-1^{fl/fl} mice were smaller than in control mice and had significantly thinner molecular layers (FIGS. 6D-E). The inventors also observed unusually prominent GFAP staining throughout the cortex of adult Nestin-Cre; Bmi-1^{fl/fl} mice (FIG. 10F). Otherwise, the olfactory bulb, hippocampus, and cortex appeared grossly normal (FIG. 10E), though additional work will be required to carefully assess laminar organization and identity in each brain region. Our data suggest that the smaller cerebellums and prominent GFAP staining reflect a cell-autonomous requirement for Bmi-1 in fetal neural stem/progenitor cells.

The inventors observed complete Bmi-1 recombination in sorted SVZ cells, GEPCOT cells, and pre-GEPCOT cells isolated from adult Nestin-Cre; Bmi-1^{fl/fl} mice (FIG. 6F). Twelve month-old Nestin-Cre; Bmi-1^{fl/fl} mice exhibited a significantly reduced number of SVZ cells that incorporated a 2-hour pulse of BrdU (FIG. 6G). The inventors measured the rate of neurogenesis in Nestin-Cre; Bmi-1^{fl/fl} mice and Bmi-1^{fl/fl} controls by administering BrdU for 7 days (beginning at 4 or 14 months of age) followed by a 4-week chase without BrdU. The inventors quantified the frequency of BrdU⁺NeuN⁺ newborn neurons in sections from the olfactory bulb by microscopy. Bmi-1 deletion had no effect on neurogenesis at 4 months of age, but significantly reduced olfactory bulb neurogenesis in 14-month-old mice (FIG. 6H, p<0.01).

Neurospheres formed by Nestin-Cre; Bmi-1^{fl/fl} SVZ cells were significantly smaller than control neurospheres (FIG. 6I), were unable to undergo multilineage differentiation (FIG. 6J), and could not be passaged (FIG. 6K). In contrast, control neurospheres readily underwent multilineage differentiation (FIG. 6J) and were able to generate multipotent daughter neurospheres upon subcloning into secondary cultures (FIG. 6K).

To assess whether Bmi-1 deficiency affected pre-GEPCOT qNSCs or GEPCOT NICs in vivo, the inventors assessed the frequencies of these cells in SVZs from 4 and 12 month old Nestin-Cre; Bmi-1^{fl/fl} mice and littermate controls. The frequency of pre-GEPCOT cells was significantly reduced at 4 months of age (The inventors 6L). The inventors observed no significant effects on the frequencies of GEPCOT cells (FIG. 6M) or neuroblasts (FIG. 6N) at 4 months of age. However, each of these populations was significantly depleted in 12 month old Nestin-Cre; Bmi-1^{fl/fl} mice (FIGS. 6L-N). Bmi-1 is thus required for the maintenance of normal numbers of pre-GEPCOT qNSCs and GEPCOT NICs in the adult brain. Nonetheless, some pre-GEPCOT cells, GEPCOT cells, and neurogenesis did persist for at least a year in adult mice, demonstrating that NSCs are not absolutely dependent upon Bmi-1 for their maintenance in the adult brain.

Bmi-1 promotes the maintenance of neural stem cells partly by repressing the Cdkn2a (p16^{Ink4a}/p19^{Arf}) locus (Jacobs et al., 1999; Molofsky et al., 2003; Molofsky et al., 2005; Bruggeman et al., 2007). The inventors were unable to detect p16^{Ink4a} transcripts in wild-type SVZ cells, pre-GEPCOT qNSCs, or GEPCOT NICs in 4-month-old (0/5) or 12 month old (0/5) mice (FIG. 6O). In contrast, 4-month old Bmi-1-deficient SVZ cells (5/5 mice) and pre-GEPCOT cells (⁴/₅ mice) did express p16^{Ink4a} and the level of p16^{Ink4a} expression significantly increased between 4 and 12 months of age (FIG. 6O; detected in 5/5 mice for both populations). Low level p16^{Ink4a} expression was detected in GEPCOT cells from two of five 4 month-old Bmi-1-deficient mice and in GEPCOT cells from one of five 12 month old Bmi-1-deficient mice (FIG. 6O). Bmi-1 is thus required to repress p16^{Ink4a} expression in pre-GEPCOT qNSCs.

Bmi-1 promotes adult neurogenesis and gliogenesis in vivo. To conditionally delete Bmi-1 in the adult brain the inventors used Nestin-CreER^{T2}, which deletes broadly throughout the SVZ, including within qNSCs that give rise to SVZ cells after AraC treatment (Giachino and Taylor, 2009). The inventors observed significantly lower levels of Nestin transcripts within pre-GEPCOT qNSCs as compared to GEPCOT NICs (FIG. 11A) and only 9.3±7.3% of pre-GEPCOTs stained positively for Nestin protein as compared to 89.7±8.5% of GEPCOTs and 39.0±5.3% of SVZ cells (FIG. 11B). The inventors also observed higher levels of Nestin transgene expression in GEPCOT cells as compared to pre-GEPCOT cells, including Nestin-mCherry and Nestin-GFP (FIGS. 11C-D). However, when the inventors analyzed recombination of a conditional reporter in Nestin-CreER^{T2} mice, the inventors observed labeling of 99±1.1% of GEPCOT cells and 96±1.9% of pre-GEPCOT cells (FIG. 11E). Therefore, although endogenous Nestin is expressed at lower levels in qNSCs as compared to NICs, Nestin second intronic enhancer transgenes are variably expressed in both pre-GEPCOT qNSCs and GEPCOT NICs, and Nestin-CreER^{T2} gives nearly complete recombination in both cell populations.

The inventors administered tamoxifen in the chow of Nestin-CreER^{T2}; Bmi-1^{fl/fl} mice and littermate controls for 30 days beginning at 6 weeks of age and assessed recombination efficiency 2 weeks after completing tamoxifen treatment (that is, starting at 3 months of age). Polymerase chain reaction (PCR) analysis of genomic DNA from individual neurospheres revealed complete deletion of Bmi-1 in at least 95% of neurospheres (FIGS. 12A-B). Western blot analysis demonstrated a near total loss of Bmi-1 protein from neurospheres (FIG. 7A), consistent with the high rate of recombination in individual neurospheres. By PCR analysis of genomic DNA, the inventors observed near complete recombination in unfractionated SVZ cells and complete recombination in pre-GEPCOT and GEPCOT cells isolated from adult Nestin-CreER^{T2}; Bmi-1^{fl/fl} mice (FIG. 7B).

Body mass (FIG. 12C), brain histology (data not shown), and cerebellum size (FIG. 12D) were all grossly normal in Nestin-CreER^{T2}; Bmi-1^{fl/fl} mice six months after tamoxifen treatment. To assess the consequences of Bmi-1 deletion from adult neural stem/progenitor cells, the inventors measured the rate of neurogenesis in Nestin-CreER^{T2}; Bmi-1^{fl/fl} mice and Bmi-1^{fl/fl} controls by administering BrdU for 7 days (beginning at 2 weeks, 6 months and 12 months after tamoxifen treatment, at which time the mice were approximately 3 months old, 9 months old and 15 months old, respectively) followed by a 4-week chase without BrdU. Bmi-1 deficiency had no effect on the frequency of BrdU⁺NeuN⁺ newborn neurons in the olfactory bulb 2 weeks after tamoxifen treatment but significantly reduced neurogenesis 6 and 12 months after tamoxifen (FIG. 7C, p<0.05). The reduced neurogenesis in Bmi-1 mutant mice was evident in all subsets of neurons that the inventors investigated, including BrdU⁺Calretinin⁺ neurons, BrdU⁺Calbindin⁺ neurons, and BrdU⁺Tyrosine Hydroxylase⁺ neurons (FIGS. 7D-E). Bmi-1 is therefore required in adult neural stem/progenitor cells for normal neurogenesis but Bmi-1 deficiency did not completely eliminate the generation of forebrain neurons during the first year of life.

Consistent with prior studies, the vast majority of the newborn cells in the adult olfactory bulb were neurons, but the small numbers of astrocytes that arose in the olfactory bulb and oligodendrocytes that arose in the cortex were also diminished after Bmi-1 deletion. The frequency of newborn olfactory bulb BrdU⁺S1001β⁺ astrocytes appeared normal 6 months after tamoxifen treatment but was significantly reduced 12 months after tamoxifen in Bmi-1 mutant mice (FIG. 7F). The frequency of newborn cortical BrdU⁺GST-pi⁺ oligodendrocytes declined 6 months after tamoxifen treatment and could no longer be detected after 12 months (FIG. 7G). The deficits in neurogenesis and gliogenesis became worse over time after Bmi-1 deletion.

The inventors quantified the frequency of SVZ cells that incorporated a 2-hour pulse of BrdU. Bmi-1 mutant mice had significant reductions in the frequencies of BrdU⁺ SVZ cells at 2 weeks (50% reduction, p<0.02), 6 months (55% reduction, p<0.02) and 12 months (90% reduction, p<0.001) after tamoxifen treatment (FIG. 7H). Bmi-1 mutant mice also had significant (p<0.001) reductions in the frequencies of Mcm2⁺ (FIGS. 12E, 1G) and Ki67⁺ (FIG. 12F) SVZ cells. Consistent with this, Bmi-1 mutant mice exhibited significant (p<0.001) declines in the frequencies of Dcx⁺ (FIG. 7I) and CD24^{mid}PSA-NCAM⁺ neuroblasts (FIG. 7J) 6 and 12 months after tamoxifen treatment.

GEPCOT NICs acutely depend upon Bmi-1 in the adult SVZ. To assess whether Bmi-1 is required by adult NICs, the inventors plated SVZ cells from tamoxifen-treated Nestin-CreER^{T2}; Bmi-1^{fl/fl} and control mice in non-adherent cultures and assessed GEPCOT cell frequency. Two weeks after tamoxifen treatment the frequencies of NICs (FIG. 7K) and GEPCOTs (FIG. 7Q) were unchanged in Bmi-1 mutant mice as compared to littermate controls. However, the Bmi-1-deficient neurospheres were significantly smaller than control neurospheres (FIGS. 7L-M) and did not undergo multilineage differentiation (FIG. 7N; they formed only astrocytes upon transfer to adherent cultures) or self-renew upon subcloning (FIG. 7O). At six and twelve months after tamoxifen treatment, Bmi-1 deficient SVZ cells formed virtually no neurospheres (FIG. 7K). Consistent with this, Nestin-CreER^{T2}; Bmi-1^{fl/fl} mice also had significantly lower frequencies of GEPCOTs than control mice at 6 months and 12 months after tamoxifen treatment, and the magnitude of the depletion increased over time (FIG. 7Q). Virtually no GEPCOTs could be found in Nestin-CreER^{T2}; Bmi-1^{fl/fl} mice 12 months after tamoxifen treatment (FIG. 7Q). These data demonstrate that GEPCOT NICs require Bmi-1 in vivo for their maintenance in the adult SVZ, though the cells are able to persist for several months after Bmi-1 deletion before they are completely eliminated.

To test whether qNSCs depend upon Bmi-1 for their maintenance in vivo, the inventors examined the frequency of pre-GEPCOT cells in tamoxifen-treated Nestin-CreER^{T2}; Bmi-1^{fl/fl} mice and littermate controls. pre-GEPCOT cells were not depleted at two weeks or 6 months after tamoxifen treatment (FIG. 7P). The inventors observed a clear trend toward reduced pre-GEPCOT cell frequency in Nestin-CreER^{T2}; Bmi-1^{fl/fl} mice 12 months after tamoxifen treatment but the effect was not statistically significant.

To test whether there was a loss of Bmi-1 function in these cells, the inventors performed qRT-PCR to assess p16^{Ink4a} expression in cells from Nestin-CreER^{T2}; Bmi-1^{fl/fl} mice. One mechanism by which Bmi-1 promotes the maintenance of neural stem/progenitor cells is by negatively regulating p16^{Ink4a} expression (Jacobs et al., 1999; Molofsky et al., 2003; Bruggeman et al., 2005; Molofsky et al., 2005). The inventors did not detect p16^{Ink4a} transcripts in wild-type SVZ cells, pre-GEPCOT qNSCs, or GEPCOT NICs 2 weeks (0/5), 3 months (0/6), or 8 months (0/7) after tamoxifen treatment (FIG. 7R). The inventors rarely detected p16^{Ink4a} transcripts in SVZ cells or GEPCOT cells from Nestin-CreER^{T2}; Bmi-1^{fl/fl} mice 2 weeks (0/5 SVZ, 0/5 GEPCOT), 3 months (0/5 SVZ, 1/5 GEPCOT) or 8 months (1/9 SVZ, 0/9 GEPCOT) after tamoxifen treatment. p16^{Ink4a} transcripts were rarely detected in pre-GEPCOT cells from Nestin-CreER^{T2}; Bmi-1^{fl/fl} mice 2 weeks (0/5) or 3 months (1/5) after tamoxifen treatment. However, 8 months after tamoxifen treatment p16^{Ink4a} expression was detected in pre-GEPCOT cells from seven of nine Nestin-CreER^{T2}; Bmi-1^{fl/fl} mice (FIG. 7R, FIG. 12H). These data demonstrate that Bmi-1 is required during adulthood to negatively regulate p16^{Ink4a} expression in pre-GEPCOT qNSCs but that these cells are depleted more slowly than GEPCOT NICs in the adult SVZ. Although high levels of p16^{Ink4a} expression are associated with cellular senescence, the inventors were unable to detect any increase in senescence associated β-galactosidase activity in pre-GEPCOT cells from 14 month old Nestin-Cre; Bmi-1^{fl/fl} mice or in cultured neural stem/progenitor cells from Bmi-1 germline deficient mice (data not shown).

### Example 3

### Discussion

By screening almost four hundred antibodies against distinct cell surface antigens, the inventors identified two phenotypically and functionally distinct populations of neural stem/progenitor cells from the adult mouse SVZ. GEPCOT cells were highly enriched for NICs (FIG. 1C) and highly mitotically active in vivo (FIG. 1D) but persisted only transiently in the SVZ based on fate mapping with Ascl1^{CreERT2 or Dlx}1^{CreErT2} (FIGS. 2B-C). In contrast, pre-GEPCOT cells lacked the ability to form neurospheres or adherent colonies in culture (FIG. 4B), and were quiescent in vivo (FIG. 4C) but were long-lived in the SVZ based on fate mapping with the stem cell markers Glast-CreER^{T} (Wang et al., 2012), GFAP-CreER^{T2} (Giachino et al., 2013), Sox2^{CreERT2} (Arnold et al., 2011), and Gli1^{CreERT2} (Ahn and Joyner, 2005; Lee et al., 2012) (FIGS. 4F-J). In contrast to GEPCOT NICs, pre-GEPCOT cells were resistant to TMZ (FIGS. 5E-F). Although TMZ eliminated virtually all NICs from the SVZ (FIG. 5D), the dividing cells, NICs, and GEPCOTs regenerated within a month of TMZ treatment (FIG. 5). The persistence of pre-GEPCOT cells after TMZ (FIG. 5F), and the regeneration of GEPCOTs from a precursor that expresses Glast-CreER^{T} and GFAP-CreER^{T2} but not Ascl1^{CreERT2} (FIGS. 5N-P) suggest that the SVZ regenerates from pre-GEPCOT qNSCs.

The conclusion that pre-GEPCOT cells include qNSCs is also supported by the sustained contribution of GFAP-CreER^{T2} marked cells to the SVZ, as GFAP-CreER^{T2} labeled many pre-GEPCOT cells but few GEPCOT cells or NICs immediately after tamoxifen treatment (FIG. 3A, FIG. 4I). Thus, these data demonstrate that GFAP-CreER^{T2} recombination is a marker of qNSCs that survive treatment with anti-mitotic agents and contribute to an increasing proportion of pre-GEPCOT cells, GEPCOT cells, and neuroblasts over time (FIGS. 41, 5M-P).

NSCs are commonly estimated to account for a few percent of cells in germinal zones in the adult mouse brain (Doetsch et al., 1997; Mirzadeh et al., 2008; Pastrana et al., 2009). Although pre-GEPCOT cells accounted for 6±3% of SVZ cells, this population is likely to be heterogeneous, consistent with its heterogeneous GFAP expression (FIG. 4A). Thus, the actual frequency of qNSCs in the SVZ may be lower than 6%. The current lack of a clonal assay for qNSCs makes it impossible to test the purity of this population. In an effort to identify culture conditions permissive for colony formation by pre-GEPCOT cells, the inventors screened 36 different growth factors or medium supplements but none significantly increased colony formation by SVZ cells from normal or TMZ-treated mice (data not shown). Thus, future studies will be required to develop culture conditions that permit efficient colony formation by individual qNSCs and to isolate these cells at high purity.

An interesting question for future studies will be whether there are regional differences in the distributions or properties of pre-GEPCOT qNSCs or GEPCOT NICs within the SVZ, corresponding to regional differences in stem/progenitor cell properties (Merkle et al., 2007; Ihrie et al., 2011; Merkle et al., 2014).

Germline deficiency for the polycomb family member Bmi-1 eliminates multipotent NICs and reduces proliferation and neurogenesis in the SVZ (Molofsky et al., 2003; Bruggeman et al., 2005; Molofsky et al., 2005; Zencak et al., 2005; Bruggeman et al., 2007; Fasano et al., 2009). However, the lack of a floxed allele of Bmi-1, the death of germline Bmi- 1-deficient mice before adulthood, and the lack of prospective markers for NSCs made it impossible to directly test whether Bmi-1 was required for the maintenance of adult NSCs in vivo.

Conditional deletion of Bmi-1 from the fetal and adult SVZ using Nestin-Cre (FIG. 6) and Nestin-CreER^{T2} (FIG. 7) demonstrated that pre-GEPCOT qNSCs and GEPCOT NICs require Bmi-1 to be maintained in normal numbers in the adult forebrain, but the depletion of these cells and the loss of neurogenesis occurred much more gradually than expected. Even when Bmi-1 was completely deleted in neural stem/progenitor cells during fetal development, normal or near-normal numbers of pre-GEPCOT cells, GEPCOT cells, and SVZ neuroblasts were found in the SVZ of 4 month old Nestin-Cre; Bmi-1^{fl/fl} mice (FIGS. 6L-N). This demonstrates that pre-GEPCOT qNSCs and GEPCOT NICs can persist throughout fetal and postnatal development, and into adulthood, in the absence of Bmi-1. This suggests that qNSCs and NICs are less acutely dependent upon Bmi-1 for their maintenance than expected based on studies of Bmi-1 germline knockout mice (van der Lugt et al., 1994; Molofsky et al., 2003; Bruggeman et al., 2005; Molofsky et al., 2005; Fasano et al., 2009).

There are likely several reasons why Bmi-1 germline knockout mice appeared to exhibit a more severe loss of neural stem/progenitor cells and neurogenesis than is evident in conditional knockout mice. First, the phenotype in Bmi-1 germline knockout mice may indeed be more severe as non-cell-autonomous effects of Bmi-1-deficient stroma contribute to stem cell deficits in these mice (Oguro et al., 2006; Oguro et al., 2010). Second, the death of Bmi-1 germline knockout mice before young adulthood meant that adult phenotypes could not be studied directly and it was assumed that the deficits observed in the early postnatal period would rapidly worsen over time. Third, without the ability to prospectively identify qNSCs or NICs in vivo, these cells could only be studied based on neurosphere formation in culture. Since p16^{Ink4a} and p19^{Arf} expression are induced to a greater extent in culture than in vivo, the severity of Bmi-1 deficiency phenotypes in the nervous system are sometimes exaggerated in culture (Molofsky et al., 2005; He et al., 2009). Our data indicate that Bmi-1 is not required for the persistence of NSCs or neurogenesis into adulthood, though both are depleted over time during adulthood after Bmi-1 deletion.

Consistent with the gradual depletion of pre-GEPCOT cells, GEPCOT cells, and neurogenesis during adulthood after conditional Bmi-1 deletion, the inventors also observed a gradual increase in p16^{Ink4a} expression in pre-GEPCOT cells but not in GEPCOT cells (FIGS. 6O, 7R, 12H). This suggests that qNSCs are particularly dependent upon Bmi-1 for the repression of p16^{Ink4a} expression.

The identification of markers that prospectively identify and isolate distinct populations of qNSCs and NICs will make it possible to characterize their properties in vivo rather than relying upon colony-forming assays in culture. Use of commercially available antibodies against cell surface antigens will enable such studies in a wide range of genetic backgrounds. The inventors' data on Bmi-1 demonstrate how the existence of these markers makes it possible to assess the function of gene products within the neural stem/progenitor cell pool with a more granular appreciation for effects on qNSCs versus NICs.

Although the present disclosure and its advantages have been described in detail, it should be understood that various changes, substitutions and alterations can be made herein without departing from the disclosure as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

### REFERENCES

The following references, to the extent that they provide exemplary procedural or other details supplementary to those set forth herein, are specifically incorporated herein by reference.
Ahn, S., and Joyner, A. L. (2005). In vivo analysis of quiescent adult neural stem cells responding to Sonic hedgehog. Nature 437, 894-897.
Arnold, K., Sarkar, A., Yram, M. A., Polo, J. M., Bronson, R., Sengupta, S., Seandel, M., Geijsen, N., and Hochedlinger, K. (2011). Sox2(+) adult stem and progenitor cells are important for tissue regeneration and survival of mice. Cell Stem Cell 9, 317-329.
Balordi, F., and Fishell, G. (2007). Mosaic removal of hedgehog signaling in the adult SVZ reveals that the residual wild-type stem cells have a limited capacity for self-renewal. J Neuroscience 27, 14248-14259.
Beckervordersandforth, R., Tripathi, P., Ninkovic, J., Bayam, E., Lepier, A., Stempfhuber, B., Kirchhoff, F., Hirrlinger, J., Haslinger, A., Lie, D. C., et al. (2010). In vivo fate mapping and expression analysis reveals molecular hallmarks of prospectively isolated adult neural stem cells. Cell Stem Cell 7, 744-758.
Birbrair, A., Wang, Z.-M., Messi, M. L., Enikolopov, G. N., and Delbono, O. (2011). Nestin-GFP transgene reveals neural precursor cells in adult skeletal muscle. PLoS One 6, e16816.
Bruggeman, S. W. M., Hulsman, D., Tanger, E., Buckle, T., Blom, M., Zevenhoven, J., van Tellingen, O., and van Lohuizen, M. (2007). Bmil controls tumor development in an Ink4a/Arf-independent manner in a mouse model for glioma. Cancer Cell 12, 328-341.
Bruggeman, S. W. M., Valk-Lingbeek, M. E., van der Stoop, P. P. M., Jacobs, J. J. L., Kieboom, K., Tanger, E., Hulsman, D., Leung, C., Arsenijevic, Y., Marino, S., et al. (2005). Ink4a and Arf differentially affect cell proliferation and neural stem cell self-renewal in Bmi1-deficient mice. Genes Dev 19, 1438-1443.
Capela, A., and Temple, S. (2002). LeX/ssea-1 is expressed by adult mouse CNS stem cells, identifying them as nonependymal. Neuron 35, 865-875.
Chen, J., Kwon, C.-H., Lin, L., Li, Y., and Parada, L. F. (2009). Inducible site-specific recombination in neural stem/progenitor cells. Genesis 47, 122-131.
Chuikov, S., Levi, B. P., Smith, M. L., and Morrison, S. J. (2010). Prdm16 promotes stem cell maintenance in multiple tissues, partly by regulating oxidative stress. Nature Cell Biology 12, 999-1006.
Davis, A. A., and Temple, S. (1994). A self-renewing multipotential stem cell in embryonic rat cerebral cortex. Nature 372, 263-266.
Ding, L., Saunders, T. L., Enikolopov, G., and Morrison, S. J. (2012). Endothelial and perivascular cells maintain haematopoietic stem cells. Nature 481, 457-462.
Doetsch, F., Caille, I., Lim, D. A., Garcia-Verdugo, J. M., and Alvarez-Buylla, A. (1999a). Subventricular zone astrocytes are neural stem cells in the adult mammalian brain. Cell 97, 703-716.
Doetsch, F., Garcia-Verdugo, J. M., and Alvarez-Buylla, A. (1997). Cellular composition and three-dimensional organization of the subventricular germinal zone in the adult mammalian brain. J Neuroscience 17, 5046-5061.
Doetsch, F., Garcia-Verdugo, J. M., and Alvarez-Buylla, A. (1999b). Regeneration of a germinal layer in the adult mammalian brain. Proceedings of the National Academy of Sciences of the United States of America 96, 11619-11624.
Doetsch, F., Petreanu, L., Caille, I., Garcia-Verdugo, J. M., and Alvarez-Buylla, A. (2002). EGF converts transit-amplifying neurogenic precursors in the adult brain into multipotent stem cells. Neuron 36, 1021-1034.
Fasano, C. A., Phoenix, T. N., Kokovay, E., Lowry, N., Elkabetz, Y., Dimos, J. T., Lemischka, I. R., Studer, L., and Temple, S. (2009). Bmi-1 cooperates with Foxg1 to maintain neural stem cell self-renewal in the forebrain. Genes Dev 23, 561-574.
Garcia, A. D. R., Doan, N. B., Imura, T., Bush, T. G., and Sofroniew, M. V. (2004). GFAP-expressing progenitors are the principal source of constitutive neurogenesis in adult mouse forebrain. Nature Neuroscience 7, 1233-1241.
Garthe, A., Behr, J., and Kempermann, G. (2009). Adult-generated hippocampal neurons allow the flexible use of spatially precise learning strategies. PLoS One 4, e5464.
Giachino, C., Basak, O., Lugert, S., Knuckles, P., Obernier, K., Fiorelli, R., Frank, S., Raineteau, O., Alvarez-Buylla, A., and Taylor, V. (2013). Molecular diversity subdivides the adult forebrain neural stem cell population. Stem Cells 32, 70-84.
Giachino, C., and Taylor, V. (2009). Lineage analysis of quiescent regenerative stem cells in the adult brain by genetic labelling reveals spatially restricted neurogenic niches in the olfactory bulb. Eur J Neurosci 30, 9-24.
He, S., Iwashita, T., Buchstaller, J., Molofsky, A. V., Thomas, D., and Morrison, S. J. (2009). Bmi-1 over-expression in neural stem/progenitor cells increases proliferation and neurogenesis in culture but has little effect on these functions in vivo. Developmental Biology 328, 257-272.
Hirrlinger, P. G., Scheller, A., Braun, C., Hirrlinger, J., and Kirchhoff, F. (2006). Temporal control of gene recombination in astrocytes by transgenic expression of the tamoxifen-inducible DNA recombinase variant CreERT2. Glia 54, 11-20.
Hsieh, J., Aimone, J. B., Kaspar, B. K., Kuwabara, T., Nakashima, K., and Gage, F. H. (2004). IGF-I instructs multipotent adult neural progenitor cells to become oligodendrocytes. J Cell Biol 164, 111-122.
Ihrie, R. A., and Alvarez-Buylla, A. (2011). Lake-front property: a unique germinal niche by the lateral ventricles of the adult brain. Neuron 70, 674-686.
Ihrie, R. A., Shah, J. K., Harwell, C. C., Levine, J. H., Guinto, C. D., Lezameta, M., Kriegstein, A. R., and Alvarez-Buylla, A. (2011). Persistent sonic hedgehog signaling in adult brain determines neural stem cell positional identity. Neuron 71, 250-262.
Imayoshi, I., Sakamoto, M., Ohtsuka, T., Takao, K., Miyakawa, T., Yamaguchi, M., Mori, K., Ikeda, T., Itohara, S., and Kageyama, R. (2008). Roles of continuous neurogenesis in the structural and functional integrity of the adult forebrain. Nature Neuroscience 11,1153-1161.
Imura, T., Kornblum, H. I., and Sofroniew, M. V. (2003). The predominant neural stem cell isolated from postnatal and adult forebrain but not early embryonic forebrain expresses GFAP. J Neuroscience 23, 2824-2832.
Jacobs, J. J., Kieboom, K., Marino, S., DePinho, R. A., and van Lohuizen, M. (1999). The oncogene and Polycomb-group gene bmi-1 regulates cell proliferation and senescence through the ink4a locus. Nature 397, 164-168.
Joseph, N. M., and Morrison, S. J. (2005). Toward an understanding of the physiological function of Mammalian stem cells. Developmental Cell 9, 173-183.
Kim, E. J., Ables, J. L., Dickel, L. K., Eisch, A. J., and Johnson, J. E. (2011). Ascl1 (Mash1) defines cells with long-term neurogenic potential in subgranular and subventricular zones in adult mouse brain. PLoS One 6, e 1 8472.
Kuo, C. T., Mirzadeh, Z., Soriano-Navarro, M., Rasin, M., Wang, D., Shen, J., Sestan, N., Garcia-Verdugo, J., Alvarez-Buylla, A., Jan, L. Y., et al. (2006). Postnatal deletion of Numb/Numblike reveals repair and remodeling capacity in the subventricular neurogenic niche. Cell 127, 1253-1264.
Lagace, D. C., Whitman, M. C., Noonan, M. A., Ables, J. L., DeCarolis, N. A., Arguello, A. A., Donovan, M. H., Fischer, S. J., Farnbauch, L. A., Beech, R. D., et al. (2007). Dynamic contribution of nestin-expressing stem cells to adult neurogenesis. J Neuroscience 27, 12623-12629.
Le Belle, J. E., Orozco, N. M., Paucar, A. A., Saxe, J. P., Mottahedeh, J., Pyle, A. D., Wu, H., and Kornblum, H. I. (2011). Proliferative neural stem cells have high endogenous ROS levels that regulate self-renewal and neurogenesis in a PI3K/Akt-dependant manner. Cell Stem Cell 8, 59-71.
Lee, C., Hu, J., Ralls, S., Kitamura, T., Loh, Y. P., Yang, Y., Mukouyama, Y.-s., and Ahn, S. (2012). The molecular profiles of neural stem cell niche in the adult subventricular zone. PLoS One 7, e50501.
Lessard, J., and Sauvageau, G. (2003). Bmi-1 determines the proliferative capacity of normal and leukaemic stem cells. Nature 423, 255-260.
Liu, P., Jenkins, N. A., and Copeland, N. G. (2003). A highly efficient recombineering-based method for generating conditional knockout mutations. Genome research 13, 476-484.
Lois, C., and Alvarez-Buylla, A. (1994). Long-distance neuronal migration in the adult mammalian brain. Science 264, 1145-1148.
Lois, C., Garcia-Verdugo, J. M., and Alvarez-Buylla, A. (1996). Chain migration of neuronal precursors. Science 271, 978-981.
López-Juárez, A., Howard, J., Ullom, K., Howard, L., Grande, A., Pardo, A., Waclaw, R., Sun, Y.-Y., Yang, D., Kuan, C.-Y., et al. (2013). Gsx2 controls region-specific activation of neural stem cells and injury-induced neurogenesis in the adult subventricular zone. Genes Dev 27, 1272-1287.
Madisen, L., Zwingman, T. A., Sunkin, S. M., Oh, S. W., Zariwala, H. A., Gu, H., Ng, L. L., Palmiter, R. D., Hawrylycz, M. J., Jones, A. R., et al. (2010). A robust and high-throughput Cre reporting and characterization system for the whole mouse brain. Nature Neuroscience 13, 133-140.
Maslov, A. Y., Barone, T. A., Plunkett, R. J., and Pruitt, S. C. (2004). Neural stem cell detection, characterization, and age-related changes in the subventricular zone of mice. J Neuroscience 24, 1726-1733.
Menn, B., Garcia-Verdugo, J. M., Yaschine, C., Gonzalez-Perez, O., Rowitch, D., and Alvarez-Buylla, A. (2006). Origin of oligodendrocytes in the subventricular zone of the adult brain. J Neuroscience 26, 7907-7918.
Merkle, F. T., Fuentealba, L. C., Sanders, T. A., Magno, L., Kessaris, N., and Alvarez-Buylla, A. (2014). Adult neural stem cells in distinct microdomains generate previously unknown interneuron types. Nature Neuroscience 17, 207-214.
Merkle, F. T., Mirzadeh, Z., and Alvarez-Buylla, A. (2007). Mosaic organization of neural stem cells in the adult brain. Science 317, 381-384.
Mirzadeh, Z., Doetsch, F., Sawamoto, K., Wichterle, H., and Alvarez-Buylla, A. (2010). The subventricular zone en-face: wholemount staining and ependymal flow. J Vis Exp (39), e1938.
Mirzadeh, Z., Merkle, F. T., Soriano-Navarro, M., Garcia-Verdugo, J. M., and Alvarez-Buylla, A. (2008). Neural stem cells confer unique pinwheel architecture to the ventricular surface in neurogenic regions of the adult brain. Cell Stem Cell 3, 265-278.
Molofsky, A. V., He, S., Bydon, M., Morrison, S. J., and Pardal, R. (2005). Bmi-1 promotes neural stem cell self-renewal and neural development but not mouse growth and survival by repressing the p16Ink4a and p19Arf senescence pathways. Genes Dev 19, 1432-1437.
Molofsky, A. V., Pardal, R., Iwashita, T., Park, I.-K., Clarke, M. F., and Morrison, S. J. (2003). Bmi-1 dependence distinguishes neural stem cell self-renewal from progenitor proliferation. Nature 425, 962-967.
Molofsky, A. V., Slutsky, S. G., Joseph, N. M., He, S., Pardal, R., Krishnamurthy, J., Sharpless, N. E., and Morrison, S. J. (2006). Increasing p16INK4a expression decreases forebrain progenitors and neurogenesis during ageing. Nature 443, 448-452.
Morshead, C. M., Garcia, A. D., Sofroniew, M. V., and van Der Kooy, D. (2003). The ablation of glial fibrillary acidic protein-positive cells from the adult central nervous system results in the loss of forebrain neural stem cells but not retinal stem cells. Eur J Neurosci 18, 76-84.
Morshead, C. M., Reynolds, B. A., Craig, C. G., McBurney, M. W., Staines, W. A., Morassutti, D., Weiss, S., and van der Kooy, D. (1994). Neural stem cells in the adult mammalian forebrain: a relatively quiescent subpopulation of subependymal cells. Neuron 13, 1071-1082.
Nait-Oumesmar, B., Decker, L., Lachapelle, F., Avellana-Adalid, V., Bachelin, C., and Baron-Van Evercooren, A. (1999). Progenitor cells of the adult mouse subventricular zone proliferate, migrate and differentiate into oligodendrocytes after demyelination. Eur J Neurosci 11, 4357-4366.
Nishino, J., Kim, I., Chada, K., and Morrison, S. J. (2008). Hmga2 promotes neural stem cell self-renewal in young but not old mice by reducing p16Ink4a and p19Arf Expression. Cell 135 , 227-239.
Oguro, H., Iwama, A., Morita, Y., Kamijo, T., van Lohuizen, M., and Nakauchi, H. (2006). Differential impact of Ink4a and Arf on hematopoietic stem cells and their bone marrow microenvironment in Bmil-deficient mice. J Exp Med 203, 2247-2253.
Oguro, H., Yuan, J., Ichikawa, H., Ikawa, T., Yamazaki, S., Kawamoto, H., Nakauchi, H., and Iwama, A. (2010). Poised lineage specification in multipotential hematopoietic stem and progenitor cells by the polycomb protein Bmil. Cell Stem Cell 6, 279-286.
Park, I. K., Qian, D., Kiel, M., Becker, M. W., Pihalja, M., Weissman, I. L., Morrison, S. J., and Clarke, M. F. (2003). Bmi-1 is required for maintenance of adult self-renewing haematopoietic stem cells. Nature 423, 302-305.
Pastrana, E., Cheng, L.-C., and Doetsch, F. (2009). Simultaneous prospective purification of adult subventricular zone neural stem cells and their progeny. Proceedings of the National Academy of Sciences of the United States of America 106, 6387-6392.
Pastrana, E., Silva-Vargas, V., and Doetsch, F. (2011). Eyes wide open: a critical review of sphere-formation as an assay for stem cells. Cell Stem Cell 8, 486-498.
Reynolds, B. A., and Rietze, R. L. (2005). Neural stem cells and neurospheres--re-evaluating the relationship. Nat Methods 2, 333-336.
Reynolds, B. A., and Weiss, S. (1992). Generation of neurons and astrocytes from isolated cells of the adult mammalian central nervous system. Science 255, 1707-1710.
Rodriguez, C. I., Buchholz, F., Galloway, J., Sequerra, R., Kasper, J., Ayala, R., Stewart, A. F., and Dymecki, S. M. (2000). High-efficiency deleter mice show that FLPe is an alternative to Cre-loxP. Nature genetics 25, 139-140.
Schaefer, B. C., Schaefer, M. L., Kappler, J. W., Marrack, P., and Kedl, R. M. (2001). Observation of antigen-dependent CD8+ T-cell/ dendritic cell interactions in vivo. Cell Immunol 214, 110-122.
Singec, I., Knoth, R., Meyer, R. P., Maciaczyk, J., Volk, B., Nikkhah, G., Frotscher, M., and Snyder, E. Y. (2006). Defining the actual sensitivity and specificity of the neurosphere assay in stem cell biology. Nat Methods 3, 801-806.
Taniguchi, H., He, M., Wu, P., Kim, S., Paik, R., Sugino, K., Kvitsiani, D., Kvitsani, D., Fu, Y., Lu, J., et al. (2011). A resource of Cre driver lines for genetic targeting of GABAergic neurons in cerebral cortex. Neuron 71, 995-1013.
Tronche, F., Kellendonk, C., Kretz, O., Gass, P., Anlag, K., Orban, P. C., Bock, R., Klein, R., and Schutz, G. (1999). Disruption of the glucocorticoid receptor gene in the nervous system results in reduced anxiety. Nature genetics 23, 99-103.
van der Lugt, N. M., Domen, J., Linders, K., van Roon, M., Robanus-Maandag, E., to Riele, H., van der Valk, M., Deschamps, J., Sofroniew, M., van Lohuizen, M., et al. (1994). Posterior transformation, neurological abnormalities, and severe hematopoietic defects in mice with a targeted deletion of the bmi-1 proto-oncogene. Genes Dev 8, 757-769.
Wang, Y., Rattner, A., Zhou, Y., Williams, J., Smallwood, P. M., and Nathans, J. (2012). Norrin/Frizzled4 signaling in retinal vascular development and blood brain barrier plasticity. Cell 151, 1332-1344.
Zencak, D., Lingbeek, M., Kostic, C., Tekaya, M., Tanger, E., Hornfeld, D., Jaquet, M., Munier, F. L., Schorderet, D. F., van Lohuizen, M., et al. (2005). Bmil loss produces an increase in astroglial cells and a decrease in neural stem cell population and proliferation. J Neuroscience 25, 5774-5783.

1. An isolated neurosphere-initiating cell population characterized by expression of:
moderate levels of Glast (˜10² to ˜6×10³ arbitrary expression units as on FIG. 1B), high levels of EGFR (at least ˜10³ arbitrary expression units as on FIG. 1B), high levels of PlexinB2 (at least ˜10³ arbitrary expression units as on FIG. 1B), negative to low levels of CD24 (less than ˜5×10² arbitrary expression units as on FIG. 1B), negative to low levels of O4 and PSA-NCAM (less than ˜10³ arbitrary expression units as on FIG. 1B), and negative for the hematopoietic markers Ter119 and CD45 (less than ˜10² arbitrary expression units as on FIG. 1B).

2. The isolated cell population of claim 1, wherein cells are subventricular zone (SVZ) cells.

3. The isolated cell population of claim 1, at least 20% of which form neurospheres upon addition to non-adherent cultures.

4. The isolated cell population of claim 1, at least 20% of which form neurospheres that undergo multilineage differentiation in culture.

5. The isolated cell population of claim 1, which divides frequently in the subventricular zone (SVZ) and is short-lived in vivo.

6. The isolated cell population of claim 1, which is a cell population sensitive to temozolomide (TMZ) treatment. (Original) The isolated cell population of claim 1, which requires Bmi-1 for its maintenance in vivo.

8. An isolated neural stem cell population characterized by expression of:
high levels of Glast (at least ˜10⁴ arbitrary expression units as on FIG. 3E), negative to low levels of Epidermal Growth Factor Receptor (EGFR) (less than ˜5×10² arbitrary expression units as on FIG. 3E), moderate levels of PlexinB2 (less than ˜10³ arbitrary expression units as on FIG. 3E), negative to low levels of CD24 (less than ˜10² arbitrary expression units as on FIG. 3E), negative to low levels of O4 and PSA-NCAM (less than ˜10³ arbitrary expression units as on FIG. 3E), and negative for the hematopoietic markers Ter119 and CD45 (less than ˜10² arbitrary expression units as on FIG. 1B); and
resistance to temozolomide (TMZ).

9. The isolated cell population of claim 8, wherein cells are subventricular zone (SVZ) cells.

10. The isolated cell population of claim 8, enriched for GFAP⁺ cells relative to unfractionated subventricular zone (SVZ) cells.

11. The isolated cell population of claim 8, enriched for cells recombined by GFAP-CreERT² relative to unfractionated subventricular zone (SVZ) cells.

12. The isolated cell population of claim 8, enriched with cells fated to give rise to neurosphere-initiating cells in the subventricular zone (SVZ).

13. The isolated cell population of claim 8, which is highly quiescent.

14. The isolated cell population of claim 8, which requires Bmi-1 for maintenance in vivo.

15. A method of isolating a neurosphere initiating cell population comprising:
(a) providing a cell population from adult mouse brain; and
(b) selecting cells exhibiting expression of moderate levels of Glast, high levels of Epidermal Growth Factor Receptor (EGFR), high levels of PlexinB2, negative to low levels of CD24, negative to low levels of O4 and PSA-NCAM, and negative for the hematopoietic markers Ter119 and CD45.

16. The method of claim 15, further comprising culturing cells selected in step (b).

17. The method of claim 15, further comprising treating the cells selected in step (b) with temozolomide (TMZ).

18. (canceled)

19. The method of claim 15, further comprising removing debris from enzymatically dissociated SVZ cells prior to step (a).

20. The method of claim 15, wherein EDTA is added to the cell population of step (a).

21. The method of claim 16, further comprising maintaining the pH of the culture medium at a neutral range.

22. (canceled)

23. The method of claim 16, further comprising adding a Rock inhibitor and/or IGF1 to the culture medium.

24. The method of claim 15, further comprising preventing aggregation following selection.

25. A method of isolating a neural stem cell population comprising:
(a) providing an enzymatically dissociated cell population from adult mouse brain; and
(b) selecting cells that exhibit high levels of Glast, negative to low levels of Epidermal Growth Factor Receptor (EGFR), moderate levels of PlexinB2, negative to low levels of CD24, negative to low levels of O4 and PSA-NCAM, and negative for the hematopoietic markers Ter119 and CD45.

26-44. (canceled)


SEQUENCE LISTING (10 sequences)

SEQ ID NO: 1 - DNA, 21 bp, Artificial Sequence
  Synthetic Primer
ccaatggctccaatgaagacc

SEQ ID NO: 2 - DNA, 20 bp, Artificial Sequence
  Synthetic Primer
ttgctgctgggcatcgtaag

SEQ ID NO: 3 - DNA, 19 bp, Artificial Sequence
  Synthetic Primer
cgctctttccgggatcttt

SEQ ID NO: 4 - DNA, 21 bp, Artificial Sequence
  Synthetic Primer
ctccacacaggacacacatta

SEQ ID NO: 5 - DNA, 18 bp, Artificial Sequence
  Synthetic Primer
gtgtgcatgacgtgcggg

SEQ ID NO: 6 - DNA, 22 bp, Artificial Sequence
  Synthetic Primer
gcagttcgaatctgcaccgtag

SEQ ID NO: 7 - DNA, 22 bp, Artificial Sequence
  Synthetic Primer
cgtcgacaacggctccggcatg

SEQ ID NO: 8 - DNA, 23 bp, Artificial Sequence
  Synthetic Primer
gggcctcgtcacccacataggag

SEQ ID NO: 9 - DNA, 20 bp, Artificial Sequence
  Synthetic Primer
gggcccagagctttcccacg

SEQ ID NO: 10 - DNA, 21 bp, Artificial Sequence
  Synthetic Primer
gggcatgcaccagaccctgtg